(19) United States
(12) Patent Application Publication
(10) Pub. No.: US 2009/0075916 A1
(43) Pub. Date: Mar. 19, 2009

(54) Use of Macrolide Derivatives for Treating Acne

(76) Inventors: Dilip J. Upadhyay, Thane (IN); Anjan Chakrabarti, Gurgaon (IN); Biswajit Das, Gurgaon (IN); Radhakrishnan Gowrishankar, Gurgaon (IN)

Correspondence Address: RANBAXY INC., 600 COLLEGE ROAD EAST, SUITE 2100, PRINCETON, NJ, 08540, US

(21) Appl. No.: 12/094,645

(22) PCT Filed: Nov. 22, 2006
(86) PCT No.: PCT/IB06/54391

§ 371 Date: May 22, 2008

(30) Foreign Application Priority Data
Nov. 23, 2005 (IN) 3143/DEL/2005

(51) Int. Cl. A61K 31/7048; A61P 17/10

(52) U.S. Cl. 514/29

(57) ABSTRACT

This invention relates to 14-membered macrolide derivatives for treating or preventing acne vulgaris and associated inflammatory conditions. Also provided herein are medicaments or pharmaceutical compositions comprising one or more 14-membered macrolide derivatives together with one or more pharmaceutically acceptable carriers, excipients, diluents or mixture thereof, and methods for treating or preventing acne vulgaris and associated inflammatory conditions.

### FIELD OF THE INVENTION

This invention relates to 14-membered macrolides derivatives for treating or preventing acne vulgaris and inflammatory conditions thereof. Also provided herein are pharmaceutical compositions comprising one or more 14-membered macrolides derivatives together with one or more pharmaceutically acceptable carriers, excipients, diluents or mixture thereof, and methods for treating or preventing acne vulgaris and inflammatory condition thereof.

### BACKGROUND OF THE INVENTION

Acne is an inflammation of the skin that affects people from every country and every culture around the world. It is a disease of the pilosebaceous units (PSUs), which consist of a sebaceous gland connected to a canal, called a follicle that contains a fine hair. The sebaceous glands make an oily substance called sebum that normally empties onto the skin surface through the opening of the follicle, commonly called a pore. Cells called keratinocytes line the follicle. The hair, sebum, and keratinocytes that fill the narrow follicle may produce a plug, which is an early sign of acne. The plug prevents sebum from reaching the surface of the skin through a pore. The mixture of oil and cells allows the most common anaerobes, Propionibacterium acnes (P. acnes) that normally live on the skin, to grow in the plugged follicle. These bacteria produce chemicals and enzymes and attract white blood cells that cause inflammation (e.g., swelling, redness, heat or pain). Propionibacterium acnes associated with acne vulgaris and linked to certain cases of endocarditis, anaerobic arthritis, wound infections and abscesses.

Acne Vulgaris is the most common form of acne, which includes several types of lesions. For example, mild to moderate acne vulgaris (e.g., whitehead, blackhead, papules or pimples) and severe acne vulgaris (e.g., nodules or cysts).

The exact cause of acne is unknown, but it is believed that factors like increase in hormones called androgens (male sex hormones) and heredity or genetics may be responsible. Drug treatment is aimed at reducing several problems that play a part in causing acne, for example, abnormal clumping of cells in the follicles, increased oil production, bacteria, and inflammation.

Therapies for treating acne and inflammatory conditions thereof have been described in the prior art as set forth below.

U.S. Pat. No. 5,910,312 discloses acne treatment composition comprising effective amount of benzoyl peroxide (or salicylic acid), a vasoconstrictor and an inert carrier. U.S. Pat. No. 5,962,517 discloses pharmaceutical composition comprising at least one of a vitamin A source, a carotenoid component, a vitamin B₆ source, and a zinc component, in an amount sufficient to reduce the redness and blemishes associated with acne. U.S. Pat. No. 6,262,117 discloses method and composition for treating acne. It provides a method for treating acne vulgaris by serially applying a topical composition of azelaic acid and a topical composition of benzoyl peroxide. U.S. Pat. No. 3,969,516 discloses topical clindamycin to treat acne vulgaris. U.S. Pat. Nos. 4,323,558; 4,505,896; 4,607,101 and 4,018,918 disclose combinations of clindamycin and other compounds active for the treatment of acne vulgaris. U.S. Pat. No. 4,387,107 discloses benzoyl peroxide for treating acne vulgaris. U.S. Pat. No. 4,386,104 discloses azelaic acid for treating acne topically or systemically. Other agents used for treating acne vulgaris include erythromycin, tetracycline, isotretinoin or herbal preparations.

Despite the existence of such drugs for treating or preventing acne vulgaris and inflammatory conditions thereof, there remains a need for safe and effective drugs for treating or preventing acne vulgaris and inflammatory conditions thereof.

### SUMMARY OF THE INVENTION

Accordingly, provided herein the use of 14-membered macrolide derivatives for treating or preventing acne vulgaris and inflammatory conditions thereof, wherein 14-membered macrolide derivatives are selected from:

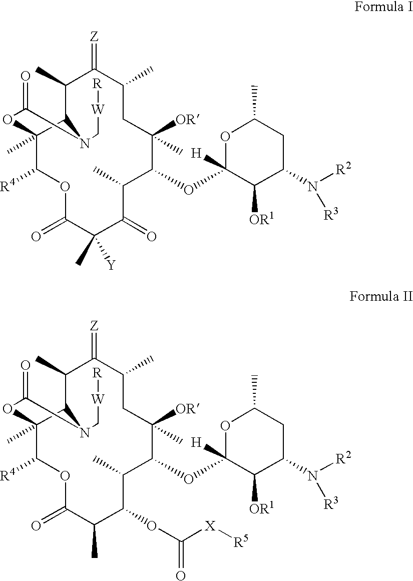

Pharmaceutically acceptable salts, pharmaceutically acceptable solvates, enantiomers, diastereomers or polymorphs thereof, wherein:

R¹ can be hydrogen or a hydroxyl-protecting group;

R² and R³ can independently be hydrogen, alkyl, alkenyl, alkynyl, cycloalkyl, aryl, heterocycle, aralkyl, (heterocycle)alkyl or COR¹¹, wherein R¹¹ can be hydrogen, alkyl or aralkyl, with the proviso that R² and R³ can be simultaneously methyl only when Y is hydrogen;

W can be alkenyl, -G(CH₂)_{q}J-, —CR⁹R¹⁰, —NR⁹— or —SO₂, wherein

q can be an integer of from 2 to 6;

G can be no atom, —CO, —CS or —SO₂;

R⁹ and R¹⁰ can independently be hydrogen or alkyl; and

J can be no atom, —CR⁹R¹⁰ or N(R¹²)(CH₂)ₘ, wherein m can be an integer of from 0 to 6; R⁹ and R¹⁰ can be the same as defined earlier; and R¹² can be hydrogen, alkyl, alkylene, alkynyl, COR⁸ or —(CH₂)ₘ—R⁸, wherein R⁸ can be alkyl, aryl or heterocycle;

R can be no atom, hydrogen, aryl or heterocycle;

R⁴ can be alkyl, alkenyl or alkynyl;

R⁵ can be alkyl, aryl or heterocycle;

R′ can be alkyl or —(CH₂)ᵣ—U, wherein r can be an integer of from 1 to 4 and U can be alkenyl or alkynyl;
X can be -Q(CH₂)ₖ—, {wherein k can be an integer of from 1 to 6, Q can be no atom, —NR⁹— or oxygen [wherein R⁹ can be hydrogen or alkyl]}, further alkylene chain of -Q(CH₂)ₖ— can be optionally substituted with alkyl, hydroxy or alkoxy;

Y can be hydrogen, halogen, cyano or alkyl; and

Z can be oxygen, sulfur or NOR¹¹, wherein R¹¹ can be the same as defined earlier.

Also provided herein are pharmaceutical compositions comprising therapeutically effective amounts of one or more 14-membered macrolide derivatives of Formula I, II or mixture thereof, together with one or more pharmaceutically acceptable carriers, excipients, diluents or mixture thereof.

Also provided herein are methods for treating or preventing acne vulgaris and inflammatory conditions thereof comprising administering to a mammal in need thereof therapeutically effective amounts of one or more 14-membered macrolide derivatives of Formula I, II or mixture thereof.

Also provided herein are methods for treating or preventing acne vulgaris and inflammatory conditions thereof comprising administering to a mammal in need thereof pharmaceutical compositions comprising therapeutically effective amounts of one or more 14-membered macrolide derivatives of Formula I, II or mixture thereof, together with one or more pharmaceutically acceptable carriers, excipients, diluents or mixture thereof.

Also provided herein are methods for treating or preventing acne vulgaris and inflammatory conditions thereof comprising administering to a mammal in need thereof therapeutically effective amounts of one or more 14-membered macrolide derivatives of Formula I, II or mixture thereof, in combination with one or more therapeutic agents selected from alcohol, benzoyl peroxide, clindamycin, tretinoin, vitamin E, vitamin A and its derivatives, tetracycline, isotretinoin, vitamin C, vitamin D, chaparral, dandelion root, licoric root, Echinacea, kelp, cayenine, sassafras, elder flowers, pantothenic acid, para amino benzoic acid, biotin, cholin, inositol, folic acid, calcium, magnesium, potassium, vitamin B₆, zinc, carotenoid, azelaic acid, and other therapeutic agents, which can be used to treat acne or condition the skin.

Also provided herein are methods for treating or preventing acne vulgaris and inflammatory conditions thereof comprising administering to a mammal in need thereof therapeutically effective amount of a combination pharmaceutical composition.

Other objects will be set forth in accompanying detailed description, which follows and in the part will be apparent from the description or may be learnt by the practice of the invention. However, It should be understood that the following detailed description are given by way of illustration only since various changes and modifications within the spirit and scope of the invention will become apparent to those skilled in the art and are intended to be encompassed within the scope of this invention.

### DETAILED DESCRIPTION OF THE INVENTION

Generally, provided herein are 14-membered macrolide derivatives for treating or preventing acne vulgaris and inflammatory conditions thereof.

Thus in one aspect, provided herein are 14 membered macrolide derivatives for treating or preventing acne vulgaris and inflammatory conditions thereof, wherein 14-membered macrolide derivatives are selected from:

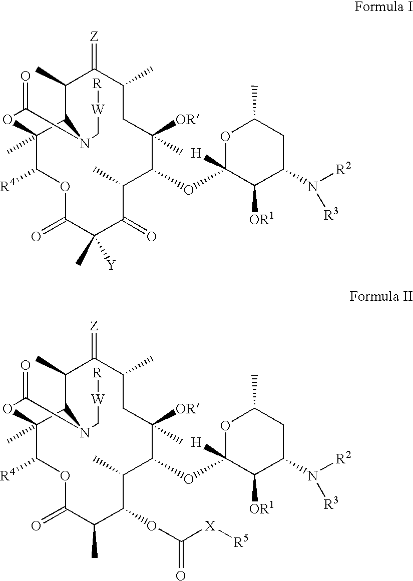

Pharmaceutically acceptable salts, pharmaceutically acceptable solvates, enantiomers, diastereomers or polymorphs thereof, wherein:
R¹ can be hydrogen or a hydroxyl-protecting group;
R² and R³ can independently be hydrogen, alkyl, alkenyl, alkynyl, cycloalkyl, aryl, heterocycle, aralkyl, (heterocycle)alkyl or COR¹¹, wherein R¹¹ can be hydrogen, alkyl or aralkyl, with the proviso that R² and R³ can be simultaneously methyl only when Y is hydrogen;
W can be alkenyl, -G(CH₂)_{q}J-, —CR⁹R¹⁰, —NR⁹— or —SO₂, wherein
   q can be an integer of from 2 to 6;
   G can be no atom, —CO, —CS or —SO₂;
   R⁹ and R¹⁰ can independently be hydrogen or alkyl; and
   J can be no atom, —CR⁹R¹⁰ or N(R¹²)(CH₂)ₘ, wherein m can be an integer of from 0 to 6; R⁹ and R¹⁰ can be the same as defined earlier; and R¹² can be hydrogen, alkyl, alkylene, alkynyl, COR⁸ or —(CH₂)ₘ—R⁸, wherein R⁸ can be alkyl, aryl or heterocycle;
R can be no atom, hydrogen, aryl or heterocycle;
R⁴ can be alkyl, alkenyl or alkynyl;
R⁵ can be alkyl, aryl or heterocycle;
R′ can be alkyl or —(CH₂)ᵣ—U, wherein r can be an integer of from 1 to 4 and U can be alkenyl or alkynyl;
X can be -Q(CH₂)ₖ—, {wherein k can be an integer of from 1 to 6, Q can be no atom, —NR⁹— or oxygen [wherein R⁹ can be hydrogen or alkyl]}, further alkylene chain of -Q(CH₂)ₖ— can be optionally substituted with alkyl, hydroxy or alkoxy;
Y can be hydrogen, halogen, cyano or alkyl; and
Z can be oxygen, sulfur or NOR¹¹, wherein R¹¹ can be the same as defined earlier.

Compounds of Formula I have been described in our PCT application Nos. WO 2006/080954, WO 2006/046112 and in our copending Indian patent application 1383/DEL/2005 and 3138/DEL/2005. Compounds of Formula II have been described in our PCT application Nos. WO 2006/013409, WO 2005/030786 and WO 2006/035301.

In another aspect, provided herein are specific compounds of Formulae I and II for treating acne vulgaris and inflammatory conditions thereof. These are:

| Comp. | |
|---|---|
| No. | Nomenclature |
| 1. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((3-(imidazol-1-yl)-propyl)- |
| | imino)]erythromycin A |
| 2. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(1H-imidazo[4,5-b]- |
| | pyridin-1-yl)-butyl)-imino)]erythromycin A |
| 3. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(3H-imidazo[4,5-b]- |
| | pyridin-1-yl)-butyl)-imino)]erythromycin A |
| 4. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(benzoimidazol-1-yl)- |
| | butyl)-imino)]erythromycin A |
| 5. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(imidazol-1-yl)-butyl)- |
| | imino)]erythromycin A |
| 6. | 2-α-Fluoro-5-O-(3′-N-desmethyl-3′-N-cyclopropyl)-desosaminyl-11,12-dideoxy- |
| | 3-O-decladinosyl-6-O-methyl-3-oxo-12,11-[oxycarbonyl-((3-(imidazol-1-yl)- |
| | propyl)-imino)]erythromycin A, |
| 7. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((3-(imidazol-1-yl)- |
| | propyl)-imino)]erythromycin A |
| 8. | 2-α-Fluoro-5-O-(3′-N-desmethyl-3′-N-propyl)-desosaminyl-11,12-dideoxy-3-O- |
| | decladinosyl-6-O-methyl-3-oxo-12,11-[oxycarbonyl-((3-(imidazol-1-yl)-propyl)- |
| | imino)]erythromycin A |
| 9. | 2-α-Fluoro-5-O-(3′-N-desmethyl-3′-N-propyl)-desosaminyl-11,12-dideoxy-3-O- |
| | decladinosyl-6-O-methyl-3-oxo-12,11-[oxycarbonyl-((4-(benzoimidazol-1-yl)- |
| | butyl)-imino)]erythromycin A |
| 10. | 2-α-Fluoro-5-O-(3′-N-desmethyl-3′-N-cyclopropyl)-desosaminyl-11,12-dideoxy- |
| | 3-O-decladinosyl-6-O-methyl-3-oxo-12,11-[oxycarbonyl-((4-(pyrrolo-[2,3- |
| | b]pyridin-1-yl)-butyl)-imino)]erythromycin A |
| 11. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(pyrrolo-[2,3- |
| | b]pyridin-1-yl)-butyl)-imino)]erythromycin A |
| 12. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(benzoimidazol-1-yl)- |
| | butyl)-imino)]erythromycin A |
| 13. | 2-α-Fluoro-5-O-(3′-N-desmethyl-3′-N-cyclopropyl)-desosaminyl-11,12-dideoxy- |
| | 3-O-decladinosyl-6-O-methyl-3-oxo-12,11-[oxycarbonyl-((4-(benzoimidazol-1- |
| | yl)-butyl)-imino)]erythromycin A |
| 14. | 2-α-Fluoro-5-O-(3′-N-desmethyl-3′-N-n-propyl)-desosaminyl-11,12-dideoxy-3- |
| | O-decladinosyl-6-O-methyl-3-oxo-12,11-[oxycarbonyl-((4-(imidazol-1-yl)- |
| | butyl)-imino)]erythromycin A |
| 15. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(pyrrolo-[2,3-b]pyridin- |
| | 1-yl)-butyl)-imino)]erythromycin A |
| 16. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(1H-imidazo[4,5-b]- |
| | pyridin-1-yl)-butyl)-imino)]erythromycin A |
| 17. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(purin-9-yl)-butyl)- |
| | imino)]erythromycin A |
| 18. | 2-α-Fluoro-5-O-(3′-N-desmethyl-3′-N-cyclopropyl)-desosaminyl-11,12-dideoxy- |
| | 3-O-decladinosyl-6-O-methyl-3-oxo-12,11-[oxycarbonyl-((4-(3H-imidazo[4,5-b]- |
| | pyridin-3-yl)-butyl)-imino)]erythromycin A |
| 19. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyridin-3-yl)- |
| | imidazol-1-yl)-butyl)-imino)]erythromycin A |
| 20. | 2α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(((N′-methyl-N′-quinolin-4- |
| | ylmethyl)-2-aminoethyl)-imino)]erythromycin A |
| 21. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(((N′-methyl-N′-pyridine-3- |
| | ylmethyl)-2-aminoethyl)-imino)]erythromycin A |
| 22. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(((N′-methyl-N-pyridine-4- |
| | ylmethyl)-2-aminoethyl)-imino)]erythromycin A |
| 23. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(((N′-methyl-N′-pyridine-2- |
| | ylmethyl)-2-aminoethyl)-imino)]erythromycin A |
| 24. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-(((N′-methyl-N′-pyridine-2- |
| | ylmethyl)-2-aminoethyl)-imino)]erythromycin A |
| 25. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-(((N′-methyl-N′-pyridine-3- |
| | ylmethyl)-2-aminoethyl)-imino)]erythromycin A |
| 26. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-(((N′-methyl-N′-pyridine-4- |
| | ylmethyl)-2-aminoethyl)-imino)]erythromycin A |
| 27. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-(((N′-methyl-N′-quinoline-4- |
| | ylmethyl)-2-aminoethyl)-imino)]erythromycin A |
| 28. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(((N′-pyridine-3-ylmethyl)- |
| | 2-aminoethyl)-imino)]erythromycin A |
| 29. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-(((N′-acetyl-N′-pyridine-3- |
| | ylmethyl)-2-aminoethyl)-imino)]erythromycin A |
| 30. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-(((N′-pyridine-3-ylmethyl)- |
| | 2-aminoethyl)-imino)]erythromycin A |
| 31. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(((N′-acetyl-N′-pyridine-3- |
| | ylmethyl)-2-aminoethyl)-imino)]erythromycin A |
| 32. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(((N′,N′-di-pyridine-3-yl- |
| | methyl)-2-aminoethyl)-imino)]erythromycin A |
| 33. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-(((N′,N′-di-pyridine-3-yl- |
| | methyl)-2-aminoethyl)-imino)]erythromycin A |
| 34. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(quinolin-3-yl)-butyl)- |
| | imino)]erythromycin A |
| 35. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(quinolin-8-yl)-butyl)- |
| | imino)]erythromycin A |
| 36. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(pyridin-2-yl)-butyl)- |
| | imino)]erythromycin A |
| 37. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(pyridin-3-yl)-butyl)- |
| | imino)]erythromycin A |
| 38. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(pyridin-4-yl)-butyl)- |
| | imino)]erythromycin A |
| 39. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(pyridin-2-yl)-butyl)- |
| | imino)]erythromycin A |
| 40. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(pyridin-4-yl)-butyl)- |
| | imino)]erythromycin A |
| 41. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(pyridin-3-yl)-pentyl)- |
| | imino)]erythromycin A |
| 42. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(pyridin-3-yl)-butyl)- |
| | imino)]erythromycin A |
| 43. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(indol-1-yl)-butyl)- |
| | imino)]erythromycin A |
| 44. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-phenylimidazol-1-yl)- |
| | butyl)-imino)]erythromycin A |
| 45. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5-nitroindol-1-yl)- |
| | butyl)-imino)]erythromycin A |
| 46. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(1H-imidazo[4,5- |
| | c]pyridin-1-yl)-butyl)-imino)]erythromycin A |
| 47. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(benzotriazol-2-yl)- |
| | butyl)-imino)]erythromycin A |
| 48. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(3H-imidazo[4,5- |
| | c]pyridin-3-yl)-butyl)-imino)]erythromycin A |
| 49. | 2-α-Fluoro-5-O-(3′-N-desmethyl-3′-N-cyclopropyl)-desosaminyl-11,12-dideoxy- |
| | 3-O-decladinosyl-6-O-methyl-3-oxo-12,11-[oxycarbonyl-((4-(4-phenylimidazol- |
| | 1-yl)-butyl)-imino)]erythromycin A |
| 50. | 2-α-Fluoro-5-O-(3′-N-desmethyl-3′-N-cyclopropyl)-desosaminyl-11,12-dideoxy- |
| | 3-O-decladinosyl-6-O-methyl-3-oxo-12,11-[oxycarbonyl-((4-(benzotriazol-1-yl)- |
| | butyl)-imino)]erythromycin A |
| 51. | 2-α-Fluoro-5-O-(3′-N-desmethyl-3′-N-cyclopropyl)-desosaminyl-11,12-dideoxy- |
| | 3-O-decladinosyl-6-O-methyl-3-oxo-12,11-[oxycarbonyl-((4-(benzotriazol-2-yl)- |
| | butyl)-imino)]erythromycin A |
| 52. | 2-α-Fluoro-5-O-(3′-N-desmethyl-3′-N-cyclopropyl)-desosaminyl-11,12-dideoxy- |
| | 3-O-decladinosyl-6-O-methyl-3-oxo-12,11-[oxycarbonyl-((4-(1H)-imdazo[4,5- |
| | b]pyridin-1-yl)-butyl)-imino)]erythromycin A |
| 53. | 2-α-Fluoro-5-O-(3′-N-desmethyl-3′-N-cyclopropyl)-desosaminyl-11,12-dideoxy- |
| | 3-O-decladinosyl-6-O-methyl-3-oxo-12,11-[oxycarbonyl-((4-(indol-1-yl)-butyl)- |
| | imino)]erythromycin A |
| 54. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(benzotriazol-1-yl)- |
| | butyl)-imino)]erythromycin A |
| 55. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(benzoimidazol-1-yl)- |
| | butyl)-imino)]erythromycin A |
| 56. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(3H-imidazo[4,5- |
| | b]pyridin-3-yl)-butyl)-imino)]erythromycin A |
| 57. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(4-phenylimidazol-1- |
| | yl)-butyl)-imino)]erythromycin A |
| 58. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(benzotriazol-2-yl)- |
| | butyl)-imino)]erythromycin A |
| 59. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(indol-1-yl)-butyl)- |
| | imino)]erythromycin A |
| 60. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(3H-imidazo[4,5- |
| | b]pyridin-3-yl)-butyl)-imino)]erythromycin A |
| 61. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(5-nitroindol-1-yl)- |
| | butyl)-imino)]erythromycin A |
| 62. | 2-α-Fluoro-5-O-(3′-N-desmethyl-3′-N-cyclopropyl)-desosaminyl-11,12-dideoxy- |
| | 3-O-decladinosyl-6-O-methyl-3-oxo-12,11-[oxycarbonyl-((4-(5-nitroindol-1-yl)- |
| | butyl)-imino)]erythromycin A |
| 63. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5-fluoroindol-1-yl)- |
| | butyl)-imino)]erythromycin A |
| 64. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(benzotriazol-1-yl)- |
| | butyl)-imino)]erythromycin A |
| 65. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(2- |
| | methylbenzoimidazol-1-yl)-butyl)-imino)]erythromycin A |
| 66. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(2-ethylbenzoimidazol- |
| | 1-yl)-butyl)-imino)]erythromycin A |
| 67. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(benzotriazol-1-yl)- |
| | butyl)-imino)]erythromycin A |
| 68. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(2-ethylbenzoimidazol- |
| | 1-yl)-butyl)-imino)]erythromycin A |
| 69. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-((4-(pyridin-3-yl)- |
| | imidazol-1-yl)-butyl)-imino)]erythromycin A |
| 70. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(2- |
| | methylbenzoimidazol-1-yl)-butyl)-imino)]erythromycin A |
| 71. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(pyrrolo[2,3-b]pyridin-1- |
| | yl)-butyl)-imino)]erythromycin A |
| 72. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(1H-imidazo[4,5- |
| | b]pyridin-1-yl)-butyl)-imino)]erythromycin A |
| 73. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(3H- |
| | imidazo[4,5-b]pyridin-3-yl)-butyl)-imino)]erythromycin A |
| 74. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(5- |
| | nitroindol-1-yl)-butyl)-imino)]erythromycin A |
| 75. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(2-(pyridin-3-ylmethyl)- |
| | benzoimidazol-1-yl)-butyl)-imino)]erythromycin A |
| 76. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(2-(pyridin-3-yl- |
| | methyl)-benzoimidazol-1-yl)-butyl)-imino)]erythromycin A |
| 77. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(2-phenylimidazol-1-yl)- |
| | butyl)-imino)]erythromycin A |
| 78. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(5-fluoroindol-1-yl)- |
| | butyl)-imino)]erythromycin A |
| 79. | 2-α-Fluoro-5-O-(3′-N-desmethyl-3′-N-allyl)-11,12-dideoxy-3-O-decladinosyl-6- |
| | O-methyl-3-oxo-12,11-[oxycarbonyl-((4-(5,6-dimethyl-benzoimidazol-1-yl)- |
| | butyl)-imino)]erythromycin A |
| 80. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5,6- |
| | dimethylbenzoimidazol-1-yl)-butyl)-imino)]erythromycin A |
| 81. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(3H-imidazo[4,5- |
| | c]pyridin-3-yl)-butyl)-imino)]erythromycin A |
| 82. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(2-methyl-3H- |
| | imidazo[4,5-b]pyridin-3-yl)-butyl)-imino)]erythromycin A |
| 83. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(2-methyl-3H- |
| | imidazo[4,5-b]pyridin-3-yl)-butyl)-imino)]erythromycin A |
| 84. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(1H-imidazo[4,5- |
| | c]pyridin-1-yl)-butyl)-imino)]erythromycin A |
| 85. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(6-amino-9H-purin-9- |
| | yl)-butyl)-imino)]erythromycin A |
| 86. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(2-phenylimidazol-1-yl)- |
| | butyl)-imino)]erythromycin A |
| 87. | 2-α-Fluoro-5-O-(3′-N-desmethyl-3′-N-acetyl)-desosaminyl-11,12-dideoxy-3-O- |
| | decladinosyl-6-O-methyl-3-oxo-12,11-[oxycarbonyl-((4-(benzoimidazol-1-yl)- |
| | butyl)-imino)]erythromycin A |
| 88. | 2-α-Fluoro-5-O-(3′-N-desmethyl-3′-N-cyclopropyl methyl)-desosaminyl-11,12- |
| | dideoxy-3-O-decladinosyl-6-O-methyl-3-oxo-12,11-[oxycarbonyl-((4-(3H- |
| | imidazo[4,5-b]pyridin-3-yl)-butyl)-imino)]erythromycin A |
| 89. | 2-α-Fluoro-5-O-(3′-N-desmethyl-3′-N-cyclopropyl methyl)-desosaminyl-11,12- |
| | dideoxy-3-O-decladinosyl-6-O-methyl-3-oxo-12,11-[oxycarbonyl-((4- |
| | (benzoimidazol-1-yl)-butyl)-imino)]erythromycin A |
| 90. | 2-α-Fluoro-5-O-(3′-N-desmethyl-3′-N-cyclopropyl methyl)-desosaminyl-11,12- |
| | dideoxy-3-O-decladinosyl-6-O-methyl-3-oxo-12,11-[oxycarbonyl-((4- |
| | (pyrrolo[2,3-b]pyridin-1-yl)-butyl)-imino)]erythromycin A, |
| 91. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4- |
| | (pyrrolo[2,3-b]pyridin-1-yl)-butyl)-imino)]erythromycin A |
| 92. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4- |
| | (benzotriazol-1-yl)-butyl)-imino)]erythromycin A |
| 93. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4- |
| | (benzoimidazol-1-yl)-butyl)-imino)]erythromycin A |
| 94. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(4-phenyl- |
| | imidazol-1-yl)-butyl)-imino)]erythromycin A |
| 95. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(2-trifluoromethyl- |
| | benzoimidazol-1-yl)-butyl)-imino)]erythromycin A |
| 96. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4,5-diphenyl-imidazol- |
| | 1-yl)-butyl)-imino)]erythromycin A |
| 97. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(4,5-diphenyl-imidazol- |
| | 1-yl)-butyl)-imino)]erythromycin A |
| 98. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(4-phenyl-imidazol-1- |
| | yl)-butyl)-imino)]erythromycin A |
| 99. | 2-α-Fluoro-5-O-(3′-N-desmethyl-3′-N-cyclopropyl methyl)-desosaminyl-11,12- |
| | dideoxy-3-O-decladinosyl-6-O-methyl-3-oxo-12,11-[oxycarbonyl-((4-(4-phenyl- |
| | imidazol-1-yl)-butyl)-imino)]erythromycin A |
| 100. | 2-α-Fluoro-5-O-(3′-N-desmethyl-3′-N-cyclopropyl methyl)-desosaminyl-11,12- |
| | dideoxy-3-O-decladinosyl-6-O-methyl-3-oxo-12,11-[oxycarbonyl-((4- |
| | (benzotriazol-1-yl)-butyl)-imino)]erythromycin A |
| 101. | 2-α-Fluoro-5-O-(3′-N-desmethyl-3′-N-cyclopropyl methyl)-desosaminyl-11,12- |
| | dideoxy-3-O-decladinosyl-6-O-methyl-3-oxo-12,11-[oxycarbonyl-((4-(4- |
| | (pyridin-3-yl)-imidazol-1-yl)-butyl)-imino)]erythromycin A |
| 102. | 2-α-Fluoro-5-O-(3′-N-desmethyl-3′-N-acetyl)-desosaminyl-11,12-dideoxy-3-O- |
| | decladinosyl-6-O-methyl-3-oxo-12,11-[oxycarbonyl-((4-(pyrrolo[2,3-b]pyridin-1- |
| | yl)-butyl)-imino)]erythromycin A |
| 103. | 2-α-Fluoro-5-O-(3′-N-desmethyl-3′-N-acetyl)-desosaminyl-11,12-dideoxy-3-O- |
| | decladinosyl-6-O-methyl-3-oxo-12,11-[oxycarbonyl-((4-(4-phenyl-imidazol-1- |
| | yl)-butyl)-imino)]erythromycin A |
| 104. | 2-α-Fluoro-5-O-(3′-N-desmethyl-3′-N-acetyl)-desosaminyl-11,12-dideoxy-3-O- |
| | decladinosyl-6-O-methyl-3-oxo-12,11-[oxycarbonyl-((4-(1H-imidazo[4,5- |
| | b]pyridin-1-yl)-butyl)-imino)]erythromycin A |
| 105. | 2-α-Fluoro-5-O-(3′-N-desmethyl-3′-N-acetyl)-desosaminyl-11,12-dideoxy-3-O- |
| | decladinosyl-6-O-methyl-3-oxo-12,11-[oxycarbonyl-((4-(3H-imidazo[4,5- |
| | b]pyridin-3-yl)-butyl)-imino)]erythromycin A |
| 106. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(3,5-diphenyl)-pyrazol- |
| | 1-yl)-butyl)-imino)]erythromycin A |
| 107. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(4-(pyridn- |
| | 3-yl)-imidazol-1-yl)-butyl)-imino)]erythromycin A |
| 108. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(1H- |
| | imidazo[4,5-b]pyridin-1-yl)-butyl)-imino)]erythromycin A |
| 109. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(3,5-diphenyl)-pyrazol- |
| | 1-yl)-butyl)-imino)]erythromycin A |
| 110. | 2-α-Fluoro-5-O-(3′-N-desmethyl-3′-N-cyclopropyl methyl)-desosaminyl-11,12- |
| | dideoxy-3-O-decladinosyl-6-O-methyl-3-oxo-12,11-[oxycarbonyl-((4-(1H- |
| | imidazo[4,5-b] pyridin-1-yl)-butyl)-imino)] erythromycin A |
| 111. | 2-α-Fluoro-5-O-(3′-N-desmethyl-3′-N-acetyl)-desosaminyl-11,12-dideoxy-3-O- |
| | decladinosyl-6-O-methyl-3-oxo-12,11-[oxycarbonyl-((4-(4-(pyridin-3-yl)- |
| | imidazo-1-yl)-butyl)-imino)] erythromycin A |
| 112. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(4-(2,4- |
| | difluoro-phenyl)-imidazol-1-yl)-butyl)-imino)] erythromycin A |
| 113. | 2-α-Fluoro-5-O-(3′-N-desmethyl-3′-N-2-ethyl)-desosaminyl-11,12-dideoxy-3-O- |
| | decladinosyl-6-O-methyl-3-oxo-12,11-[oxycarbonyl-((4-(4-(2,4-difluorophenyl)- |
| | imidazol-1-yl)-butyl)-imino)] erythromycin A |
| 114. | 2-α-Fluoro-5-O-(3′-N-desmethyl-3′-N-2-allyl)-desosaminyl-11,12-dideoxy-3-O- |
| | decladinosyl-6-O-methyl-3-oxo-12,11-[oxycarbonyl-((4-(4-(2,4-difluoro-phenyl)- |
| | imidazol-1-yl)-butyl)-imino)] erythromycin A |
| 115. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(furan-3-yl)-imidazol- |
| | 1-yl)-butyl)-imino)] erythromycin A |
| 116. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(furan-3-yl)-imidazol- |
| | 1-yl)-butyl)-imino)] erythromycin A hydrochloride salt |
| 117. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-((4-phenyl-2H-pyrazol- |
| | 1-yl)-butyl)-imino)] erythromycin A |
| 118. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyridin-3-yl)-1H- |
| | imidazol-1-yl)-pentyl)-imino)] erythromycin A |
| 119. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyridin-4-yl)-1H- |
| | imidazol-1-yl)-butyl)-imino)] erythromycin A |
| 120. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(4-(pyridin-4-yl)-1H- |
| | imidazol-1-yl)-butyl)-imino)] erythromycin A |
| 121. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-phenyl-2H-pyrazol-1- |
| | yl)-butyl)-imino) erythromycin A |
| 122. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(1H-indazol-2-yl)- |
| | butyl)-imino)] erythromycin A |
| 123. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(1H-indazol-2-yl)- |
| | butyl)-imino)] erythromycin A |
| 124. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyridin-3-yl)-2H- |
| | pyrazol-1-yl)-butyl)-imino)] erythromycin A |
| 125. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(1H-indazol-1-yl)- |
| | butyl)-imino)] erythromycin A |
| 126. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyrazin-2-yl)-1H- |
| | imidazol-1-yl)-butyl)-imino)] erythromycin A |
| 127. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(thiophen-3-yl)-1H- |
| | imidazol-1-yl)-butyl)-imino)] erythromycin A |
| 128. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(4-(thiophen-3-yl)-1H- |
| | imidazol-1-yl)-butyl)-imino)] erythromycin A |
| 129. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(quinolin-3-yl)-1H- |
| | imidazol-1-yl)-butyl)-imino)] erythromycin A |
| 130. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(1H-indazol-1-yl)- |
| | butyl)-imino)] erythromycin A |
| 131. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyrimin-5-yl)-1H- |
| | imidazol-1-yl)-butyl)-imino)] erythromycin A |
| 132. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(4-(pyrimin-5-yl)-1H- |
| | imidazol-1-yl)-butyl)-imino)]erythromycin A |
| 133. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(furan-2-yl)-1H- |
| | imidazol-1-yl)-butyl)-imino)] erythromycin A |
| 134. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(thiophen-2-yl)-1H- |
| | imidazol-1-yl)-butyl)-imino)] erythromycin A |
| 135. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(4-chloro-phenyl)- |
| | 1H-imidazol-1-yl)-butyl)-imino)] erythromycin A |
| 136. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(4-(4-chloro-phenyl)- |
| | 1H-imidazol-1-yl)-butyl)-imino)] erythromycin A |
| 137. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyridin-2-yl)-1H- |
| | imidazol-1-yl)-butyl)-imino)] erythromycin A |
| 138. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(4-(pyridin-2-yl)-1H- |
| | imidazol-1-yl)-butyl)-imino)] erythromycin A |
| 139. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((9-(4-amino-butyl)-9H- |
| | purin-6-yl)-imino)] erythromycin A |
| 140. | 2-α-Fluoro-5-O-(3′-N-desmethyl-3′-N-2-allyl)-desosaminyl-11,12-dideoxy-3-O- |
| | decladinosyl-6-O-methyl-3-oxo-12,11-[oxycarbonyl-((4-(4-(pyridin-3-yl)-1H- |
| | pyrazol-1-yl)-butyl)-imino)] erythromycin A |
| 141. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(4-(4- |
| | chloro-phenyl)-1H-imidazol-1-yl)-butyl)-imino)] erythromycin A |
| 142. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(4- |
| | (pyridin-2-yl)-1H-imidazol-1-yl)-butyl)-imino)] erythromycin A |
| 143. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(3H-imidazo[4,5- |
| | b]pyridine-2-yl)-butyl)-imino)] erythromycin A |
| 144. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(tetrazol-1-yl)- |
| | imidazol-1-yl)-butyl)-imino)] erythromycin A |
| 145. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(1H-benzoimidazol-2- |
| | yl)-butyl)-imino)] erythromycin A |
| 146. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(6-fluoro-pyridin-3- |
| | yl)-imidazol-1-yl)-butyl)-imino)] erythromycin A |
| 147. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(3H-imidazo[4,5- |
| | c]pyridine-2-yl)-butyl)-imino)] erythromycin A |
| 148. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-([1,2,4]trizol-1-yl)- |
| | phenyl)-butyl)-imino)] erythromycin A |
| 149. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(imidazol-1-yl)- |
| | phenyl)-butyl)-imino)] erythromycin A |
| 150. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(6-chloro-pyridin-3- |
| | yl)-imidazol-1-yl)-butyl)-imino)] erythromycin A |
| 151. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(3-(1-(4-aminobutyl)-1H- |
| | imidazol-4-yl)-phenyl)imino] erythromycin A |
| 152. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyridin-3-yl)-thiazol- |
| | 2-yl)-butyl)-imino)] erythromycin A |
| 153. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(3-(2-(4-aminobutyl)- |
| | thiazol-4-yl)-phenyl)imino] erythromycin A |
| 154. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyrazol-1-yl)- |
| | imidazol-1-yl)-butyl)-imino)] erythromycin A |
| 155. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-([1,4′]-bipyrazolyl-1′- |
| | yl)-butyl)-imino)] erythromycin A |
| 156. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(imidazol-1-yl)- |
| | pyrazol-1-yl)-butyl)-imino)] erythromycin A |
| 157. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyrazol-1-yl)- |
| | imidazol-1-yl)-butyl)-imino)] erythromycin A |
| 158. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-([3,3′]bithiophenyl-5-yl)- |
| | butyl)-imino)] erythromycin A |
| 159. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-([2,3′]bithiophenyl-5′- |
| | yl)-butyl)-imino)] erythromycin A |
| 160. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(furan-2-yl)-thiophen- |
| | 2-yl)-butyl)-imino)] erythromycin A |
| 161. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(oxazol-5-yl)- |
| | imidazol-1-yl)-butyl)-imino)] erythromycin A |
| 162. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(3-(pyrrol-1-yl)- |
| | [1,2,4]triazol-1-yl)-butyl)-imino)] erythromycin A |
| 163. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5-(thiophen-2-yl)- |
| | tetrazol-2-yl)-butyl)-imino)] erythromycin A |
| 164. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(thiophen-3-yl)- |
| | pyrazol-1-yl)-butyl)-imino)] erythromycin A |
| 165. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(furan-3-yl)-pyrazol- |
| | 1-yl)-butyl)-imino)] erythromycin A |
| 166. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(furan-2-yl)-pyrazol- |
| | 1-yl)-butyl)-imino)] erythromycin A |
| 167. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5-phenyl-tetrazol-2-yl)- |
| | butyl)-imino)] erythromycin A |
| 168. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5-(4-methoxy-phenyl)- |
| | tetrazol-2-yl)-butyl)-imino)] erythromycin A |
| 169. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5-(furan-3-yl)-imidazol- |
| | 1-yl)-butyl)-imino)] erythromycin A |
| 170. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(6-pyrazol-1-yl-pyridin- |
| | 3-yl)-butyl)-imino)] erythromycin A |
| 171. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyridin-3-yl)- |
| | pyrazol-1-yl)-butyl)-imino)] erythromycin A |
| 172. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5-thiophen-2-yl- |
| | pyridin-3-yl)-butyl)-imino)] erythromycin A |
| 173. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-phenyl-thiophen-2- |
| | yl)-butyl)-imino)] erythromycin A |
| 174. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(N-(4-amino-butyl)-N- |
| | thiazol-2-yl-nicotinimido)]erythromycin A |
| 175. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(N-(4-amino-butyl)-N- |
| | thiazol-2-yl-nicotinimido)]erythromycin A |
| 176. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(6-pyrrol-1-yl- |
| | pyridin-3-yl)-imidazol-1-yl)-butyl)-imino)] erythromycin A |
| 177. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(6-pyrrol-1-yl-pyridin-3- |
| | yl)-butyl)-imino)] erythromycin A |
| 178. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(2-pyrrol-1-yl-thiazol-5- |
| | yl)-butyl)-imino)] erythromycin A |
| 179. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(6-imidazol-1-yl- |
| | pyridin-3-yl)-butyl)-imino)] erythromycin A |
| 180. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(tetrahydro-furan-2- |
| | yl)-pyridin-3-yl]-butyl)imino] erythromycin A |
| 181. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(4-tolyl-imidazol-1-yl)-butyl)- |
| | imino)] erythromycin A |
| 182. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(4-aminophenyl)-imidazol-1-yl)- |
| | butyl)-imino)] erythromycin A |
| 183. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(4-methyl-3-aminophenyl)-imidazol- |
| | 1-yl)-butyl)-imino)] erythromycin A |
| 184. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(3-(imidazol-1yl)-phenyl)imidazol-1- |
| | yl)-butyl)-imino)] erythromycin A |
| 185. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(3-(N,N-dicyclopropyl-amino)- |
| | phenyl)-imidazol-1-yl)-butyl)-imino)] erythromycin A |
| 186. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(3-(N,N-dimethyl-amino)phenyl)- |
| | imidazol-1-yl)-butyl)-imino)] erythromycin A |
| 187. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(3-(tetrazol-1yl)-phenyl)-imidazol-1- |
| | yl)-butyl)-imino)] erythromycin A |
| 188. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(2-(pyrrol-1yl)-thiozol-5-yl)- |
| | imidazol-1-yl)-butyl)-imino)] erythromycin A |
| 189. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(3-trifluoromethyl-phenyl)-imidazol- |
| | 1-yl)-butyl)-imino)] erythromycin A |
| 190. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(N-(thiazol-2-yl)-benzimido)-butyl)- |
| | imino)] erythromycin A |
| 191. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(2-amino-pyrimidin-5-yl)-imidazol- |
| | 1-yl)-butyl)-imino)] erythromycin A |
| 192. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(4-(pyrrol-1yl)-phenyl)-imidazol-1- |
| | yl)-butyl)-imino)] erythromycin A |
| 193. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(3-(pyrrol-1yl)-phenyl)-imidazol-1- |
| | yl)-butyl)-imino)] erythromycin A |
| 194. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(3-(N-acetyl-amino)-phenyl)- |
| | imidazol-1-yl)-butyl)-imino)] erythromycin A |
| 195. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(3-(N-isopropyl-amino)-phenyl)- |
| | imidazol-1-yl)-butyl)-imino)] erythromycin A |
| 196. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(3-nitrophenyl)-imidazol-1-yl)- |
| | butyl)-imino)] erythromycin A |
| 197. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(3-(N-benzoyl-amino)-phenyl)- |
| | imidazol-1-yl)-butyl)-imino)] erythromycin A |
| 198. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(3,4-dimethyl-phenyl)-imidazol-1- |
| | yl)-butyl)-imino)] erythromycin A |
| 199. | 5-O-(3′-N-desmethyl-3′-N-ethyl)-11,12-dideoxy-3-O-decladinosyl-6-O-methyl- |
| | 3-oxo-12,11-[oxycarbonyl-((4-(4-Pyridin-3-yl-thiazol-2-yl)-butyl)-imino)] |
| | erythromycin A |
| 200. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(4-fluorophenyl)-imidazol-1-yl)- |
| | butyl)-imino)] erythromycin A |
| 201. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(4-methoxy-phenyl)-imidazol-1-yl)- |
| | butyl)-imino)] erythromycin A |
| 202. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(N-(benzthiazol-2-yl)-benzimido)- |
| | butyl)-imino)] erythromycin A |
| 203. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(N-(thiazol-2-yl)-nicotinamido)-butyl)- |
| | imino)] erythromycin A |
| 204. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((4-(4-(3-(N-methyl-amino)-phenyl)- |
| | imidazol-1-yl)-butyl)-imino)] erythromycin A |
| 205. | 2-α-Fluoro-5-O-(3′-N-desmethyl-3′-N-ethyl)desosaminyl-11,12-dideoxy-3-O- |
| | decladinosyl-6-O-methyl-3-oxo-12,11-[oxycarbonyl-((4-(4-(2-aminopyridin-4- |
| | yl)-imidazol-1-yl)-butyl)-imino)] erythromycin A |
| 206. | 2-α-Fluoro-5-O-(3′-N-desmethyl-3′-N-ethyl)desosaminyl-11,12-dideoxy-3-O- |
| | decladinosyl-6-O-methyl-3-oxo-12,11-[oxycarbonyl-((4-(4-(2-aminopyridin-5- |
| | yl)-2H-tetrazol-5-yl)-butyl)-imino)] erythromycin A |
| 207. | 2-α-Fluoro-5-O-(3′-N-desmethyl-3′-N-ethyl)desosaminyl-11,12-dideoxy-3-O- |
| | decladinosyl-6-O-methyl-3-oxo-12,11-[oxycarbonyl-((4-(4-(3,4-difluorophenyl)- |
| | imidazol-1-yl)-butyl)-imino)] erythromycin A |
| 208. | 2-α-Fluoro-5-O-(3′-N-desmethyl-3′-N-ethyl)desosaminyl-11,12-dideoxy-3-O- |
| | decladinosyl-6-O-methyl-3-oxo-12,11-[oxycarbonyl-((4-(4-(2-chloropyridin-4- |
| | yl)-imidazol-1-yl)-butyl)-imino)] erythromycin A |
| 209. | 2-α-Fluoro-5-O-(3′-N-desmethyl-3′-N-ethyl)desosaminyl-11,12-dideoxy-3-O- |
| | decladinosyl-6-O-methyl-3-oxo-12,11-[oxycarbonyl-((4-(4--(6-aminopyridin-3- |
| | yl)-imidazol-1-yl)-butyl)-imino)] erythromycin A |
| 210. | 2-α-Fluoro-5-O-(3′-N-desmethyl-3′-N-ethyl)desosaminyl-11,12-dideoxy-3-O- |
| | decladinosyl-6-O-methyl-3-oxo-12,11-[oxycarbonyl-((4-(5-(2-Chloro-pyridin-4- |
| | yl)-tetrazol-2-yl])-butyl)-imino)] erythromycin A, |
| 211. | 11,12-Dideoxy-3-O-decladinosyl-3-O-(2-nitrophenyl acetyl)-5-O-(3′-N- |
| | desmethyl-3′-N-ethyl)-6-O-methyl-12,11-[oxycarbonyl-((3-imidazol-1-yl)- |
| | propyl)-imino]erythromycin A |
| 212. | 11,12-Dideoxy-3-O-decladinosyl-3-O-(2-nitrophenyl acetyl)-5-O-(3′-N- |
| | desmethyl-3′-N-ethyl)-6-O-methyl-12,11-[oxycarbonyl-((4-imidazol-1-yl)-butyl)- |
| | imino]erythromycin A |
| 213. | 11,12-Dideoxy-3-O-decladinosyl-3-O-(2-nitrophenyl acetyl)-5-O-(3′-N- |
| | desmethyl-3′-N-allyl)-6-O-methyl-12,11-[oxycarbonyl-((3-imidazol-1-yl)- |
| | propyl)-imino]erythromycin A |
| 214. | 11,12-Dideoxy-3-O-decladinosyl-3-O-(2-nitrophenyl acetyl)-5-O-(3′-N- |
| | desmethyl-3′-N-ethyl)-6-O-methyl-12,11-[oxycarbonyl-((4-(3H)-imidazo(4,5-b)- |
| | pyridin-3-yl)-butyl)-imino]erythromycin A |
| 215. | 11,12-Dideoxy-3-O-decladinosyl-3-O-(2-nitrophenyl acetyl)-5-O-(3′-N- |
| | desmethyl-3′-N-ethyl)-6-O-methyl-12,11-[oxycarbonyl-((4-benzimidazol-1-yl)- |
| | butyl)-imino]erythromycin A |
| 216. | 11,12-Dideoxy-3-O-decladinosyl-3-O-(3-nitrophenyl acetyl)-5-O-(3′-N- |
| | desmethyl-3′-N-allyl)-6-O-methyl-12,11-[oxycarbonyl-((4-benzimidazol-1-yl)- |
| | butyl)-imino]erythromycin A |
| 217. | 11,12-Dideoxy-3-O-decladinosyl-3-O-(2-nitrophenyl acetyl)-5-O-(3′-N- |
| | desmethyl-3′-N-allyl)-6-O-methyl-12,11-[oxycarbonyl-(prop-2-en-yl)- |
| | imino]erythromycin A |
| 218. | 11,12-Dideoxy-3-O-decladinosyl-3-O-(4-pyridyl acetyl)-5-O-(3′-N-desmethyl-3′- |
| | N-ethyl)-6-O-methyl-12,11-[oxycarbonyl-((4-imidazol-1-yl)-butyl)- |
| | imino]erythromycin A |
| 219. | 11,12-Dideoxy-3-O-decladinosyl-3-O-(2-pyridyl acetyl)-5-O-(3′-N-desmethyl-3′- |
| | N-ethyl)-6-O-methyl-12,11-[oxycarbonyl-((4-imidazol-1-yl)-butyl)- |
| | imino]erythromycin A |
| 220. | 11,12-Dideoxy-3-O-decladinosyl-3-O-(2-pyridyl acetyl)-5-O-(3′-N-desmethyl-3′- |
| | N-ethyl)-6-O-methyl-12,11-[oxycarbonyl-((4-benzimidazol-1-yl)-butyl)- |
| | imino]erythromycin A |
| 221. | 11,12-Dideoxy-3-O-decladinosyl-3-O-(4-pyridyl acetyl)-5-O-(3′-N-desmethyl-3′- |
| | N-ethyl)-6-O-methyl-12,11-[oxycarbonyl-((4-benzimidazol-1-yl)-butyl)- |
| | imino]erythromycin A |
| 222. | 11,12-Dideoxy-3-O-decladinosyl-3-O-(2-pyridyl acetyl)-5-O-(3′-N-desmethyl-3′- |
| | N-ethyl)-6-O-methyl-12,11-[oxycarbonyl-((4-(3H)-imidazol-[4,5-b]pyridin-3-yl)- |
| | butyl)-imino]erythromycin A |
| 223. | 11,12-Dideoxy-3-O-decladinosyl-3-O-(2-pyridyl acetyl)-5-O-(3′-N-desmethyl-3′- |
| | N-ethyl)-6-O-methyl-12,11-[oxycarbonyl-((4-(1H)-imidazol-[4,5-b]pyridin-1-yl)- |
| | butyl)-imino]erythromycin A |
| 224. | 11,12-Dideoxy-3-O-decladinosyl-3-O-(2-pyridyl acetyl)-5-O-(3′-N-desmethyl-3′- |
| | N-ethyl)-6-O-methyl-12,11-[oxycarbonyl-((4-pyrrolo-[2,3-b]pyridin-1-yl)-butyl)- |
| | imino]erythromycin A |
| 225. | 11,12-Dideoxy-3-O-decladinosyl-3-O-(2-pyridyl acetyl)-5-O-(3′-N-desmethyl-3′- |
| | N-ethyl)-6-O-methyl-12,11-[oxycarbonyl-((4-indol-1-yl)-butyl)- |
| | imino]erythromycin A |
| 226. | 11,12-Dideoxy-3-O-decladinosyl-3-O-(2-pyridyl acetyl)-5-O-(3′-N-desmethyl-3′- |
| | N-ethyl)-6-O-methyl-12,11-[oxycarbonyl-((4-(4-phenyl)-imidazol-1-yl)-butyl)- |
| | imino]erythromycin A |
| 227. | 11,12-Dideoxy-3-O-decladinosyl-3-O-(3-pyridyl acetyl)-5-O-(3′-N-desmethyl-3′- |
| | N-ethyl)-6-O-methyl-12,11-[oxycarbonyl-((4-(1H)-imidazol-[4,5-b]pyridin-1-yl)- |
| | butyl)-imino]erythromycin A |
| 228. | 11,12-Dideoxy-3-O-decladinosyl-3-O-(3-pyridyl acetyl)-5-O-(3′-N-desmethyl-3′- |
| | N-ethyl)-6-O-methyl-12,11-[oxycarbonyl-((4-indol-1-yl)-butyl)- |
| | imino]erythromycin A |
| 229. | 11,12-Dideoxy-3-O-decladinosyl-3-O-(3-pyridyl acetyl)-5-O-(3′-N-desmethyl-3′- |
| | N-ethyl)-6-O-methyl-12,11-[oxycarbonyl-((4-benzimidazol-1-yl)-butyl)- |
| | imino]erythromycin A |
| 230. | 11,12-Dideoxy-3-O-decladinosyl-3-O-(3-pyridyl acetyl)-5-O-(3′-N-desmethyl-3′- |
| | N-ethyl)-6-O-methyl-12,11-[oxycarbonyl-((4-pyrrolo-[2,3-b]pyridin-1-yl)-butyl)- |
| | imino]erythromycin A |
| 231. | 11,12-Dideoxy-3-O-decladinosyl-3-O-(3-pyridyl acetyl)-5-O-(3′-N-desmethyl-3′- |
| | N-ethyl)-6-O-methyl-12,11-[oxycarbonyl-((4-(3H)-imidazol-[4,5-b]pyridin-3-yl)- |
| | butyl)-imino]erythromycin A |
| 232. | 11,12-Dideoxy-3-O-decladinosyl-3-O-(3-pyridylacetyl)-5-O-(3′-N-desmethyl-3′- |
| | N-ethyl)-6-O-methyl-12,11-[oxycarbonyl-((4-(4-phenyl)-imidazol-1-yl)-butyl)- |
| | imino]erythromycin A |
| 233. | 11,12-dideoxy-3-O-decladinosyl-3-O-(2-pyridylacetyl)-5-O-(3′-N-desmethyl-3′- |
| | N-ethyl)-6-O-methyl-12,11-[oxycarbonyl-((N¹-methyl-N¹-pyridin-4-ylmethyl)-2- |
| | aminoethyl)-imino]erythromycin A |
| 234. | 11,12-dideoxy-3-O-decladinosyl-3-O-(2-pyridylacetyl)-5-O-(3′-N-desmethyl-3′- |
| | N-ethyl)-6-O-methyl-12,11-[oxycarbonyl-((N¹-methyl-N¹-pyridin-2-ylmethyl)-2- |
| | aminoethyl)-imino]erythromycin A |
| 235. | 11,12-dideoxy-3-O-decladinosyl-3-O-(2-pyridylacetyl)-5-O-(3′-N-desmethyl-3′- |
| | N-ethyl)-6-O-methyl-12,11-[oxycarbonyl-((N¹-methyl-N¹-pyridin-3-ylmethyl)-2- |
| | aminoethyl)-imino]erythromycin A |
| 236. | 11,12-dideoxy-3-O-decladinosyl-3-O-(2-pyridylacetyl)-5-O-(3′-N-desmethyl-3′- |
| | N-ethyl)-6-O-methyl-12,11-[oxycarbonyl-((N¹-methyl-N¹-quinolin-4-ylmethyl)- |
| | 2-aminoethyl)-imino]erythromycin A |
| 237. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-pyrrolo[2,3-b]pyridin-1-yl)-butyl)- |
| | imino]erythromycin A |
| 238. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-(1H)imidazo[4,5-b]pyridin-1-yl)- |
| | butyl)-imino]erythromycin A |
| 239. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-(3H)imidazo[4,5-b]pyridin-3-yl)- |
| | butyl)-imino]erythromycin A |
| 240. | 11,12-dideoxy-3-O-decladinosyl-3-O-(3-pyridylacetyl)-5-O-(3′-N-desmethyl-3′- |
| | N-ethyl)-6-O-methyl-12,11-[oxycarbonyl-((4-(2-methyl)-benzoimidazo-1-yl)- |
| | butyl)-imino]erythromycin A |
| 241. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-(4-phenyl)-imidazol-1-yl)-butyl)- |
| | imino]erythromycin A |
| 242. | 11,12-dideoxy-3-O-decladinosyl-3-O-(3-pyridylacetyl)-5-O-(3′-N-desmethyl-3′- |
| | N-ethyl)-6-O-methyl-12,11-[oxycarbonyl-((4-pyridin-2-yl)-butyl)- |
| | imino]erythromycin A |
| 243. | 11,12-dideoxy-3-O-decladinosyl-3-O-(3-pyridylacetyl)-5-O-(3′-N-desmethyl-3′- |
| | N-ethyl)-6-O-methyl-12,11-[oxycarbonyl-((4-pyridin-4-yl)-butyl)- |
| | imino]erythromycin A |
| 244. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-benzoimidazo-1-yl)-butyl)- |
| | imino]erythromycin A |
| 245. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-(4-pyridin-3-yl)-imidazol-1-yl)- |
| | butyl)-imino]erythromycin A |
| 246. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-benzotriazol-1-yl)-butyl)- |
| | imino]erythromycin A |
| 247. | 11,12-dideoxy-3-O-decladinosyl-3-O-(4-pyridylacetyl)-5-O-(3′-N-desmethyl-3′- |
| | N-ethyl)-6-O-methyl-12,11-[oxycarbonyl-((4-(4-pyridin-3-yl)-imidazol-1-yl)- |
| | butyl)-imino]erythromycin A |
| 248. | 11,12-dideoxy-3-O-decladinosyl-3-O-(3-pyridylacetyl)-5-O-(3′-N-desmethyl-3′- |
| | N-ethyl)-6-O-methyl-12,11-[oxycarbonyl-((4-(4-pyridin-3-yl)-imidazol-1-yl)- |
| | butyl)-imino]erythromycin A |
| 249. | 11,12-dideoxy-3-O-decladinosyl-3-O-(2-pyridylacetyl)-5-O-(3′-N-desmethyl-3′- |
| | N-ethyl)-6-O-methyl-12,11-[oxycarbonyl-((4-(4-pyridin-3-yl)-imidazol-1-yl)- |
| | butyl)-imino]erythromycin A |
| 250. | 11,12-dideoxy-3-O-decladinosyl-3-O-(4-pyridylacetyl)-5-O-(3′-N-desmethyl-3′- |
| | N-ethyl)-6-O-methyl-12,11-[oxycarbonyl-((4-pyrrolo[2,3-b]pyridin-1-yl)-butyl)- |
| | imino]erythromycin A |
| 251. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-(3H)imidazo[4,5-b]pyridin-3-yl)- |
| | butyl)-imino]erythromycin A |
| 252. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((9-(4-amino-butyl)9H-purin-6-yl)- |
| | imino]erythromycin A |
| 253. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-benzoimidazo-1-yl)-butyl)- |
| | imino]erythromycin A |
| 254. | 11,12-dideoxy-3-O-decladinosyl-3-O-(4-pyridylacetyl)-5-O-(3′-N-desmethyl-3′- |
| | N-ethyl)-6-O-methyl-12,11-[oxycarbonyl-((4-(4-phenyl)-imidazol-1-yl)-butyl)- |
| | imino]erythromycin A |
| 255. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-(4-phenyl)-imidazol-1-yl)-butyl)- |
| | imino]erythromycin A |
| 256. | 11,12-dideoxy-3-O-decladinosyl-3-O-(4-pyridylacetyl)-5-O-(3′-N-desmethyl-3′- |
| | N-ethyl)-6-O-methyl-12,11-[oxycarbonyl-((4-(3H)imidazo[4,5-b]pyridin-3-yl)- |
| | butyl)-imino]erythromycin A |
| 257. | 11,12-dideoxy-3-O-decladinosyl-3-O-(4-pyridylacetyl)-5-O-(3′-N-desmethyl-3′- |
| | N-ethyl)-6-O-methyl-12,11-[oxycarbonyl-((4-benzotriazol-1-yl)-butyl)- |
| | imino]erythromycin A |
| 258. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-(4-pyridin-3-yl)-imidazol-1-yl)- |
| | butyl)-imino]erythromycin A |
| 259. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-pyrrolo[2,3-b]pyridin-1-yl)-butyl)- |
| | imino]erythromycin A |
| 260. | 11,12-dideoxy-3-O-decladinosyl-3-O-(2-pyridylacetyl)-5-O-(3′-N-desmethyl-3′- |
| | N-propargyl)-6-O-methyl-12,11-[oxycarbonyl-((4-benzoimidazo-1-yl)-butyl)- |
| | imino]erythromycin A |
| 261. | 11,12-dideoxy-3-O-decladinosyl-3-O-(2-pyridylacetyl)-5-O-(3′-N-desmethyl-3′- |
| | N-ethyl)-6-O-methyl-12,11-[oxycarbonyl-((4-benzotriazol-1-yl)-butyl)- |
| | imino]erythromycin A |
| 262. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-(1H)imidazo[4,5-b]pyridin-1-yl)- |
| | butyl)-imino]erythromycin A |
| 263. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-benzotriazol-1-yl)-butyl)- |
| | imino]erythromycin A |
| 264. | 11,12-dideoxy-3-O-decladinosyl-3-O-(3-pyridylacetyl)-5-O-(3′-N-desmethyl-3′- |
| | N-ethyl)-6-O-methyl-12,11-[oxycarbonyl-((4-benzotriazol-1-yl)-butyl)- |
| | imino]erythromycin A |
| 265. | 11,12-dideoxy-3-O-decladinosyl-3-O-(2-pyridylacetyl)-5-O-(3′-N-desmethyl-3′- |
| | N-propargyl)-6-O-methyl-12,11-[oxycarbonyl-((4-pyrrolo[2,3-b]pyridin-1-yl)- |
| | butyl)-imino]erythromycin A |
| 266. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-(4-furan-2-yl)-imidazol-1-yl)- |
| | butyl)-imino]erythromycin A |
| 267. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-(4-thiophen-2-yl)-imidazol-1-yl)- |
| | butyl)-imino]erythromycin A |
| 268. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(3-(3[4-(6-amino-9H-purin-9- |
| | yl)propyl)hydrazo)] erythromycin A |
| 269. | 11,12-dideoxy-3-O-decladinosyl-3-O-(2-pyridyl acetyl)-5-O-(3′-N-desmethyl-3′- |
| | N-ethyl)-6-O-methyl-12,11-[oxycarbonyl-((4-(quinolin-8-yl)-butyl)- |
| | imino)]erythromycin A |
| 270. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(quinolin-8-yl)-butyl)- |
| | imino)]erythromycin A |
| 271. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(quinolin-4-yl)-butyl)- |
| | imino)]erythromycin A |
| 272. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(1-methyl-1H-pyrrolo[2,3- |
| | b]pyridin-3-yl)-butyl)-imino)]erythromycin A |
| 273. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(6-amino-9H-purin-9-yl butylimino)] |
| | erythromycin A |
| 274. | 11,12-dideoxy-3-O-decladinosyl-3-O-(3-pyridyl acetyl)-5-O-(3′-N-desmethyl-3′- |
| | N-ethyl)-6-O-methyl-12,11-[oxycarbonyl-(4-(6-amino-9H-purin-9-yl |
| | butylimino)] erythromycin A |
| 275. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(isoquinolin-5-yl)-butyl)- |
| | imino)]erythromycin A |
| 276. | 11,12-dideoxy-3-O-decladinosyl-3-O-(2-pyridyl acetyl)-5-O-(3′-N-desmethyl-3′- |
| | N-ethyl)-6-O-methyl-12,11-[oxycarbonyl-((4-(isoquinolin-5-yl)-butyl)- |
| | imino)]erythromycin A |
| 277. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(4-thiophen-3-yl-1H-imidazol-1-yl)- |
| | butyl)-imino)]erythromycin A |
| 278. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(4-thiophen-3-yl-1H-imidazol-1-yl)- |
| | butyl)-imino)]erythromycin A hydrochloride salt |
| 279. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(6-dimethylamino-9H-purin-9-yl |
| | butylimino)]erythromycin A |
| 280. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(6-pyrrol-1-yl-purin-9-yl)-butyl)- |
| | imino)] erythromycin A |
| 281. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(6-diethylamino-9H-purin-9-yl |
| | butylimino)] erythromycin A |
| 282. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(6-ethylamino-9H-purin-9-yl |
| | butylimino)] erythromycin A |
| 283. | 11,12-dideoxy-3-O-decladinosyl-3-O-(2-pyridyl acetyl)-5-O-(3′-N-desmethyl-3′- |
| | N-ethyl)-6-O-methyl-12,11-[oxycarbonyl-(4-(6-dimethylamino-9H-purin-9-yl |
| | butylimino)] erythromycin A |
| 284. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(6-dimethylamino-9H-purin-9-yl |
| | butylimino)]erythromycin A |
| 285. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(6-methylamino-9H-purin-9-yl |
| | butylimino)]erythromycin A |
| 286. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(6-methylamino-9H-purin-9-yl |
| | butylimino)]erythromycin A |
| 287. | 11,12-dideoxy-3-O-decladinosyl-3-O-(2-pyridyl acetyl)-5-O-(3′-N-desmethyl-3′- |
| | N-2-fluoroethyl)-6-O-methyl-12,11-[oxycarbonyl-((4-(3H-imidazo[4,5-b]pyridin- |
| | 3-yl)-butyl)-imino)]erythromycin A |
| 288. | 11,12-dideoxy-3-O-decladinosyl-3-O-(2-pyridyl acetyl)-5-O-(3′-N-desmethyl-3′- |
| | N-2-fluoroethyl)-6-O-methyl-12,11-[oxycarbonyl-((4-(1H-imidazo[4,5-b]pyridin- |
| | 1-yl)-butyl)-imino)]erythromycin A |
| 289. | 11,12-dideoxy-3-O-decladinosyl-3-O-(2-pyridyl acetyl)-5-O-(3′-N-desmethyl-3′- |
| | N-2-fluoroethyl)-6-O-methyl-12,11-[oxycarbonyl-(4-(6-amino-9H-purin-9-yl |
| | butylimino)] erythromycin A |
| 290. | 11,12-dideoxy-3-O-decladinosyl-3-O-(3-pyridyl acetyl)-5-O-(3′-N-desmethyl-3′- |
| | N-2-fluoroethyl)-6-O-methyl-12,11-[oxycarbonyl-(4-(6-amino-9H-purin-9-yl |
| | butylimino)] erythromycin A |
| 291. | 11,12-dideoxy-3-O-decladinosyl-3-O-(3-pyridyl acetyl)-5-O-(3′-N-desmethyl-3′- |
| | N-2-fluoroethyl)-6-O-methyl-12,11-[oxycarbonyl-((4-(1H-imidazo[4,5-b]pyridin- |
| | 1-yl)-butyl)-imino)] erythromycin A |
| 292. | 11,12-dideoxy-3-O-decladinosyl-3-O-(3-pyridyl acetyl)-5-O-(3′-N-desmethyl-3′- |
| | N-2-fluoroethyl)-6-O-methyl-12,11-[oxycarbonyl-((4-(3H-imidazo[4,5-b]pyridin- |
| | 3-yl)-butyl)-imino)] erythromycin A |
| 293. | 11,12-dideoxy-3-O-decladinosyl-3-O-(3-pyridyl acetyl)-5-O-(3′-N-desmethyl-3′- |
| | N-ethyl)-6-O-methyl-12,11-[oxycarbonyl-(4-(6-dimethylamino-9H-purin-9-yl |
| | butylimino)] erythromycin A |
| 294. | 11,12-dideoxy-3-O-decladinosyl-3-O-(3-pyridyl acetyl)-5-O-(3′-N-desmethyl-3′- |
| | N-ethyl)-6-O-methyl-12,11-[oxycarbonyl-(4-(6-methylamino-9H-purin-9-yl |
| | butylimino)] erythromycin A |
| 295. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-((4-(purin-9-yl)-butyl)- |
| | imino)]erythromycin A |
| 296. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(purin-9-yl)-butyl)- |
| | imino)]erythromycin A |
| 297. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(1H-imidazo[4,5-c]pyridin-1-yl)- |
| | butyl)-imino)]erythromycin A |
| 298. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-((4-(3H-imidazo[4,5-c]pyridin-3-yl)- |
| | butyl)-imino)]erythromycin A |
| 299. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(4-thiophen-2-yl-1H-imidazol-1yl)- |
| | butyl)-imino)]erythromycin A |
| 300. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-((4-(1H-imidazol[4,5-c]pyridin-1-yl)- |
| | butyl)-imino)]erythromycin A |
| 301. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(3H-imidazol[4,5-c]pyridin-3-yl)- |
| | butyl)-imino)]erythromycin A |
| 302. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-((4-(isoquinolin-5-yl)-butyl)- |
| | imino)]erythromycin A |
| 303. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(4-oxazol-5-yl-1H-imidazol-1yl)- |
| | butyl)-butyl)-imino)]erythromycin A |
| 304. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-((4-(4-furan-2-yl-1H-imidazol-1yl)- |
| | butyl)-imino)] erythromycin A |
| 305. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-((4-(4-furan-2-yl-1H-imidazol-1yl)- |
| | butyl)-imino)] erythromycin A hydrochloride salt |
| 306. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-((4-(4-furan-3-yl-1H-imidazol-1yl)- |
| | butyl)-imino)]erythromycin A |
| 307. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(4-furan-3-yl-1H-imidazol-1yl)- |
| | butyl)-imino)]erythromycin A |
| 308. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(thiophen-2-yl)-pyrazol-1-yl)- |
| | butylimino)] erythromycin A |
| 309. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-(furan-2-yl)-pyrazol-1-yl)- |
| | butylimino)] erythromycin A |
| 310. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(furan-2-yl)-pyrazol-1-yl)- |
| | butylimino)] erythromycin A |
| 311. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-(thiophen-2-yl)-1H-pyrazol-1-yl)- |
| | butylimino)] erythromycin A |
| 312. | 11,12-dideoxy-3-O-decladinosyl-3-O-(2-pyridyl acetyl)-5-O-(3′-N-desmethyl-3′- |
| | N-ethyl)-6-O-methyl-12,11-[oxycarbonyl-(4-(6-amino-9H-purin-9-yl |
| | butylimino)]erythromycin A |
| 313. | 11,12-dideoxy-3-O-decladinosyl-3-O-(3-pyridyl acetyl)-5-O-(3′-N-desmethyl-3′- |
| | N-ethyl)-6-O-methyl-12,11-[oxycarbonyl-((4-(quinolin-3-yl)-butyl)- |
| | imino)]erythromycin A |
| 314. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(quinolin-3-yl)-butyl)- |
| | imino)]erythromycin A |
| 315. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(2-thiazolyl)-imidazol-1-yl)- |
| | butylimino)] erythromycin A |
| 316. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-(thiazol-2-yl)-imidazol-1-yl)- |
| | butylimino)] erythromycin A |
| 317. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(furan-3-yl)-pyrazol-1-yl)- |
| | butylimino)] erythromycin A |
| 318. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(thiophen-3-yl)-pyrazol-1-yl)- |
| | butylimino)] erythromycin A |
| 319. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-(thiophen-3-yl)-pyrazol-1-yl)- |
| | butylimino)] erythromycin A |
| 320. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-(furan-3-yl)-pyrazol-1-yl)- |
| | butylimino)] erythromycin A |
| 321. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((3-(4-pyridin-3-yl-1H-imidazol-1-yl)- |
| | propyl)-hydrazo)]erythromycin A |
| 322. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(3-benzoimidazol-1-yl)-propyl)- |
| | hydrazo)] erythromycin A |
| 323. | (R or S) 11,12-dideoxy-3-O-decladinosyl-3-O-(2-pyridyl acetyl)-5-O-(3′-N- |
| | desmethyl-3′-N-allyl)-6-O-methyl-12,11-[oxycarbonyl-((3-(4-pyridin-3-yl-1H- |
| | imidazol-1yl)-propyl)-hydrazo)]erythromycin A |
| 324. | (S or R) 11,12-dideoxy-3-O-decladinosyl-3-O-(2-pyridyl acetyl)-5-O-(3′-N- |
| | desmethyl-3′-N-allyl)-6-O-methyl-12,11-[oxycarbonyl-((3-(4-pyridin-3-yl-1H- |
| | imidazol-1-yl)-propyl)-hydrazo)]erythromycin A |
| 325. | 11,12-dideoxy-3-O-decladinosyl-3-O-(3-pyridyl acetyl)-5-O-(3′-N-desmethyl-3′- |
| | N-ethyl)-6-O-methyl-12,11-[oxycarbonyl-(3-(4-pyridin-3-yl-1H-imidazol-1-yl)- |
| | propyl)-hydrazo)]erythromycin A |
| 326. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(3-(4-pyridin-3-yl-1H-imidazol-1yl)- |
| | propyl)-hydrazo)]erythromycin A |
| 327. | 11,12-dideoxy-3-O-decladinosyl-3-O-(2-pyridyl acetyl)-5-O-(3′-N-desmethyl-3′- |
| | N-ethyl)-6-O-methyl-12,11-[oxycarbonyl-(3-(4-pyridin-3-yl-1H-imidazol-1yl)- |
| | propyl)-hydrazo)]erythromycin A |
| 328. | 11,12-dideoxy-3-O-decladinosyl-3-O-(2-pyridyl acetyl)-5-O-(3′-N-desmethyl-3′- |
| | N-ethyl)-6-O-methyl-12,11-[oxycarbonyl-(3-(3-(6-amino-9H-purin-9-yl- |
| | propyl)hydrazo)]erythromycin A |
| 329. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(3-(4-(thiophen-3-yl)-1H-imidazol-1yl)- |
| | propyl)-hydrazo)]erythromycin A |
| 330. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(3-isoquinolin-5-yl)-propyl)- |
| | hydrazo)]erythromycin A |
| 331. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(3-purin-9-yl)-propyl)- |
| | hydrazo)]erythromycin A |
| 332. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((3-purin-9-yl)-propyl)- |
| | hydrazo)]erythromycin A |
| 333. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(3-(4-(furan-2-yl)-1H-imidazol-1- |
| | yl)propyl)-hydrazo)]erythromycin A |
| 334. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(3-(3-(6-amino-9H-purin-9-yl- |
| | propyl)hydrazo)] erythromycin A |
| 335. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(3-(4-(furan-3-yl)-1H-imidazol-1- |
| | yl)propyl)-hydrazo)]erythromycin A |
| 336. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(3-(4-(furan-3-yl)-1H-imidazol-1- |
| | yl)propyl)-hydrazo)]erythromycin A |
| 337. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(3-(3-(6-amino-9H-purin-9-yl- |
| | propyl)hydrazo)]erythromycin A |
| 338. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(3-(4-(thiophen-2-yl-1H-imidazol-1-yl)- |
| | propyl)-hydrazo)]erythromycin A |
| 339. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(3-(4-(thiophen-2-yl-1H-imidazol-1-yl)- |
| | propyl)-hydrazo)]erythromycin A |
| 340. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(3-(4-(thiophen-3-yl-1H-imidazol-1-yl)- |
| | propyl)-hydrazo)]erythromycin A |
| 341. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(3-(imidazo[4,5-b]pyridin-1-yl)- |
| | propyl)-hydrazo)] erythromycin A |
| 342. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(3-(imidazo[4,5-b]pyridin-3-yl)- |
| | propyl)-hydrazo)] erythromycin A |
| 343. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(3-(4-(thiazol-2-yl-1H-imidazol-1-yl)- |
| | propyl)-hydrazo)]erythromycin A |
| 344. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(3-(4-(thiazol-2-yl-1H-imidazol-1-yl)- |
| | propyl)-hydrazo)]erythromycin A |
| 345. | (R or S)11,12-dideoxy-3-O-decladinosyl-3-O-(2-pyridyl acetyl)-5-O-(3′-N- |
| | desmethyl-3′-N-allyl)-6-O-methyl-12,11-[oxycarbonyl-hydrazo]erythromycin A |
| 346. | (S or R) 11,12-dideoxy-3-O-decladinosyl-3-O-(2-pyridyl acetyl)-5-O-(3′-N- |
| | desmethyl-3′-N-allyl)-6-O-methyl-12,11-[oxycarbonyl-hydrazo]erythromycin A |
| 347. | 11,12-dideoxy-3-O-decladinosyl-3-O-(3-pyridyl acetyl)-5-O-(3′-N-desmethyl-3′- |
| | N-ethyl)-6-O-methyl-12,11-[oxycarbonyl-hydrazo)]erythromycin A |
| 348. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((3-(4-pyridin-3-yl-1H-imidazol-1-yl)- |
| | propyl)-hydrazo)]erythromycin A, 9-(O-methyl)oxime |
| 349. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-hydrazo]erythromycin A, 9-(O- |
| | methyl)oxime |
| 350. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-((4-(4-(6-fluoro-pyridin-3-yl)-1H- |
| | imidazol-1-yl)-butyl)-imino)]erythromycin A |
| 351. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(4-(6-fluoro-pyridin-3-yl)-1H- |
| | imidazol-1-yl)-butyl)-imino)]erythromycin A |
| 352. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(4-(1H-imidazol-1-yl)phenyl)- |
| | butyl)-imino)]erythromycin A |
| 353. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-((4-(5-(3-aminophenyl)-thiazol-2-yl)- |
| | butyl)-imino)]erythromycin A |
| 354. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(2-amino-pyrimidin)-5-yl)- |
| | imidazol-1-yl)-butylimino)] erythromycin A |
| 355. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(2-aminopyridin-4-yl)-imidazol- |
| | 1-yl)-butylimino)] erythromycin A |
| 356. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(6-aminopyridin-3-yl)-imidazol- |
| | 1-yl)-butylimino)] erythromycin A |
| 357. | 11,12-dideoxy-3-O-decladinosyl-3-O-(3-pyridyl acetyl)-5-O-(3′-N-desmethyl-3′- |
| | N-ethyl)-6-O-methyl-12,11-[oxycarbonyl-(4-(6-(dimethylamino)-9H-purin-9-yl)- |
| | butylimino)] erythromycin A |
| 358. | 11,12-dideoxy-3-O-decladinosyl-3-O-(2-nitrophenyl acetyl)-5-O-(3′-N- |
| | desmethyl-3′-N-allyl)-6-O-methyl-12,11-[oxycarbonyl-(3-(5-phenyl-tetrazol-1- |
| | yl)propylimino)] erythromycin A |
| 359. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-pyridin-3-yl-pyrazol-1-yl)- |
| | butylimino)] erythromycin A |
| 360. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(2-methyl-2H-tetrazol-5- |
| | yl)phenyl)butylimino)] erythromycin A |
| 361. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-(3-thienyl)-imidazol-1- |
| | yl)butylimino)] erythromycin A |
| 362. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(5-methyl-[1,3,4]-oxadiazol-2- |
| | yl)phenyl)butylimino)] erythromycin A |
| 363. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-([2,3′]-bithiophenyl-5- |
| | yl)butylimino)] erythromycin A |
| 364. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-([2,3′]-bithiophenyl5-yl)butylimino)] |
| | erythromycin A |
| 365. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-[3,3′]-bithiophenyl-5-yl)butylimino)] |
| | erythromycin A |
| 366. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-[3,3′]-bithiophenyl-5-yl)butylimino)] |
| | erythromycin A |
| 367. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(5-pyridin-3-yl)-tetrazol-2- |
| | yl)butylimino)] erythromycin A |
| 368. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(5-(furan-2-yl)-imidazol-1- |
| | yl)butylimino)] erythromycin A |
| 369. | 11,12-dideoxy-3-O-decladinosyl-3-O-(2-methylpropanoyl)-5-O-(3′-N-desmethyl- |
| | 3′-N-allyl)-6-O-methyl-12,11-[oxycarbonyl4-(4-(4-(furan-2-yl)-imidazol-1-yl)- |
| | butylimino)] erythromycin A |
| 370. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(4-methoxy-phenyl)-imidazol-1- |
| | yl)-butylimino)] erythromycin A |
| 371. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-(4-methoxy-phenyl)-imidazol-1- |
| | yl)-butylimino)] erythromycin A |
| 372. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-(2-methoxy-phenyl)-imidazol-1- |
| | yl)-butylimino)] erythromycin A |
| 373. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(2-methoxy-phenyl)-1H-imidazol- |
| | 1-yl)-butylimino)] erythromycin A |
| 374. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-phenyl-thiophen-2-yl)- |
| | butylimino)] erythromycin A |
| 375. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-phenyl-thiophen-2-yl)- |
| | butylimino)] erythromycin A |
| 376. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-furan-2-yl-thiophen-2- |
| | yl)butylimino)] erythromycin A |
| 377. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-furan-2-yl-thiophen-2-yl)- |
| | butylimino)] erythromycin A |
| 378. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(5-(4-methoxyphenyl)-tetrazol-2-yl)- |
| | butylimino)] erythromycin A |
| 379. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(5-(4-methoxyphenyl)-tetrazol-2-yl)- |
| | butylimino)] erythromycin A |
| 380. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(5-phenyl-tetrazol-2-yl)-butylimino)] |
| | erythromycin A |
| 381. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(5-pyridin-4-yl-tetrazol-2-yl)- |
| | butylimino)] erythromycin A |
| 382. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(5-pyridin-4-yl-tetrazol-2-yl)- |
| | butylimino)] erythromycin A |
| 383. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(5-phenyl-tetrazol-2-yl)-butylimino)] |
| | erythromycin A |
| 384. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(6-chloropyridin-3-yl)-imidazol- |
| | 1-yl)butylimino)] erythromycin A |
| 385. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-3((3-(4-(imidazol-1-yl)-pyrazol-1-yl)- |
| | propyl)-hydrazo)] erythromycin A |
| 386. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(3-(3-(4-pyrazol-1-yl]-imidazol-1-yl)- |
| | propyl)-hydrazo)] erythromycin A |
| 387. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-(3-aminophenyl)-imidazol-1-yl)- |
| | butylimino)] erythromycin A |
| 388. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-3-((3-([1,4′]-bipyrazol-1′-yl)-propyl)- |
| | hydrazo)] erythromycin A |
| 389. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(3-aminophenyl)-imidazol-1-yl)- |
| | butylimino)] erythromycin A |
| 390. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-pyrimidin-5-yl-imidazol-1- |
| | yl)butylimino)] erythromycin A |
| 391. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(3-(3-(4-pyrimidin-2-yl-imidazol-1-yl)- |
| | propyl)-hydrazo] erythromycin A |
| 392. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-pyrimidin-5-yl-imidazol-1- |
| | yl)butylimino)] erythromycin A |
| 393. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(5-(furan-2-yl)-tetrazol-2- |
| | yl]butylimino)] erythromycin A |
| 394. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(5-(thiophen-2-yl)-tetrazol-2-yl])- |
| | butylimino)] erythromycin A |
| 395. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(5-(furan-2-yl)-tetrazol-1-yl]- |
| | butylimino)] erythromycin A |
| 396. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(5-(furan-2-yl)-tetrazol-1-yl]- |
| | butylimino)] erythromycin A |
| 397. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(5-(furan-2-yl)-tetrazol-2- |
| | yl]butylimino)] erythromycin A |
| 398. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(5-pyridin-3-yl-tetrazol-2-yl)- |
| | butylimino)] erythromycin A |
| 399. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(5-pyridin-2-yl-tetrazol-2-yl)- |
| | butylimino)] erythromycin A |
| 400. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(5-pyridin-2-yl-tetrazol-2-yl)- |
| | butylimino)] erythromycin A |
| 401. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(5-thiophen-2-yl-tetrazol-1-yl)- |
| | butylimino)] erythromycin A |
| 402. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(5-thiophen-2-yl-tetrazol-1-yl)- |
| | butylimino)] erythromycin A |
| 403. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(5-(thiophen-2-yl-tetrazol-2-yl)- |
| | butylimino)] erythromycin A |
| 404. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(1H-benzimidazol-2-yl)-butylimino)] |
| | erythromycin A |
| 405. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(1H-imidazol[4,5-b]pyridin-2-yl)- |
| | butylimino)] erythromycin A |
| 406. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(1H-imidazol[4,5-b]pyridin-2-yl)- |
| | butylimino)] erythromycin A |
| 407. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-tetrazol-1-yl)-imidazol-1-yl)- |
| | butylimino)] erythromycin A |
| 408. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-tetrazol-1-yl)-imidazol-1-yl)- |
| | butylimino)] erythromycin A |
| 409. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(1H-benzimidazol-2-yl)-butylimino)] |
| | erythromycin A |
| 410. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(3-({3-[4-(6-fluoro-pyridin-3-yl- |
| | imidazol-1-yl] propyl}hydrazo] erythromycin A |
| 411. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(3-({3-[4-(6-fluoro-pyridin-3-yl- |
| | imidazol-1-yl] propyl}hydrazo] erythromycin A |
| 412. | 11,12-dideoxy-3-O-decladinosyl-3-O-(2-methyl propanoyl)-5-O-(3′-N- |
| | desmethyl-3′-N-allyl)-6-O-methyl-12,11-[oxycarbonyl-(4-(benzimidazol-1-yl)- |
| | butylimino)] erythromycin A |
| 413. | 11,12-dideoxy-3-O-decladinosyl-3-O-(2-methyl propanoyl)-5-O-(3′-N- |
| | desmethyl-3′-N-allyl)-6-O-methyl-12,11-[oxycarbonyl-(4-(4-(3-pyridyl)- |
| | imidazol-1-yl)-butylimino)] erythromycin A |
| 414. | 11,12-dideoxy-3-O-decladinosyl-3-O-(2-methyl propanoyl)-5-O-(3′-N- |
| | desmethyl-3′-N-allyl)-6-O-methyl-12,11-[oxycarbonyl-(4-(4-imidazol[4,5- |
| | b]pyridin-1-yl)-butylimino)] erythromycin A |
| 415. | 11,12-dideoxy-3-O-decladinosyl-3-O-(2-methyl propanoyl)-5-O-(3′-N- |
| | desmethyl-3′-N-allyl)-6-O-methyl-12,11-[oxycarbonyl-(4-(6-amino-9H-purin-9- |
| | yl)butylimino)] erythromycin A |
| 416. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-([1,4′]-bipyrazol-1′-yl)butylimino] |
| | erythromycin A |
| 417. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-[4-(pyrazol-1-yl)-imidazol-1- |
| | yl)butylimino] erythromycin A |
| 418. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-([1,4′]-biimidazol-1′-yl) butylimino)] |
| | erythromycin A |
| 419. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-([1,4′]-biimidazol-1′-yl)butylimino)] |
| | erythromycin A |
| 420. | 11,12-dideoxy-3-O-decladinosyl-3-O-(2-methylpropanoyl)-5-O-(3′-N-desmethyl- |
| | 3′-N-allyl)-6-O-methyl-12,11-[oxycarbonyl-(4-(imidazo[4,5-b]pyridin-3- |
| | yl)butylimino)] erythromycin A |
| 421. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-pyrazol-1-yl)-imidazol-1- |
| | yl)butylimino)] erythromycin A |
| 422. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(1H-imidazo[4,5-c]pyridin-2- |
| | yl)butylimino)] erythromycin A |
| 423. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-(1H-imidazo[4,5-c]pyridin-2- |
| | yl)butylimino)] erythromycin A |
| 424. | 11,12-dideoxy-3-O-decladinosyl-3-O-(2-methylpropanoyl)-5-O-(3′-N-desmethyl- |
| | 3′-N-allyl)-6-O-methyl-12,11-[oxycarbonyl-(4-(4-(6-fluoro-pyridin-3-yl)- |
| | imidazol-1-yl)butylimino)] erythromycin A |
| 425. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-phenyl-thiazol-2-yl)butylimino)] |
| | erythromycin A |
| 426. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-phenyl-thiazol-2-yl)butylimino)] |
| | erythromycin A |
| 427. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(3-((3-(4-pyrimidin-2-yl)-imidazol-2- |
| | yl)propyl)-hydrazo)] erythromycin A |
| 428. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-3-((3-(4-pyrimidin-5-yl)-imidazol-2- |
| | yl)propyl)-hydrazo)] erythromycin A |
| 429. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-[4-(1H-[1,2,4]-triazol-1- |
| | yl)phenyl]butylimino)] erythromycin A |
| 430. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-[4-(1H-[1,2,4]-triazol-1- |
| | yl)phenyl]butylimino)] erythromycin A |
| 431. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-pyrimidin-5-yl- |
| | phenyl)butylimino)] erythromycin A |
| 432. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(3-(3-(4-pyrimidin-5-yl-imidazol-1- |
| | yl)propyl)hydrazo)] erythromycin A |
| 433. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(3-({3-[4-(imidazol-1-yl)-pyrazol-1- |
| | yl]propyl}hydrazo)] erythromycin A |
| 434. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(3-{[3-([1,4′]-biimidazol-1′- |
| | yl)propyl}hydrazo)] erythromycin A |
| 435. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(3-(3-(4-pyrazol-1-yl)-imidazol-1-yl)- |
| | propyl)-hydrazo] erythromycin A |
| 436. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-3-((3-(1H-imidazo[4,5-b]pyridine-1-yl)- |
| | propyl)-hydrazo)] erythromycin A |
| 437. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-(oxazol-5-yl)-imidazol-1- |
| | yl)butylimino)] erythromycin A |
| 438. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-imidazol-1-yl)-pyrazol-1- |
| | yl]butylimino)] erythromycin A |
| 439. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-imidazol-1-yl)-pyrazol-1- |
| | yl]butylimino)] erythromycin A |
| 440. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-(2H-tetrazol-5-yl)- |
| | phenyl)butylimino)] erythromycin A |
| 441. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-[4-(2H-tetrazol-5-yl)- |
| | phenyl]butylimino)] erythromycin A |
| 442. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-(4-(4-(6-chloropyridin-3-yl)-imidazol- |
| | 1-yl]butylimino)] erythromycin A |
| 443. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-3-((3-([1,4′]-bipyrazol-1-yl)-)-propyl)- |
| | hydrazo)] erythromycin A |
| 444. | 11,12-dideoxy-3-O-decladinosyl-3-O-(2-nitrophenyl acetyl)-5-O-(3′-N- |
| | desmethyl-3′-N-allyl)-6-O-methyl-12,11-[oxycarbonyl-(4-benzimidazol-1- |
| | yl)butylimino)] erythromycin A |
| 445. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-(4-(4-(6-fluoro-pyridin-3-yl)-imidazol- |
| | 1-yl)-butylimino)] erythromycin A |
| 446. | 5-O-(3′-N-desmethyl-3′-N-ethyl)desosaminyl-11,12-dideoxy-3-O-decladinosyl-6- |
| | O-methyl-3-oxo-12,11-[oxycarbonyl-(4-(4-(2-amino-pyrimidin)-5-yl)-pyrazol-1- |
| | yl l)-butyl)-imino)] erythromycin A |
| 447. | 5-O-(3′-N-desmethyl-3′-N-ethyl)desosaminyl-11,12-dideoxy-3-O-decladinosyl-6- |
| | O-methyl-3-oxo-12,11-[oxycarbonyl-((4-(4-tetrazol-1-yl-imidazol-1-yl)-butyl)- |
| | imino)] erythromycin A |
| 448. | 5-O-(3′-N-desmethyl-3′-N-ethyl)desosaminyl-11,12-dideoxy-3-O-decladinosyl-6- |
| | O-methyl-3-oxo-12,11-[oxycarbonyl-(4-(4-(3-(N-acetyl-amino)-phenyl)- |
| | imidazol-1-yl)-butyl)-imino)] erythromycin A |
| 449. | 5-O-(3′-N-desmethyl-3′-N-ethyl)desosaminyl-11,12-dideoxy-3-O-decladinosyl-6- |
| | O-methyl-3-oxo-12,11-[oxycarbonyl-(4-(4-(4-fluoro-phenyl)-imidazol-1-yl)- |
| | butyl)-imino)] erythromycin A |
| 450. | 5-O-(3′-N-desmethyl-3′-N-ethyl)desosaminyl-11,12-dideoxy-3-O-decladinosyl-6- |
| | O-methyl-3-oxo-12,11-[oxycarbonyl-((4-(4-(3-(N,N-dimethyl-amino)-phenyl)- |
| | imidazol-1-yl)-butyl)-imino)] erythromycin A |
| 451. | 5-O-(3′-N-desmethyl-3′-N-ethyl)desosaminyl-11,12-dideoxy-3-O-decladinosyl-6- |
| | O-methyl-3-oxo-12,11-[oxycarbonyl-(4-(4-(4-methoxy-phenyl)-imidazol-1-yl)- |
| | butyl)-imino)] erythromycin A |
| 452. | 5-O-(3′-N-desmethyl-3′-N-ethyl)desosaminyl-11,12-dideoxy-3-O-decladinosyl-6- |
| | O-methyl-3-oxo-12,11-[oxycarbonyl-(4-(4-(2-amino-pyridin)-5-yl)-imidazol-1-yl |
| | l)-butyl)-imino)] erythromycin A |
| 453. | 5-O-(3′-N-desmethyl-3′-N-ethyl)desosaminyl-11,12-dideoxy-3-O-decladinosyl-6- |
| | O-methyl-3-oxo-12,11-[oxycarbonyl-(4-(4-(N-(benzthiazol-2-yl)-benzamido)- |
| | butyl)-imino)] erythromycin A |
| 454. | 5-O-(3′-N-desmethyl-3′-N-ethyl)desosaminyl-11,12-dideoxy-3-O-decladinosyl-6- |
| | O-methyl-3-oxo-12,11-[oxycarbonyl-((4-(4-furan-3-yl-imidazol-1-yl)-butyl)- |
| | imino)] erythromycin A |
| 455. | 5-O-(3′-N-desmethyl-3′-N-ethyl)desosaminyl-11,12-dideoxy-3-O-decladinosyl-6- |
| | O-methyl-3-oxo-12,11-[oxycarbonyl-((4-(4-furan-2-yl-imidazol-1-yl)-butyl)- |
| | imino)] erythromycin A |
| 456. | 5-O-(3′-N-desmethyl-3′-N-ethyl)desosaminyl-11,12-dideoxy-3-O-decladinosyl-6- |
| | O-methyl-3-oxo-12,11-[oxycarbonyl-(4-(4-(3-(N-isopropyl-amino)-phenyl)- |
| | imidazol-1-yl)-butyl)-imino)] erythromycin A |
| 457. | 5-O-(3′-N-desmethyl-3′-N-ethyl)desosaminyl-11,12-dideoxy-3-O-decladinosyl-6- |
| | O-methyl-3-oxo-12,11-[oxycarbonyl-(4-(4-(N-(thiazol-2-yl)-nicotinamido)- |
| | butyl)-imino)] erythromycin A |
| 458. | 5-O-(3′-N-desmethyl-3′-N-ethyl)desosaminyl-11,12-dideoxy-3-O-decladinosyl-6- |
| | O-methyl-3-oxo-12,11-[oxycarbonyl-((4-(6-imidazol-1-yl-pyridin-3-yl)-butyl)- |
| | imino)] erythromycin A |
| 459. | 5-O-(3′-N-desmethyl-3′-N-ethyl)desosaminyl-11,12-dideoxy-3-O-decladinosyl-6- |
| | O-methyl-3-oxo-12,11-[oxycarbonyl-(4-(4-(N-(thiazol-2-yl)-benzamido)-butyl)- |
| | imino)] erythromycin A |
| 460. | 5-O-(3′-N-desmethyl-3′-N-ethyl)desosaminyl-11,12-dideoxy-3-O-decladinosyl-6- |
| | O-methyl-3-oxo-12,11-[oxycarbonyl-((4-(2-pyrrol-1-yl-thiazol-5-yl)-butyl)- |
| | imino)] erythromycin A |
| 461. | 5-O-(3′-N-desmethyl-3′-N-ethyl)desosaminyl-11,12-dideoxy-3-O-decladinosyl-6- |
| | O-methyl-3-oxo-12,11-[oxycarbonyl-((4-(4-(3-(N,N-dicyclopropyl-amino)- |
| | phenyl)-imidazol-1-yl)-butyl)-imino)] erythromycin A |
| 462. | 5-O-(3′-N-desmethyl-3′-N-ethyl)desosaminyl-11,12-dideoxy-3-O-decladinosyl-6- |
| | O-methyl-3-oxo-12,11-[oxycarbonyl-((4-(4-(2-pyrrol-1-yl-thiazol-5-yl)-imidazol- |
| | 1-yl)-butyl)-imino)] erythromycin A |
| 463. | 5-O-(3′-N-desmethyl-3′-N-ethyl)desosaminyl-11,12-dideoxy-3-O-decladinosyl-6- |
| | O-methyl-3-oxo-12,11-[oxycarbonyl-((4-(4-thiaophen-2-yl-pyridin-3-yl)-butyl)- |
| | imino)] erythromycin A |
| 464. | 11,12-dideoxy-3-O-decladinosyl-6-O-methyl-3-oxo-12,11-[oxycarbonyl-(4-(4-(4- |
| | methyl-3-aminophenyl)-imidazol-1-yl]-butylimino] erythromycin A |
| 465. | 2-Methyl-11,12-dideoxy-3-O-decladinosyl-6-O-methyl-3-oxo-12,11- |
| | [oxycarbonyl-4-(4-(3-amino-4-methylphenyl)-imidazol-1-yl)-butylimino] |
| | erythromycin A |
| 466. | 11,12-dideoxy-3-O-decladinosyl-6-O-methyl-3-oxo-12,11-[oxycarbonyl-(4-(N- |
| | (2-thiazolyl)-nicotinamido)-butylimino] erythromycin A |
| 467. | 2-Methyl-11,12-dideoxy-3-O-decladinosyl-6-O-methyl-3-oxo-12,11- |
| | [oxycarbonyl-(4-(4-Imidazo[4,5-b]pyridin-3-yl)-butylimino] erythromycin A |
| 468. | 2-Methyl-11,12-dideoxy-3-O-decladinosyl-6-O-methyl-3-oxo-12,11- |
| | [oxycarbonyl-4-(4-(4-fluorophenyl)-imidazol-1-yl)-butylimino] erythromycin A |
| 469. | 2-α-Fluoro-5-O-(3′-N-desmethyl-3′-N-ethyl)-11,12-dideoxy-3-O-decladinosyl-6- |
| | O-methyl-3-oxo-12,11-[oxycarbonyl-((3-phenyl-allyl)-hydrazo)]erythromycin A |
| 470. | 2-α-Fluoro-5-O-(3′-N-desmethyl-3′-N-allyl)-11,12-dideoxy-3-O-decladinosyl-6- |
| | O-methyl-3-oxo-12,11-[oxycarbonyl-((3-(4-thiophen-3-yl-imidazol-1-yl)propyl)- |
| | hydrazo)] erythromycin A |
| 471. | 11,12-dideoxy-3-O-decladinosyl-3-O-(3-pyridyl acetyl)-5-O-(3′-N-desmethyl-3′- |
| | N-allyl)-6-O-methyl-12,11-[oxycarbonyl-imino)]erythromycin A |
| 472. | 11,12-dideoxy-3-O-decladinosyl-3-O-(2-pyridyl acetyl)-5-O-(3′-N-desmethyl-3′- |
| | N-allyl)-6-O-methyl-12,11-[oxycarbonyl-imino)]erythromycin A |

Compounds of Formula I can be prepared by using the procedures described in PCT applications WO 2006/080954, WO 2006/046112 and in our copending Indian patent application 1383/DEL/2005 (which has been filed with the PCT with application number PCT/IB2006/051686) and 3138/DEL/2005. Compounds of Formula II can be prepared by using procedures described in PCT applications. WO 2006/013409, WO 2005/030786 and WO 2006/035301.

In addition, some of the compounds described herein (446-464) may also be prepared by the following reaction sequences as depicted in Scheme I below, which has been described in our Indian application and 3138/DEL/2005, entitled “Ketolide derivatives as antibacterial agents”, filed on Nov. 23, 2005 and which has been filed with PCT and has the application number PCT/IB2006/003298.

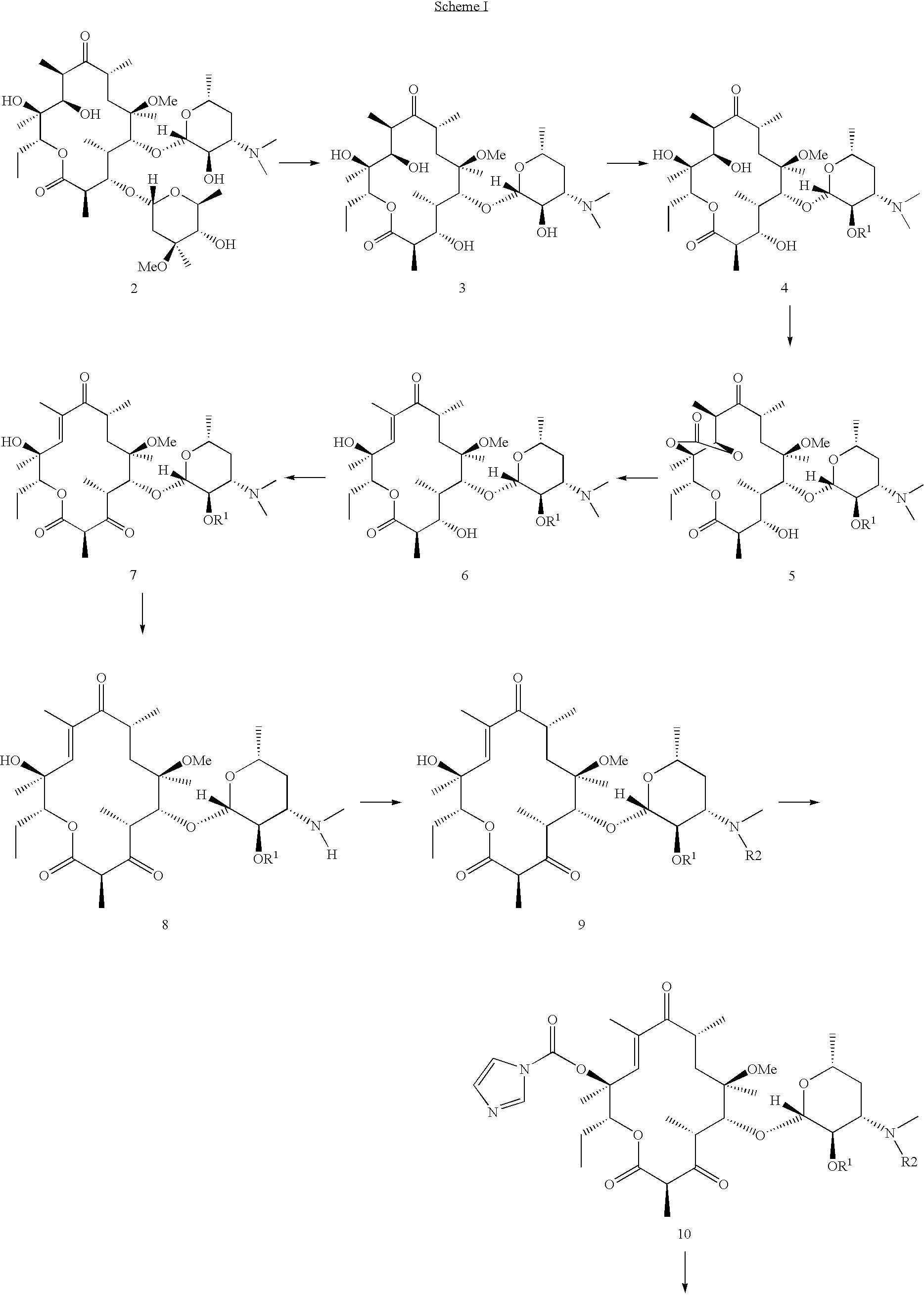

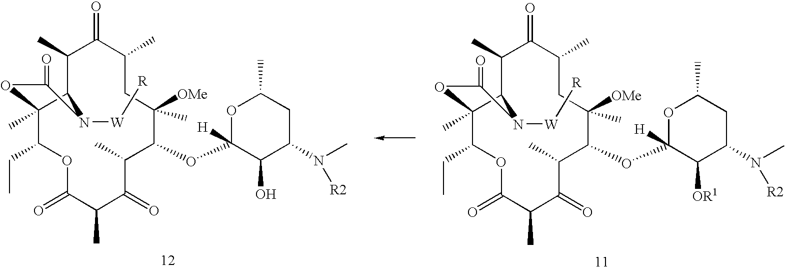

The following examples describe the detailed processes followed in Scheme I.

### EXAMPLE 1

### Preparation of Compound of Formula 3

Clarithromycin (25 gm, 33.4 mmol) was added to aqueous solution of hydrochloric acid at an ambient temperature in portion wise. The reaction mixture was neutralized with solid sodium bicarbonate and the aqueous layer was extracted with ethyl acetate. Organic layer was washed with water, brine, and dried over anhydrous sodium sulphate and the solvent was removed under reduced pressure to afford crude product. The crude product was crystallized from ethyl acetate and hexane mixture.

### EXAMPLE 2

### Preparation of Compound of Formula 4

Benzoic anhydride (2.5 equiv.) followed by triethylamine (6 equiv.) was added to a solution of compound of Formula 2 (1 equiv.) in dichloromethane and stirred at an ambient temperature for about 40 hours. The reaction was quenched by addition of sodium bicarbonate solution. The aqueous layer was extracted with dichloromethane, washed successively with water, brine, and dried over anhydrous sodium sulphate and the solvent was removed under reduced pressure to give crude product. The crude product obtained was crystallized from ethyl acetate and hexane mixture.

### EXAMPLE 3

### Preparation of Compound of Formula 5

Triphosgene (1.5 equiv.) was added to a solution of compound of Formula 3 (1 equiv.) in dichloromethane and pyridine was slowly added (15 equiv.) to it. The reaction mixture was stirred for about 4 hours and the reaction was quenched by addition of ice-cold water. Reaction mixture was diluted with dichloromethane and washed with water followed by brine, dried over anhydrous sodium sulphate and concentrated under reduced pressure to afford the desired product.

### EXAMPLE 4

### Preparation of Compound of Formula 6

Tetramethyl guanidine (2.2 equiv.) was added to a solution of compound of Formula 4 (1 equiv.) in dimethylformamide and heated at about 70° C., stirred for about 10 hours. The reaction mixture was cooled to an ambient temperature. Organic layer was extracted with ethyl acetate and washed with water followed by brine, dried over anhydrous sodium sulphate and concentrated under reduced pressure to obtain the desired product.

### EXAMPLE 5

### Preparation of Compound of Formula 7

Dess-Martine Periodinane (2.5 equiv.) was added to a solution of compound of Formula 5 (1 equiv.) in dichloromethane and refluxed for about an hour. The reaction was cooled to an ambient temperature and quenched by addition of saturated aqueous potassium carbonate solution followed by saturated sodium thiosulphate solution and stirred. Aqueous layer was separated and extracted with dichloromethane. The dichloromethane layer was washed with water, brine, dried over anhydrous sodium sulphate and concentrated under reduced pressure to afford the desired product.

### EXAMPLE 6

### Preparation of compound of Formula 8

N-iodosuccinimide (2 equiv.) was added to a solution of compound of Formula 6 (1 equiv.) in dry acetonitrile:dichloromethane (2:1) at about 0° C. and the reaction mixture was allowed to attain an ambient temperature and stirred. The reaction mixture was stirred with sodium bisulphite solution followed by stirring with sodium carbonate solution. Dichloromethane was evaporated under reduced pressure. The aqueous layer was extracted with ethyl acetate, washed successively with water, brine, and dried over anhydrous sodium sulphate and then the solvent was removed under reduced pressure to afford the desired product.

### EXAMPLE 7

### Preparation of Compound of Formula 9

Solid sodium hydrogen carbonate (5 equiv.) and ethyl iodide (6 equiv.) were added to a solution of compound of Formula 8 (1 equiv.) in acetonitrile under argon at an ambient temperature and stirred for about 24 hours. The reaction was quenched by the addition of water, diluted with ethyl acetate and washed with water followed by brine, dried over anhydrous sodium sulphate and concentrated under reduced pressure to yield a crude product. The crude product was purified by silica gel column chromatography (thoroughly neutralized with triethylamine) using 10-15% acetone in hexane to afford the desired product.

OR (when R² is isopropyl)

Acetone (5 equiv.) and acetic acid were added (2 equiv.) into the solution of compound of Formula 8 (1 equiv.) in methanol and stirred at ambient temperature for 1 hour. Sodiumcyanoborohydride (5 equiv.) was added in portions to it and the reaction mixture was again stirred at ambient temperature for 15 hours. The excess solvent was evaporated to dryness and compound was dissolved into methylene chloride, washed with water followed by brine. The organic layer was dried over anhydrous sodium sulphate, filtered and concentrated to give the solid. The crude product was purified by silica gel column chromatography (thoroughly neutralized with triethylamine) using 10-15% acetone in hexane to afford the desired product. Example 8: Preparation of compound of Formula 10

N,N′-carbonyldiimidazole (3 equiv.) was added to a solution of compound of Formula 8 (1 equiv.) in dimethylformamide:tetrahydrofuran (3:2) at an ambient temperature, cooled, and sodium hydride (3 equiv.) was added in portions and was stirred. The reaction mixture was quenched by addition of water. It was extracted with ethyl acetate. The organic layer was washed with water, brine, dried over anhydrous sodium sulphate and concentrated under reduced pressure to afford the desired product.

### EXAMPLE 9

### Preparation of Compound of Formula 11

The compound of Formula 9 (1 equiv.) and R—W—NH₂ (2 equiv.) were taken in water in acetonitrile and heated at 70° C., stirred for about 20 hours, reaction mixture was cooled to attain an ambient temperature and acetonitrile was removed under reduced pressure. The resulting residue was taken in ethyl acetate and washed with water, brine, dried over anhydrous sodium sulphate, and filtered. The filtrate was collected and concentrated under reduced pressure. The compound was purified by silica gel column chromatography (thoroughly neutralized with triethylamine) using 25-30% acetone in hexane to afford the desired product.

### EXAMPLE 10

### Preparation of Compound of Formula 12

The compound of Formula 10 was taken in methanol and refluxed. The reaction mixture was cooled to attain an ambient temperature and methanol was evaporated under reduced pressure. The compound was purified by silica gel column using 2-10% methanol in dichloromethane to afford the desired product.

Compounds of Formulae I and II described herein displayed antibacterial activity in vitro especially against strains which are resistant to macrolides either due to efflux (mef strains) or ribosomal modification (erm) strains. These compounds are useful in the treatment of community acquired pneumonia, upper and lower respiratory tract infections, skin and soft tissue infections, hospital acquired lung infections, bone and joint infections, and other bacterial infections, for example, mastitis, catether infection, foreign body, prosthesis infections or peptic ulcer disease.

Minimum inhibitory concentration (MIC) has been an indicator of in vitro antibacterial activity widely used in the art.

### Procedure

### Medium

a) Cation adjusted Mueller Hinton Agar (MHA-Difco)

### b) Trypticase Soya Agar (TSA)

### Inoculum Preparation

The cultures were streaked on TSA for aerobic cultures and MHA with 5% sheep blood for fastidious cultures. Aerobic cultures were incubated at 37° C. for about 18-24 hours. Fastidious cultures were incubated CO₂ incubation (5% CO₂) at 37° C. for about 18-24 hours. Three to four well-isolated colonies were taken and saline suspensions were prepared in sterile densimat tubes. The turbidity of the culture was adjusted to 0.5-0.7 Mc Farland standard (1.5×10⁸ CFU/ml). The cultures were diluted 10 fold in saline to get inoculum size of approximately 1-2×10⁷ organisms/ml.

### Preparation of Drug Concentration

1 mg/ml concentration of stock solution of drugs was prepared in dimethylsulfoxide/distilled water/solvent given in National Committee for Clinical Laboratory Standards (NCCLS) manual. Serial two fold dilutions of the compounds and standard drugs were prepared as per NCCLS manual.

Stock solution was changed according to the need of the experiment.

### Preparation of Agar Plates

Two ml of respective drug concentration was added to 18 ml of Molten Mueller Hinton agar to get the required range, for example 0.015 μg/ml-16 μg/ml. For fastidious cultures 1 ml of sheep blood was added in Molten Mueller Hinton agar. For control MHA and MHA with 5% sheep blood plates without antibiotic for each set were prepared. One MHA and MHA with 5% sheep blood plate without antibiotic for determining quality check for media was prepared.

### Preparation of Teflon Template

1 μl of each culture on each plate was replicated with the help of replicator (Denley's multipoint replicator). The spots were allowed to dry and the plates were incubated for about 18-24 hours at 37° C. Fastidious cultures were incubated at 37° C. in CO₂ incubator. The results were noted comparing with the control plates.

### Endpoint Definition

The concentration of drug at which there was complete disappearance of growth spot or formation of less than 10 colonies per spot was considered as Minimum Inhibitory Concentration (MIC).

The MICs of Quality Control (QC) strains were plotted on the QC chart for agar dilution method. If the MICs were within the range, the results interpreted by comparing MICs of standards against all organisms with those of test compounds.

### Precautions & Quality Control Measures

### Quality Control Strains

Staphylococcus aureus ATCC 29213
Enterococcus faecalis ATCC 29212
Eschericia coli ATCC 25922
Pseudomonas aeruginosa ATCC 27853
All 60 cultures were visually checked for purity.

Media Control: NCCLS disc diffusion assay using 10 μg discs of Gentamicin (Difco) against Pseudomonas aeruginosa ATCC 27853. A zone diameter of 16-21 mm was considered for optimum cation (Magnesium and Calcium) content of the media. The diameter was plotted in the media QC chart.

### REFERENCES

National Committee for Clinical Laboratory Standards (NCCLS), Methods for Dilution Antimicrobial Susceptibility Tests for Bacteria That Grow Aerobically Fifth Edition; Approved Standard. M7-A5, Vol. 20. No. 2 (January 2000).
National Committee for Clinical Laboratory Standards, Performance Standards for Antimicrobial Susceptibility Testing—Twelfth informational supplement, M 100-S12, Vol. 22 No. 1 (January 2002).

### Results

a) The Compounds Described Herein Exhibited MIC Values Against Staphylococcus aureus in the range of between about 0.06 μg/mL to about 8 μg/mL and even between about 0.125 μg/mL to about 1 μg/mL.
b) The compounds described herein also exhibited MIC values against Staphylococcus aureus in the range of between about 0.125 μg/mL to about >16 μg/mL, and even between about 0.25 μg/mL to about 4 μg/mL.
c) The compounds described herein exhibited MIC values against sensitive Streptococcus pneumoniae in the range of between about <0.008 μg/mL to about 16 μg/mL and even between about 0.008 μg/mL to about 0.5 μg/mL.
d) The compounds described herein also exhibited MIC values against sensitive Streptococcus pneumoniae in the range of between about 0.015 μg/mL to about 4 μg/mL, and even between about 0.008 μg/mL to about 0.125 μg/mL.
e) The compounds described herein exhibited MIC values against erythromycin resistant Streptococcus pneumoniae in the range of between about 0.06 μg/mL to about >16 μg/mL and even between about 0.125 μg/mL to about 4 μg/mL.
f) The compounds described herein also exhibited MIC values against erythromycin resistant Streptococcus pneumoniae strains in the range of between about 0.008 μg/mL to about >16 μg/mL, and even between about 0.015 μg/mL to about 4 μg/mL.
g) The compounds described herein exhibited MIC values against Haemophilus influenzae in the range of between about <0.03 μg/mL to about >32 μg/mL and even between about <0.06 μg/mL to about 16 μg/mL.
h) The compounds described herein also exhibited MIC values against Haemophilus influenzae in the range of between about <0.03 μg/mL to about >32 μg/mL, and even between about <0.06 μg/mL to about >16 μg/mL.
i) The compounds described herein exhibited MIC values against Moraxella species in the range of between about <0.004 μg/mL to about 4 μg/mL and even between about 0.03 μg/mL to about 1 μg/mL.
j) The compounds described herein also exhibited MIC values against Moraxella catarrhalis in the range of between about <0.015 μg/mL to about >16 μg/mL, and even between about 0.06 μg/mL to about 8 μg/mL.
k) The compounds described herein exhibited MIC values against telithromycin resistant Streptococcus pneumoniae in the range of between about 0.5 μg/mL to about >16 μg/mL and even between about 2 μg/mL to about 16 μg/mL.
l) The compounds described herein also exhibited MIC values against telithromycin resistant Streptococcus pneumoniae strains in the range of between about 0.25 μg/mL to about >16 μg/mL, between about 0.5 μg/mL to about 8 μg/mL.
m) The compounds described herein exhibited MIC values against sensitive Streptococcus pyogenes in the range of between about 0.008 μg/mL to about 1 μg/mL and even between about 0.015 μg/mL to about 0.15 μg/mL.
n) The compounds described herein also exhibited MIC values against sensitive Streptococcus pyogenes strains in the range of between about <0.015 μg/mL to about 2 μg/mL, and even between about 0.008 μg/mL to about 0.25 μg/mL.
o) The compounds described herein exhibited MIC values against erythromycin resistant Streptococcus pyogenes in the range of between about 0.015 μg/mL to about >16 μg/mL and even between about 0.125 μg/mL to about 16 μg/mL.
p) The compounds described herein also exhibited MIC values against erythromycin resistant Streptococcus pyogenes strains in the range of between about <0.015 μg/mL to about >16 μg/mL, and even between about 0.004 μg/mL to about 2 μg/mL.
q) The compounds described herein exhibited MIC values against methicillin resistant Staphylococcus aureus to about >16 μg/mL.
r) The compounds described herein exhibited MIC values against sensitive enterococci species in the range of between about <0.03 μg/mL to about 1 g/mL and even between about 0.03 μg/mL to about 0.5 μg/mL.
s) The compounds described herein exhibited MIC values against resistant enterococci species in the range of between about 1 μg/mL to about >16 μg/mL and even between about 2 μg/mL to about 16 μg/mL.
t) The compounds described herein also exhibited MIC values against Vancomycin resistant Eenterococci in the range of between about 0.125 μg/mL to about >16 μg/mL, and even between about 0.5 μg/mL to about 8 μg/mL.
u) The compounds described herein exhibited MIC values against Helicobacter pylori in the range of between about 0.06 μg/mL to about 0.5 μg/mL.
v) The compounds described herein exhibited MIC values against E. faecalis in the range of between about 0.03 μg/mL to about 4 μg/mL, and even between about 0.06 μg/mL to about 1 μg/mL.

In another aspect, provided herein are methods for treating or preventing acne and inflammatory conditions thereof comprising administering to a mammal therapeutically effective amounts of one or more compounds of Formula I, II or mixture thereof.

Methods may involve one or more of the following embodiments. For example, acne vulgaris can be mild to moderate (e.g., whitehead, blackhead, papules or pimples). The acne vulgaris can also be severe to very severe (e.g., nodules or cysts). Patients with mild acne vulgaris generally have blackheads and whiteheads but pimples are not present. Patients with moderate acne vulgaris generally have blackheads, whiteheads and small pimples, which are confined to the face and inflammation they cause is minimal. Patients with severe acne vulgaris generally have blackheads, whiteheads, and deeper pimples with more pronounced swelling. Very severe acne occurs on the face, neck, shoulders, chest, and back. Postules and deeper cysts occur with very severe acne vulgaris and scarring may result if the condition is not cared properly. The inflammatory conditions as enumerated above may involve redness, blemishes, swelling, heat and pain. In particular, the invention can be used to treat acne vulgaris and condition the skin cells.

In another aspect, provided herein are pharmaceutical compositions comprising therapeutically effective amounts of one or more compounds of Formula I, II or mixture thereof, optionally together with pharmaceutically acceptable carriers, excipients or diluents. The said pharmaceutical composition can be used to prevent or treat acne vulgaris and inflammatory conditions thereof.

In another aspect, provided herein are methods for treating or preventing acne vulgaris and inflammatory conditions thereof comprising administering to a mammal in need thereof therapeutically effective amounts of one or more compounds of Formula I, II or mixture thereof in combination with one or more therapeutic agents selected from alcohol, benzoyl peroxide, clindamycin, tretinoin, vitamin E, vitamin A and its derivatives, tetracycline, isotretinoin, vitamin C, vitamin D, chaparral, dandelion root, licoric root, Echinacea, kelp, cayenine, sassafras, elder flowers, pantothenic acid, para amino benzoic acid, biotin, cholin, inositol, folic acid, calcium, magnesium, potassium, vitamin B₆, zinc, carotenoid, azelaic acid, and other therapeutic agents, which can be used to treat acne vulgaris or condition the skin.

In another aspect, provided herein are methods for treating or preventing acne vulgaris and inflammatory conditions thereof comprising administering to a mammal in need thereof therapeutically effective amounts of combination pharmaceutical composition. The term “combination pharmaceutical composition” as used herein refers to, in one embodiment; a single pharmaceutical composition comprises one or more compounds of Formula I, II or mixture thereof and one or more therapeutic agent selected from alcohol, benzoyl peroxide, clindamycin, tretinoin, vitamin E, vitamin A and its derivatives, tetracycline, isotretinoin, vitamin C, vitamin D, chaparral, dandelion root, licoric root, Echinacea, kelp, cayenine, sassafras, elder flowers, pantothenic acid, para amino benzoic acid, biotin, cholin, inositol, folic acid, calcium, magnesium, potassium, vitamin B₆, zinc, carotenoid, azelaic acid or mixture thereof, together with one or more pharmaceutically acceptable carries, excipients or diluents. The combination pharmaceutical composition, in second embodiment, refers to a first pharmaceutical composition comprising therapeutically effective amount of one or more compounds of Formula I, II or mixture thereof, together with pharmaceutically acceptable carriers, excipients or diluents, a second pharmaceutical composition comprising one or more therapeutic agents selected from alcohol, benzoyl peroxide, clindamycin, tretinoin, vitamin E, vitamin A and its derivatives, tetracycline, isotretinoin, vitamin C, vitamin D, chaparral, dandelion root, licoric root, Echinacea, kelp, cayenine, sassafras, elder flowers, pantothenic acid, para amino benzoic acid, biotin, cholin, inositol, folic acid, calcium, magnesium, potassium, vitamin B₆, zinc, carotenoid, azelaic acid or mixture thereof, together with one or more pharmaceutically acceptable carriers, excipients, diluents or mixture thereof. The separate pharmaceutical compositions can be administered simultaneously, separately or sequentially.

The term “therapeutically effective amount” as used herein refers to the amount that produce desired therapeutic window/response. A person skilled in the art can determine the therapeutically effective amount.

Included within the scope of this invention are pharmaceutically acceptable salts. The pharmaceutically acceptable salts include, for example, alkali metal (e.g. Sodium or potassium) or alkaline earth metal (e.g. calcium or magnesium) salts and addition salts of acids or bases. Suitable pharmaceutically acceptable acid addition salts may be prepared from an inorganic acid or from an organic acid. Example of such inorganic acids include, but not limited to, hydrochloric, hydrobromic, hydroiodic, sulfuric, phosphoric acid and like. Appropriate organic acids include, but not limited to, aliphatic, cycloaliphatic, aromatic, heterocyclic, carboxylic and sulfonic classes of organic acids, such as, formic, acetic, propionic, succinic, glycolic, gluconic, lactic, malic, tartaric, dihydroxytartaric acid, citric, ascorbic, glucuronic, maleic, fumeric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, beta-hydroxybutyric, cyclohexylaminosulfonic, galactaric and galacturonic acid and the like.

Suitable pharmaceutically acceptable base addition salts include, but not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, and procaine and the like. The salt forms differ from the compound described herein in certain physical properties such as solubility in polar solvent, but the salts are otherwise equivalent for purposes of this invention.

The present invention also includes within its scope prodrugs of the agents described herein. In general, such prodrugs will be functional derivatives of these compounds, which are readily convertible in vivo into the required compound. Conventional procedure for the selection and preparation of suitable prodrug derivatives are described, for example, in “design of prodrugs”, H Bundgaard and, Elsevier, 1985. The present invention also includes metabolites, which become active upon introduction into the biological system. Where the compounds according to the invention have at least one chiral center, they may accordingly exist as enantiomers. Where the compounds according to invention possess two or more chiral centers, they may additionally exist as diastereomers. It is to be understood that all such isomers and racemic mixtures therefore are encompassed within the scope of the present invention. Furthermore, some of the crystalline forms for compounds described herein may exist as polymorphs and as such are intended to be included in the present invention. In addition, some of the compounds described herein may form solvates with water (i-e hydrates) or common organic solvents. Such solvates are also encompassed within the scope of this invention.

The composition described herein may be suitable for oral, rectal, parenteral, intravenous, topical, transdermal, subcutaneous, intramuscular, and the like. Although any suitable route of administration may be employed for providing the patient with an effective dosage of the composition according to the methods of the present invention, oral administration is preferred. The composition may be formulated to provide immediate or sustained release of the therapeutic agents. The agents described herein can be administered alone but will generally be administered as an admixture with a suitable “pharmaceutically acceptable carrier”. The term “pharmaceutically acceptable carrier” is intended to include non-toxic, inert solid, semi-solid or liquid filter, diluent, encapsulating material or formulation auxiliary of any type.

Solid form preparation for oral administration may include capsules, tablets, pills, powder, granules and suppository. For solid form preparation, the active compound is mixed with at least one inert, pharmaceutically acceptable excipient or carrier such as sodium citrate, dicalcium phosphate and/or a filter an extender such as starch, lactose, sucrose, glucose, mannitol and silicic acid; binders such as carboxymethyl cellulose, alginates, gelatins, polyvinylpyrrolidinone, sucrose, acacia; disintegrating agents such as agar-agar, calcium carbonate, potato starch, aliginic acid, certain silicates and sodium carbonate; absorption accelators such as quaternary ammonium compounds; wetting agents such as cetyl alcohol, glycerol, monostearate; adsorbents such as kaolin; lubricants such as talc, calcium stearate, magnesium stearate, solid polyethyleneglycol, sodium lauryl sulphate and mixture thereof.

In case of capsules, tablets, pills, the dosage form may also comprise buffering agents. The solid preparation of tablets, capsules, pills, granules can be prepared with coating and shells such as enteric coating and other coatings well known in the pharmaceutical formulating art.

Liquid form preparation for oral administration includes pharmaceutically acceptable emulsions, solution, suspensions, syrups and elixirs. For liquid form preparation active compound is mixtured with water or other solvent, solubilizing agents and emulsifiers such as ethylalcohol, isopropyl alcohol, ethyl carbonate, ethyl acetate, benzyl alcohol, benzyl benzoate, propylene glycol, 1,3-butylene glycol, dimethylformamide, oils (such as cottonseed, groundnut, corn, germ, olive, castor and sesame oil), glycerol and fatty acid ester of sorbitan and mixture thereof.

Besides inert diluents, the oral composition can also include adjuvant such as wetting agents, emulsifying agents, suspending agents, sweetening agents, flavoring agents and perfuming agent.

Injectable preparations such as sterile injections, aqueous or oleaginous suspensions may be formulated according to the art using suitable dispersing or wetting and suspending agents. Among the acceptable vehicles and solvents that may be employed is water, Ringers solution or isotonic sodium chloride.

Dosage form for topical or transdermal administration includes ointments, pastes, creams, lotions, gel, powders, solutions, spray, inhalants or patches. The active compound is admixed under sterile condition with a pharmaceutically acceptable carrier and any needed preservatives or buffer as may be required.

The pharmaceutical preparation is in unit dosage form. In such form, the preparation is subdivided into unit doses containing appropriate quantities of the active component.

The formulation as described herein may be formulated so as to provide quick sustained, or delayed release of the active ingredient after administration to the patient by employing procedure well known to the art. The composition may be administered as a depot formulation that permits sustained release, limits access to general circulation. Such a formulation may be provided as a slow release implant, be microencapsulated, or attached to a biodegradable polymer. The compound is administered in a sustained release formulation as a tablet or capsule. A sustained release formulation is a preparation that releases the active component over a desired period of time after administration. A sustained release formulation is prepared by applying a biodegradable, bioerodible or bioabsorbable polymeric formulation that is compatible on the surface of the active component. Examples of sustained release formulation include, but are not limited to, hydroxypropylmethylcellulose (HPMC), hydrogenated vegetable oil (HVO), ethylcellulose, polyvinylpyrrolidione, pyran copolymer, polyhydroxypropylmethacryl-amidephenol, polyhydroxy-ethylaspartamidephenol, or polyethyleneoxidepolylysin substituted with palmitoyl residues, polylactic acid, polyepsilon caprolactone, polyhydroxy butyric acid, polyorthoesters, polyacetals, polydihydro-pyrans, polycyano acrylates.

The term “biodegradable” means that the polymeric formulation degrades overtime by the action of enzymes, by hydrolytic action and/or by other similar mechanisms in the human body. By “bioerodible” it is meant that the polymeric formulation erodes or degrades over time due, at least in part, to contact with substances found in the surrounding tissue fluids or cellular action. By “bioabsorbable”, it is meant that the polymeric formulation is broken down and absorbed within the body of a mammal, for example, by a cell or tissue. “Biocompitable” means that the polymeric formulation does not cause substantial tissue irritation or necrosis.

The combination pharmaceutical compositions as described herein can be administered together combined in a single dosage form or they can be administered separately, simultaneously or sequentially, each in its dosage form but as part of the same therapeutic treatment program or regimen. Separate administration of each compound, at different times and by different routes, will sometimes be recommended.

The dosage forms described herein can be prepared by conventional methods known to a person of ordinary skill in the art. The dosage of the pharmaceutical composition of the present invention may be appropriately determined with reference to the dosages recommended for the respective active components and can be selected according to the recipient, the age and body weight, current clinical status, administration time, dosage form, method of administration, and combination of the active components, among other factors.

The combination pharmaceutical composition of the present invention can show a marked synergistic effect compared with administration of either active component alone. Furthermore, since the pharmaceutical composition of the present invention develops sufficient efficacy with reduced doses as compared with the administration of any one of the active components alone, the side effects of the respective components can be reduced.

### Pharmacological Testing

Method: Minimum inhibitory concentration (MIC) of compound described herein and standard drugs were determined against clinical and ATCC isolates (n=6) of Propionibacterium acnes [Propionibacterium acnes I (Sensitive), Propionibacterium acnes II (Sensitive), Propionibacterium acnes 6523, Propionibacterium acnes ATCC 6919, Propionibacterium.acnes (clinical isolate) and two isolates of Propionibacterium acnes (Resistant)] as per NCCLS guidelines (M11-A5) by agar dilution method. Brucella agar (Difco) with hemin (5 μg/ml) and vitamin K1 (1 μg/ml) supplemented with 5% (v/v) laked sheep blood was used as media. Stock concentrations of standard drug were prepared in respective diluents as per NCCLS guidelines. Stock solution (1 mg/ml) of NCEs was prepared in DMSO and serial two fold dilutions of drug were prepared and mixed with agar. Inoculum was prepared from freshly grown isolates on brucella blood agar and their turbidity was adjusted to approximately 0.5 McFarland. Replicate 1 1 of cultures on agar plate with the help of a replicator (Denley's multipoint replicator). All plates were incubated at 37° C. for 48 hours in anaerobic jar under anaerobic condition created by using Anaxomat. Results were recorded after incubation by comparing with the drug free control plates.

Results: Macrolides (Erythromycin, Clarithromycin and Azithromycin) showed lower MIC values in the range of 0.06 to 0.5 μg/mL against Propionibacterium acnes 6523, ATCC 6919, Propionibacterium.acnes (clinical isolate), II and I isolates, whereas against resistant P. acnes (NDDR I and II) isolates greater than 16 μg/mL MIC values were observed. All the P. acnes isolate showed resistance to metronidazole. Similarly Clindamycin also showed lower MIC value (0.25 μg/mL) against sensitive P. acnes isolates, but against P. acnes (NDDR I and II) MIC values were 16 μg/mL. Telithromycin was active against the sensitive P. acnes isolates and MIC values were <0.03 mg/mL, whereas against resistant P. acnes isolates (NDDR I and II) moderate activity (2 μg/mL) was observed. Compound described herein were found to be active against isolates of Propionibacterium acnes 6523, Propionibacterium.acnes (clinical isolate), and I, II and ATCC 6919. Compounds described herein showed MIC values in the range of 0.03 to 1 μg/mL. Further, MIC values were in the range of 0.125 to 4 μg/mL against P. acnes (NDDR I) and 0.25 to 4 μg/mL against P. acnes (NDDR II). Some of the compounds described herein showed MIC values in the range of 4 to greater than 16 μg/mL against resistant P. acnes (NDDR I and II) isolates.

1. Use of 14-membered macrolide derivatives for the manufacture of a medicament for treating or preventing acne vulgaris and associated inflammatory conditions, wherein 14-membered macrolide derivatives are selected from one or more compounds of Formula I, II or mixture thereof, 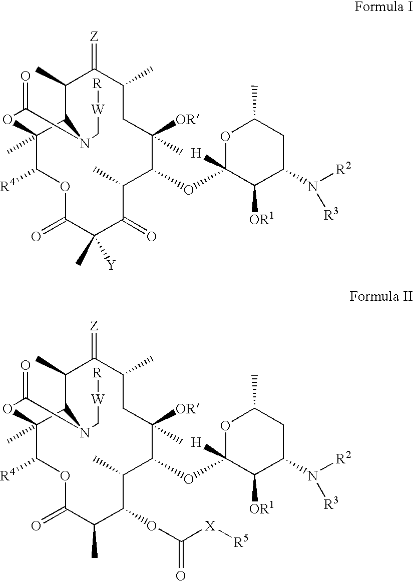
pharmaceutically acceptable salts, pharmaceutically acceptable solvates, enantiomers, diastereomers or polymorphs thereof, wherein:
R¹ is hydrogen or a hydroxyl-protecting group;
R² and R³ are independently hydrogen, alkyl, alkenyl, alkynyl, cycloalkyl, aryl, heterocycle, aralkyl, (heterocycle)alkyl or COR¹¹, wherein R¹¹ is hydrogen, alkyl or aralkyl, with the proviso that R² and R³ can be simultaneously methyl only when Y is hydrogen;
W is alkenyl, -G(CH₂)_{q}J-, —CR⁹R¹⁰, —NR⁹— or —SO₂, wherein
q is an integer of from 2 to 6;
G is no atom, —CO, —CS or —SO₂;
R⁹ and R¹⁰ are independently hydrogen or alkyl; and
J is no atom, —CR⁹R¹⁰ or N(R¹²)(CH₂)ₘ, wherein m is an integer of from 0 to 6; R⁹ and R¹⁰ are the same as defined earlier; and R¹² is hydrogen, alkyl, alkylene, alkynyl, COR⁸ or —(CH₂)ₘ—R⁸ wherein R⁸ is alkyl, aryl or heterocycle;
R is no atom, hydrogen, aryl or heterocycle;
R⁴ is alkyl, alkenyl or alkynyl;
R⁵ is alkyl, aryl or heterocycle;
R′ is alkyl or —(CH₂)ᵣ—U, wherein r is an integer of from 1 to 4 and U is alkenyl or alkynyl;
X is -Q(CH₂)ₖ—, {wherein k is an integer of from 1 to 6, Q is no atom, —NR⁹— or oxygen [wherein R⁹ is hydrogen or alkyl]}, further alkylene chain of -Q(CH₂)ₖ— is optionally substituted with alkyl, hydroxy or alkoxy;
Y is hydrogen, halogen, cyano or alkyl; and
Z is oxygen, sulfur or NOR¹¹, wherein R¹¹ is the same as defined earlier.

2. The use of anyone of the following macrolide derivative for the manufacture of a medicament for treating or preventing acne vulgaris and associated inflammatory conditions.

| Comp. | |
|---|---|
| No. | Nomenclature |
| 1. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((3-(imidazol-1-yl)- |
| | propyl)-imino)]erythromycin A |
| 2. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(1H-imidazo[4,5-b]- |
| | pyridin-1-yl)-butyl)-imino)]erythromycin A |
| 3. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(3H-imidazo[4,5-b]- |
| | pyridin-1-yl)-butyl)-imino)]erythromycin A |
| 4. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(benzoimidazol-1-yl)- |
| | butyl)-imino)]erythromycin A |
| 5. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(imidazol-1-yl)-butyl)- |
| | imino)]erythromycin A |
| 6. | 2-α-Fluoro-5-O-(3′-N-desmethyl-3′-N-cyclopropyl)-desosaminyl-11,12- |
| | dideoxy-3-O-decladinosyl-6-O-methyl-3-oxo-12,11-[oxycarbonyl-((3- |
| | (imidazol-1-yl)-propyl)-imino)]erythromycin A, |
| 7. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((3-(imidazol-1-yl)- |
| | propyl)-imino)]erythromycin A |
| 8. | 2-α-Fluoro-5-O-(3′-N-desmethyl-3′-N-propyl)-desosaminyl-11,12-dideoxy-3-O- |
| | decladinosyl-6-O-methyl-3-oxo-12,11-[oxycarbonyl-((3-(imidazol-1-yl)- |
| | propyl)-imino)]erythromycin A |
| 9. | 2-α-Fluoro-5-O-(3′-N-desmethyl-3′-N-propyl)-desosaminyl-11,12-dideoxy-3-O- |
| | decladinosyl-6-O-methyl-3-oxo-12,11-[oxycarbonyl-((4-(benzoimidazol-1-yl)- |
| | butyl)-imino)]erythromycin A |
| 10. | 2-α-Fluoro-5-O-(3′-N-desmethyl-3′-N-cyclopropyl)-desosaminyl-11,12- |
| | dideoxy-3-O-decladinosyl-6-O-methyl-3-oxo-12,11-[oxycarbonyl-((4-(pyrrolo- |
| | [2,3-b]pyridin-1-yl)-butyl)-imino)]erythromycin A |
| 11. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(pyrrolo-[2,3- |
| | b]pyridin-1-yl)-butyl)-imino)]erythromycin A |
| 12. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(benzoimidazol-1- |
| | yl)-butyl)-imino)]erythromycin A |
| 13. | 2-α-Fluoro-5-O-(3′-N-desmethyl-3′-N-cyclopropyl)-desosaminyl-11,12- |
| | dideoxy-3-O-decladinosyl-6-O-methyl-3-oxo-12,11-[oxycarbonyl-((4- |
| | (benzoimidazol-1-yl)-butyl)-imino)]erythromycin A |
| 14. | 2-α-Fluoro-5-O-(3′-N-desmethyl-3′-N-n-propyl)-desosaminyl-11,12-dideoxy-3- |
| | O-decladinosyl-6-O-methyl-3-oxo-12,11-[oxycarbonyl-((4-(imidazol-1-yl)- |
| | butyl)-imino)]erythromycin A |
| 15. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(pyrrolo-[2,3-b]pyridin- |
| | 1-yl)-butyl)-imino)]erythromycin A |
| 16. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(1H-imidazo[4,5-b]- |
| | pyridin-1-yl)-butyl)-imino)]erythromycin A |
| 17. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(purin-9-yl)-butyl)- |
| | imino)]erythromycin A |
| 18. | 2-α-Fluoro-5-O-(3′-N-desmethyl-3′-N-cyclopropyl)-desosaminyl-11,12- |
| | dideoxy-3-O-decladinosyl-6-O-methyl-3-oxo-12,11-[oxycarbonyl-((4-(3H- |
| | imidazo[4,5-b]-pyridin-3-yl)-butyl)-imino)]erythromycin A |
| 19. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyridin-3-yl)- |
| | imidazol-1-yl)-butyl)-imino)]erythromycin A |
| 20. | 2α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(((N′-methyl-N′-quinolin-4- |
| | ylmethyl)-2-aminoethyl)-imino)]erythromycin A |
| 21. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(((N′-methyl-N′-pyridine-3- |
| | ylmethyl)-2-aminoethyl)-imino)]erythromycin A |
| 22. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(((N′-methyl-N-pyridine-4- |
| | ylmethyl)-2-aminoethyl)-imino)]erythromycin A |
| 23. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(((N′-methyl-N′-pyridine-2- |
| | ylmethyl)-2-aminoethyl)-imino)]erythromycin A |
| 24. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-(((N′-methyl-N′-pyridine-2- |
| | ylmethyl)-2-aminoethyl)-imino)]erythromycin A |
| 25. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-(((N′-methyl-N′-pyridine-3- |
| | ylmethyl)-2-aminoethyl)-imino)]erythromycin A |
| 26. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-(((N′-methyl-N′-pyridine-4- |
| | ylmethyl)-2-aminoethyl)-imino)]erythromycin A |
| 27. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-(((N′-methyl-N′-quinoline- |
| | 4-ylmethyl)-2-aminoethyl)-imino)]erythromycin A |
| 28. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(((N′-pyridine-3-ylmethyl)- |
| | 2-aminoethyl)-imino)]erythromycin A |
| 29. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-(((N′-acetyl-N′-pyridine-3- |
| | ylmethyl)-2-aminoethyl)-imino)]erythromycin A |
| 30. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-(((N′-pyridine-3-ylmethyl)- |
| | 2-aminoethyl)-imino)]erythromycin A |
| 31. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(((N′-acetyl-N′-pyridine-3- |
| | ylmethyl)-2-aminoethyl)-imino)]erythromycin A |
| 32. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(((N′,N′-di-pyridine-3-yl- |
| | methyl)-2-aminoethyl)-imino)]erythromycin A |
| 33. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-(((N′,N′-di-pyridine-3-yl- |
| | methyl)-2-aminoethyl)-imino)]erythromycin A |
| 34. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(quinolin-3-yl)-butyl)- |
| | imino)]erythromycin A |
| 35. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(quinolin-8-yl)-butyl)- |
| | imino)]erythromycin A |
| 36. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(pyridin-2-yl)-butyl)- |
| | imino)]erythromycin A |
| 37. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(pyridin-3-yl)-butyl)- |
| | imino)]erythromycin A |
| 38. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(pyridin-4-yl)-butyl)- |
| | imino)]erythromycin A |
| 39. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(pyridin-2-yl)-butyl)- |
| | imino)]erythromycin A |
| 40. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(pyridin-4-yl)-butyl)- |
| | imino)]erythromycin A |
| 41. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(pyridin-3-yl)-pentyl)- |
| | imino)]erythromycin A |
| 42. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(pyridin-3-yl)-butyl)- |
| | imino)]erythromycin A |
| 43. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(indol-1-yl)-butyl)- |
| | imino)]erythromycin A |
| 44. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-phenylimidazol-1- |
| | yl)-butyl)-imino)]erythromycin A |
| 45. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5-nitroindol-1-yl)- |
| | butyl)-imino)]erythromycin A |
| 46. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(1H-imidazo[4,5- |
| | c]pyridin-1-yl)-butyl)-imino)]erythromycin A |
| 47. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(benzotriazol-2-yl)- |
| | butyl)-imino)]erythromycin A |
| 48. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(3H-imidazo[4,5- |
| | c]pyridin-3-yl)-butyl)-imino)]erythromycin A |
| 49. | 2-α-Fluoro-5-O-(3′-N-desmethyl-3′-N-cyclopropyl)-desosaminyl-11,12- |
| | dideoxy-3-O-decladinosyl-6-O-methyl-3-oxo-12,11-[oxycarbonyl-((4-(4- |
| | phenylimidazol-1-yl)-butyl)-imino)]erythromycin A |
| 50. | 2-α-Fluoro-5-O-(3′-N-desmethyl-3′-N-cyclopropyl)-desosaminyl-11,12- |
| | dideoxy-3-O-decladinosyl-6-O-methyl-3-oxo-12,11-[oxycarbonyl-((4- |
| | (benzotriazol-1-yl)-butyl)-imino)]erythromycin A |
| 51. | 2-α-Fluoro-5-O-(3′-N-desmethyl-3′-N-cyclopropyl)-desosaminyl-11,12- |
| | dideoxy-3-O-decladinosyl-6-O-methyl-3-oxo-12,11-[oxycarbonyl-((4- |
| | (benzotriazol-2-yl)-butyl)-imino)]erythromycin A |
| 52. | 2-α-Fluoro-5-O-(3′-N-desmethyl-3′-N-cyclopropyl)-desosaminyl-11,12- |
| | dideoxy-3-O-decladinosyl-6-O-methyl-3-oxo-12,11-[oxycarbonyl-((4-(1H)- |
| | imdazo[4,5-b]pyridin-1-yl)-butyl)-imino)]erythromycin A |
| 53. | 2-α-Fluoro-5-O-(3′-N-desmethyl-3′-N-cyclopropyl)-desosaminyl-11,12- |
| | dideoxy-3-O-decladinosyl-6-O-methyl-3-oxo-12,11-[oxycarbonyl-((4-(indol-1- |
| | yl)-butyl)-imino)]erythromycin A |
| 54. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(benzotriazol-1-yl)- |
| | butyl)-imino)]erythromycin A |
| 55. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(benzoimidazol-1-yl)- |
| | butyl)-imino)]erythromycin A |
| 56. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(3H-imidazo[4,5- |
| | b]pyridin-3-yl)-butyl)-imino)]erythromycin A |
| 57. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(4-phenylimidazol- |
| | 1-yl)-butyl)-imino)]erythromycin A |
| 58. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(benzotriazol-2-yl)- |
| | butyl)-imino)]erythromycin A |
| 59. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(indol-1-yl)-butyl)- |
| | imino)]erythromycin A |
| 60. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(3H-imidazo[4,5- |
| | b]pyridin-3-yl)-butyl)-imino)]erythromycin A |
| 61. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(5-nitroindol-1-yl)- |
| | butyl)-imino)]erythromycin A |
| 62. | 2-α-Fluoro-5-O-(3′-N-desmethyl-3′-N-cyclopropyl)-desosaminyl-11,12- |
| | dideoxy-3-O-decladinosyl-6-O-methyl-3-oxo-12,11-[oxycarbonyl-((4-(5- |
| | nitroindol-1-yl)-butyl)-imino)]erythromycin A |
| 63. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5-fluoroindol-1-yl)- |
| | butyl)-imino)]erythromycin A |
| 64. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(benzotriazol-1-yl)- |
| | butyl)-imino)]erythromycin A |
| 65. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(2- |
| | methylbenzoimidazol-1-yl)-butyl)-imino)]erythromycin A |
| 66. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(2-ethylbenzoimidazol- |
| | 1-yl)-butyl)-imino)]erythromycin A |
| 67. | 2-α-Fluoro-5-O-(3′-N-desmethyl-3′-N-isopropyl)-desosaminyl-11,12-dideoxy-3- |
| | O-decladinosyl-6-O-methyl-3-oxo-12,11-[oxycarbonyl-((4-(benzotriazol-1-yl)- |
| | butyl)-imino)]erythromycin A |
| 68. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(2-ethylbenzoimidazol- |
| | 1-yl)-butyl)-imino)]erythromycin A |
| 69. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-((4-(pyridin-3-yl)- |
| | imidazol-1-yl)-butyl)-imino)]erythromycin A |
| 70. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(2- |
| | methylbenzoimidazol-1-yl)-butyl)-imino)] erythromycin A |
| 71. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(pyrrolo[2,3-b]pyridin- |
| | 1-yl)-butyl)-imino)]erythromycin A |
| 72. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(1H-imidazo[4,5- |
| | b]pyridin-1-yl)-butyl)-imino)]erythromycin A |
| 73. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(3H- |
| | imidazo[4,5-b]pyridin-3-yl)-butyl)-imino)]erythromycin A |
| 74. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(5- |
| | nitroindol-1-yl)-butyl)-imino)]erythromycin A |
| 75. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(2-(pyridin-3- |
| | ylmethyl)-benzoimidazol-1-yl)-butyl)-imino)]erythromycin A |
| 76. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(2-(pyridin-3-yl- |
| | methyl)-benzoimidazol-1-yl)-butyl)-imino)]erythromycin A |
| 77. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(2-phenylimidazol-1- |
| | yl)-butyl)-imino)]erythromycin A |
| 78. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(5-fluoroindol-1-yl)- |
| | butyl)-imino)]erythromycin A |
| 79. | 2-α-Fluoro-5-O-(3′-N-desmethyl-3′-N-allyl)-11,12-dideoxy-3-O-decladinosyl-6- |
| | O-methyl-3-oxo-12,11-[oxycarbonyl-((4-(5,6-dimethyl-benzoimidazol-1-yl)- |
| | butyl)-imino)]erythromycin A |
| 80. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5,6- |
| | dimethylbenzoimidazol-1-yl)-butyl)-imino)]erythromycin A |
| 81. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(3H-imidazo[4,5- |
| | c]pyridin-3-yl)-butyl)-imino)]erythromycin A |
| 82. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(2-methyl-3H- |
| | imidazo[4,5-b]pyridin-3-yl)-butyl)-imino)]erythromycin A |
| 83. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(2-methyl-3H- |
| | imidazo[4,5-b]pyridin-3-yl)-butyl)-imino)]erythromycin A |
| 84. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(1H-imidazo[4,5- |
| | c]pyridin-1-yl)-butyl)-imino)]erythromycin A |
| 85. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(6-amino-9H-purin-9- |
| | yl)-butyl)-imino)]erythromycin A |
| 86. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(2-phenylimidazol-1- |
| | yl)-butyl)-imino)]erythromycin A |
| 87. | 2-α-Fluoro-5-O-(3′-N-desmethyl-3′-N-acetyl)-desosaminyl-11,12-dideoxy-3-O- |
| | decladinosyl-6-O-methyl-3-oxo-12,11-[oxycarbonyl-((4-(benzoimidazol-1-yl)- |
| | butyl)-imino)]erythromycin A |
| 88. | 2-α-Fluoro-5-O-(3′-N-desmethyl-3′-N-cyclopropyl methyl)-desosaminyl-11,12- |
| | dideoxy-3-O-decladinosyl-6-O-methyl-3-oxo-12,11-[oxycarbonyl-((4-(3H- |
| | imidazo[4,5-b]pyridin-3-yl)-butyl)-imino)]erythromycin A |
| 89. | 2-α-Fluoro-5-O-(3′-N-desmethyl-3′-N-cyclopropyl methyl)-desosaminyl-11,12- |
| | dideoxy-3-O-decladinosyl-6-O-methyl-3-oxo-12,11-[oxycarbonyl-((4- |
| | (benzoimidazol-1-yl)-butyl)-imino)]erythromycin A |
| 90. | 2-α-Fluoro-5-O-(3′-N-desmethyl-3′-N-cyclopropyl methyl)-desosaminyl-11,12- |
| | dideoxy-3-O-decladinosyl-6-O-methyl-3-oxo-12,11-[oxycarbonyl-((4- |
| | (pyrrolo[2,3-b]pyridin-1-yl)-butyl)-imino)]erythromycin A, |
| 91. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4- |
| | (pyrrolo[2,3-b]pyridin-1-yl)-butyl)-imino)]erythromycin A |
| 92. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4- |
| | (benzotriazol-1-yl)-butyl)-imino)]erythromycin A |
| 93. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4- |
| | (benzoimidazol-1-yl)-butyl)-imino)]erythromycin A |
| 94. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(4- |
| | phenyl-imidazol-1-yl)-butyl)-imino)]erythromycin A |
| 95. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(2-trifluoromethyl- |
| | benzoimidazol-1-yl)-butyl)-imino)]erythromycin A |
| 96. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4,5-diphenyl-imidazol- |
| | 1-yl)-butyl)-imino)]erythromycin A |
| 97. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(4,5-diphenyl-imidazol- |
| | 1-yl)-butyl)-imino)]erythromycin A |
| 98. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(4-phenyl-imidazol-1- |
| | yl)-butyl)-imino)]erythromycin A |
| 99. | 2-α-Fluoro-5-O-(3′-N-desmethyl-3′-N-cyclopropyl methyl)-desosaminyl-11,12- |
| | dideoxy-3-O-decladinosyl-6-O-methyl-3-oxo-12,11-[oxycarbonyl-((4-(4- |
| | phenyl-imidazol-1-yl)-butyl)-imino)]erythromycin A |
| 100. | 2-α-Fluoro-5-O-(3′-N-desmethyl-3′-N-cyclopropyl methyl)-desosaminyl-11,12- |
| | dideoxy-3-O-decladinosyl-6-O-methyl-3-oxo-12,11-[oxycarbonyl-((4- |
| | (benzotriazol-1-yl)-butyl)-imino)]erythromycin A |
| 101. | 2-α-Fluoro-5-O-(3′-N-desmethyl-3′-N-cyclopropyl methyl)-desosaminyl-11,12- |
| | dideoxy-3-O-decladinosyl-6-O-methyl-3-oxo-12,11-[oxycarbonyl-((4-(4- |
| | (pyridin-3-yl)-imidazol-1-yl)-butyl)-imino)]erythromycin A |
| 102. | 2-α-Fluoro-5-O-(3′-N-desmethyl-3′-N-acetyl)-desosaminyl-11,12-dideoxy-3-O- |
| | decladinosyl-6-O-methyl-3-oxo-12,11-[oxycarbonyl-((4-(pyrrolo[2,3-b]pyridin- |
| | 1-yl)-butyl)-imino)]erythromycin A |
| 103. | 2-α-Fluoro-5-O-(3′-N-desmethyl-3′-N-acetyl)-desosaminyl-11,12-dideoxy-3-O- |
| | decladinosyl-6-O-methyl-3-oxo-12,11-[oxycarbonyl-((4-(4-phenyl-imidazol-1- |
| | yl)-butyl)-imino)]erythromycin A |
| 104. | 2-α-Fluoro-5-O-(3′-N-desmethyl-3′-N-acetyl)-desosaminyl-11,12-dideoxy-3-O- |
| | decladinosyl-6-O-methyl-3-oxo-12,11-[oxycarbonyl-((4-(1H-imidazo[4,5- |
| | b]pyridin-1-yl)-butyl)-imino)]erythromycin A |
| 105. | 2-α-Fluoro-5-O-(3′-N-desmethyl-3′-N-acetyl)-desosaminyl-11,12-dideoxy-3-O- |
| | decladinosyl-6-O-methyl-3-oxo-12,11-[oxycarbonyl-((4-(3H-imidazo[4,5- |
| | b]pyridin-3-yl)-butyl)-imino)]erythromycin A |
| 106. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(3,5-diphenyl)-pyrazol- |
| | 1-yl)-butyl)-imino)]erythromycin A |
| 107. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(4- |
| | (pyridn-3-yl)-imidazol-1-yl)-butyl)-imino)]erythromycin A |
| 108. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(1H- |
| | imidazo[4,5-b]pyridin-1-yl)-butyl)-imino)]erythromycin A |
| 109. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(3,5-diphenyl)-pyrazol- |
| | 1-yl)-butyl)-imino)]erythromycin A |
| 110. | 2-α-Fluoro-5-O-(3′-N-desmethyl-3′-N-cyclopropyl methyl)-desosaminyl-11,12- |
| | dideoxy-3-O-decladinosyl-6-O-methyl-3-oxo-12,11-[oxycarbonyl-((4-(1H- |
| | imidazo[4,5-b] pyridin-1-yl)-butyl)-imino)] erythromycin A |
| 111. | 2-α-Fluoro-5-O-(3′-N-desmethyl-3′-N-acetyl)-desosaminyl-11,12-dideoxy-3-O- |
| | decladinosyl-6-O-methyl-3-oxo-12,11-[oxycarbonyl-((4-(4-(pyridin-3-yl)- |
| | imidazo-1-yl)-butyl)-imino)] erythromycin A |
| 112. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(4-(2,4- |
| | difluoro-phenyl)-imidazol-1-yl)-butyl)-imino)] erythromycin A |
| 113. | 2-α-Fluoro-5-O-(3′-N-desmethyl-3′-N-2-ethyl)-desosaminyl-11,12-dideoxy-3- |
| | O-decladinosyl-6-O-methyl-3-oxo-12,11-[oxycarbonyl-((4-(4-(2,4- |
| | difluorophenyl)-imidazol-1-yl)-butyl)-imino)] erythromycin A |
| 114. | 2-α-Fluoro-5-O-(3′-N-desmethyl-3′-N-2-allyl)-desosaminyl-11,12-dideoxy-3-O- |
| | decladinosyl-6-O-methyl-3-oxo-12,11-[oxycarbonyl-((4-(4-(2,4-difluoro- |
| | phenyl)-imidazol-1-yl)-butyl)-imino)] erythromycin A |
| 115. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(furan-3-yl)- |
| | imidazol-1-yl)-butyl)-imino)] erythromycin A |
| 116. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(furan-3-yl)- |
| | imidazol-1-yl)-butyl)-imino)] erythromycin A hydrochloride salt |
| 117. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-((4-phenyl-2H-pyrazol- |
| | 1-yl)-butyl)-imino)] erythromycin A |
| 118. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyridin-3-yl)-1H- |
| | imidazol-1-yl)-pentyl)-imino)] erythromycin A |
| 119. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyridin-4-yl)-1H- |
| | imidazol-1-yl)-butyl)-imino)] erythromycin A |
| 120. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(4-(pyridin-4-yl)-1H- |
| | imidazol-1-yl)-butyl)-imino)] erythromycin A |
| 121. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-phenyl-2H-pyrazol- |
| | 1-yl)-butyl)-imino) erythromycin A |
| 122. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(1H-indazol-2-yl)- |
| | butyl)-imino)] erythromycin A |
| 123. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(1H-indazol-2-yl)- |
| | butyl)-imino)] erythromycin A |
| 124. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyridin-3-yl)-2H- |
| | pyrazol-1-yl)-butyl)-imino)] erythromycin A |
| 125. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(1H-indazol-1-yl)- |
| | butyl)-imino)] erythromycin A |
| 126. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyrazin-2-yl)-1H- |
| | imidazol-1-yl)-butyl)-imino)] erythromycin A |
| 127. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(thiophen-3-yl)-1H- |
| | imidazol-1-yl)-butyl)-imino)] erythromycin A |
| 128. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(4-(thiophen-3-yl)-1H- |
| | imidazol-1-yl)-butyl)-imino)] erythromycin A |
| 129. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(quinolin-3-yl)-1H- |
| | imidazol-1-yl)-butyl)-imino)] erythromycin A |
| 130. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(1H-indazol-1-yl)- |
| | butyl)-imino)] erythromycin A |
| 131. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyrimin-5-yl)-1H- |
| | imidazol-1-yl)-butyl)-imino)] erythromycin A |
| 132. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(4-(pyrimin-5-yl)-1H- |
| | imidazol-1-yl)-butyl)-imino)]erythromycin A |
| 133. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(furan-2-yl)-1H- |
| | imidazol-1-yl)-butyl)-imino)] erythromycin A |
| 134. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(thiophen-2-yl)-1H- |
| | imidazol-1-yl)-butyl)-imino)] erythromycin A |
| 135. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(4-chloro-phenyl)- |
| | 1H-imidazol-1-yl)-butyl)-imino)] erythromycin A |
| 136. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(4-(4-chloro-phenyl)- |
| | 1H-imidazol-1-yl)-butyl)-imino)] erythromycin A |
| 137. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyridin-2-yl)-1H- |
| | imidazol-1-yl)-butyl)-imino)] erythromycin A |
| 138. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((4-(4-(pyridin-2-yl)-1H- |
| | imidazol-1-yl)-butyl)-imino)] erythromycin A |
| 139. | 2-α-Fluoro-5-O-(3′-N-desmethyl-3′-N-allyl)-desosaminyl-11,12-dideoxy-3-O- |
| | decladinosyl-6-O-methyl-3-oxo-12,11-[oxycarbonyl-((9-(4-amino-butyl)-9H- |
| | purin-6-yl)-imino)] erythromycin A |
| 140. | 2-α-Fluoro-5-O-(3′-N-desmethyl-3′-N-2-allyl)-desosaminyl-11,12-dideoxy-3- |
| | O-decladinosyl-6-O-methyl-3-oxo-12,11-[oxycarbonyl-((4-(4-(pyridin-3-yl)- |
| | 1H-pyrazol-1-yl)-butyl)-imino)] erythromycin A |
| 141. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(4-(4- |
| | chloro-phenyl)-1H-imidazol-1-yl)-butyl)-imino)] erythromycin A |
| 142. | 2-α-Fluoro-5-O-(3′-N-desmethyl-3′-N-2-fluoroethyl)-desosaminyl-11,12- |
| | dideoxy-3-O-decladinosyl-6-O-methyl-3-oxo-12,11-[oxycarbonyl-((4-(4- |
| | (pyridin-2-yl)-1H-imidazol-1-yl)-butyl)-imino)] erythromycin A |
| 143. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(3H-imidazo[4,5- |
| | b]pyridine-2-yl)-butyl)-imino)] erythromycin A |
| 144. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(tetrazol-1-yl)- |
| | imidazol-1-yl)-butyl)-imino)] erythromycin A |
| 145. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(1H-benzoimidazol-2- |
| | yl)-butyl)-imino)] erythromycin A |
| 146. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(6-fluoro-pyridin-3- |
| | yl)-imidazol-1-yl)-butyl)-imino)] erythromycin A |
| 147. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(3H-imidazo[4,5- |
| | c]pyridine-2-yl)-butyl)-imino)] erythromycin A |
| 148. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-([1,2,4]trizol-1-yl)- |
| | phenyl)-butyl)-imino)] erythromycin A |
| 149. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(imidazol-1-yl)- |
| | phenyl)-butyl)-imino)] erythromycin A |
| 150. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(6-chloro-pyridin-3- |
| | yl)-imidazol-1-yl)-butyl)-imino)] erythromycin A |
| 151. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(3-(1-(4-aminobutyl)-1H- |
| | imidazol-4-yl)-phenyl)imino] erythromycin A |
| 152. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyridin-3-yl)- |
| | thiazol-2-yl)-butyl)-imino)] erythromycin A |
| 153. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(3-(2-(4-aminobutyl)- |
| | thiazol-4-yl)-phenyl)imino] erythromycin A |
| 154. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyrazol-1-yl)- |
| | imidazol-1-yl)-butyl)-imino)] erythromycin A |
| 155. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-([1,4′]-bipyrazolyl-1′- |
| | yl)-butyl)-imino)] erythromycin A |
| 156. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(imidazol-1-yl)- |
| | pyrazol-1-yl)-butyl)-imino)] erythromycin A |
| 157. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyrazol-1-yl)- |
| | imidazol-1-yl)-butyl)-imino)] erythromycin A |
| 158. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-([3,3′]bithiophenyl-5- |
| | yl)-butyl)-imino)] erythromycin A |
| 159. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-([2,3′]bithiophenyl-5′- |
| | yl)-butyl)-imino)] erythromycin A |
| 160. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(furan-2-yl)- |
| | thiophen-2-yl)-butyl)-imino)] erythromycin A |
| 161. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(oxazol-5-yl)- |
| | imidazol-1-yl)-butyl)-imino)] erythromycin A |
| 162. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(3-(pyrrol-1-yl)- |
| | [1,2,4]triazol-1-yl)-butyl)-imino)] erythromycin A |
| 163. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5-(thiophen-2-yl)- |
| | tetrazol-2-yl)-butyl)-imino)] erythromycin A |
| 164. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(thiophen-3-yl)- |
| | pyrazol-1-yl)-butyl)-imino)] erythromycin A |
| 165. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(furan-3-yl)-pyrazol- |
| | 1-yl)-butyl)-imino)] erythromycin A |
| 166. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(furan-2-yl)-pyrazol- |
| | 1-yl)-butyl)-imino)] erythromycin A |
| 167. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5-phenyl-tetrazol-2- |
| | yl)-butyl)-imino)] erythromycin A |
| 168. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5-(4-methoxy-phenyl)- |
| | tetrazol-2-yl)-butyl)-imino)] erythromycin A |
| 169. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5-(furan-3-yl)- |
| | imidazol-1-yl)-butyl)-imino)] erythromycin A |
| 170. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(6-pyrazol-1-yl- |
| | pyridin-3-yl)-butyl)-imino)] erythromycin A |
| 171. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(pyridin-3-yl)- |
| | pyrazol-1-yl)-butyl)-imino)] erythromycin A |
| 172. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(5-thiophen-2-yl- |
| | pyridin-3-yl)-butyl)-imino)] erythromycin A |
| 173. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-phenyl-thiophen-2- |
| | yl)-butyl)-imino)] erythromycin A |
| 174. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(N-(4-amino-butyl)-N- |
| | thiazol-2-yl-nicotinimido)]erythromycin A |
| 175. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-(N-(4-amino-butyl)-N- |
| | thiazol-2-yl-nicotinimido)]erythromycin A |
| 176. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(6-pyrrol-1-yl- |
| | pyridin-3-yl)-imidazol-1-yl)-butyl)-imino)] erythromycin A |
| 177. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(6-pyrrol-1-yl-pyridin- |
| | 3-yl)-butyl)-imino)] erythromycin A |
| 178. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(2-pyrrol-1-yl-thiazol- |
| | 5-yl)-butyl)-imino)] erythromycin A |
| 179. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(6-imidazol-1-yl- |
| | pyridin-3-yl)-butyl)-imino)] erythromycin A |
| 180. | 2-α-Fluoro-5-O-(3′-N-desmethyl-3′-N-ethyl)-desosaminyl-11,12-dideoxy-3-O- |
| | decladinosyl-6-O-methyl-3-oxo-12,11-[oxycarbonyl-((4-(4-(tetrahydro-furan-2- |
| | yl)-pyridin-3-yl]-butyl)imino] erythromycin A |
| 181. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(4-tolyl-imidazol-1-yl)-butyl)- |
| | imino)] erythromycin A |
| 182. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(4-aminophenyl)-imidazol-1-yl)- |
| | butyl)-imino)] erythromycin A |
| 183. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(4-methyl-3-aminophenyl)- |
| | imidazol-1-yl)-butyl)-imino)] erythromycin A |
| 184. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(3-(imidazol-1yl)- |
| | phenyl)imidazol-1-yl)-butyl)-imino)] erythromycin A |
| 185. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(3-(N,N-dicyclopropyl-amino)- |
| | phenyl)-imidazol-1-yl)-butyl)-imino)] erythromycin A |
| 186. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(3-(N,N-dimethyl-amino)phenyl)- |
| | imidazol-1-yl)-butyl)-imino)] erythromycin A |
| 187. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(3-(tetrazol-1yl)-phenyl)- |
| | imidazol-1-yl)-butyl)-imino)] erythromycin A |
| 188. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(2-(pyrrol-1yl)-thiozol-5-yl)- |
| | imidazol-1-yl)-butyl)-imino)] erythromycin A |
| 189. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(3-trifluoromethyl-phenyl)- |
| | imidazol-1-yl)-butyl)-imino)] erythromycin A |
| 190. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(N-(thiazol-2-yl)-benzimido)-butyl)- |
| | imino)] erythromycin A |
| 191. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(2-amino-pyrimidin-5-yl)- |
| | imidazol-1-yl)-butyl)-imino)] erythromycin A |
| 192. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(4-(pyrrol-1yl)-phenyl)-imidazol- |
| | 1-yl)-butyl)-imino)] erythromycin A |
| 193. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(3-(pyrrol-1yl)-phenyl)-imidazol- |
| | 1-yl)-butyl)-imino)] erythromycin A |
| 194. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(3-(N-acetyl-amino)-phenyl)- |
| | imidazol-1-yl)-butyl)-imino)] erythromycin A |
| 195. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(3-(N-isopropyl-amino)-phenyl)- |
| | imidazol-1-yl)-butyl)-imino)] erythromycin A |
| 196. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(3-nitrophenyl)-imidazol-1-yl)- |
| | butyl)-imino)] erythromycin A |
| 197. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(3-(N-benzoyl-amino)-phenyl)- |
| | imidazol-1-yl)-butyl)-imino)] erythromycin A |
| 198. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(3,4-dimethyl-phenyl)-imidazol- |
| | 1-yl)-butyl)-imino)] erythromycin A |
| 199. | 5-O-(3′-N-desmethyl-3′-N-ethyl)-11,12-dideoxy-3-O-decladinosyl-6-O-methyl- |
| | 3-oxo-12,11-[oxycarbonyl-((4-(4-Pyridin-3-yl-thiazol-2-yl)-butyl)-imino)] |
| | erythromycin A |
| 200. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(4-fluorophenyl)-imidazol-1-yl)- |
| | butyl)-imino)] erythromycin A |
| 201. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(4-methoxy-phenyl)-imidazol-1- |
| | yl)-butyl)-imino)] erythromycin A |
| 202. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(N-(benzthiazol-2-yl)-benzimido)- |
| | butyl)-imino)] erythromycin A |
| 203. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(N-(thiazol-2-yl)-nicotinamido)- |
| | butyl)-imino)] erythromycin A |
| 204. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((4-(4-(3-(N-methyl-amino)-phenyl)- |
| | imidazol-1-yl)-butyl)-imino)] erythromycin A |
| 205. | 2-α-Fluoro-5-O-(3′-N-desmethyl-3′-N-ethyl)desosaminyl-11,12-dideoxy-3-O- |
| | decladinosyl-6-O-methyl-3-oxo-12,11-[oxycarbonyl-((4-(4-(2-aminopyridin-4- |
| | yl)-imidazol-1-yl)-butyl)-imino)] erythromycin A |
| 206. | 2-α-Fluoro-5-O-(3′-N-desmethyl-3′-N-ethyl)desosaminyl-11,12-dideoxy-3-O- |
| | decladinosyl-6-O-methyl-3-oxo-12,11-[oxycarbonyl-((4-(4-(2-aminopyridin-5- |
| | yl)-2H-tetrazol-5-yl)-butyl)-imino)] erythromycin A |
| 207. | 2-α-Fluoro-5-O-(3′-N-desmethyl-3′-N-ethyl)desosaminyl-11,12-dideoxy-3-O- |
| | decladinosyl-6-O-methyl-3-oxo-12,11-[oxycarbonyl-((4-(4-(3,4- |
| | difluorophenyl)-imidazol-1-yl)-butyl)-imino)] erythromycin A |
| 208. | 2-α-Fluoro-5-O-(3′-N-desmethyl-3′-N-ethyl)desosaminyl-11,12-dideoxy-3-O- |
| | decladinosyl-6-O-methyl-3-oxo-12,11-[oxycarbonyl-((4-(4-(2-chloropyridin-4- |
| | yl)-imidazol-1-yl)-butyl)-imino)] erythromycin A |
| 209. | 2-α-Fluoro-5-O-(3′-N-desmethyl-3′-N-ethyl)desosaminyl-11,12-dideoxy-3-O- |
| | decladinosyl-6-O-methyl-3-oxo-12,11-[oxycarbonyl-((4-(4--(6-aminopyridin-3- |
| | yl)-imidazol-1-yl)-butyl)-imino)] erythromycin A |
| 210. | 2-α-Fluoro-5-O-(3′-N-desmethyl-3′-N-ethyl)desosaminyl-11,12-dideoxy-3-O- |
| | decladinosyl-6-O-methyl-3-oxo-12,11-[oxycarbonyl-((4-(5-(2-Chloro-pyridin-4- |
| | yl)-tetrazol-2-yl])-butyl)-imino)] erythromycin A, |
| 211. | 11,12-Dideoxy-3-O-decladinosyl-3-O-(2-nitrophenyl acetyl)-5-O-(3′-N- |
| | desmethyl-3′-N-ethyl)-6-O-methyl-12,11-[oxycarbonyl-((3-imidazol-1-yl)- |
| | propyl)-imino]erythromycin A |
| 212. | 11,12-Dideoxy-3-O-decladinosyl-3-O-(2-nitrophenyl acetyl)-5-O-(3′-N- |
| | desmethyl-3′-N-ethyl)-6-O-methyl-12,11-[oxycarbonyl-((4-imidazol-1-yl)- |
| | butyl)-imino]erythromycin A |
| 213. | 11,12-Dideoxy-3-O-decladinosyl-3-O-(2-nitrophenyl acetyl)-5-O-(3′-N- |
| | desmethyl-3′-N-allyl)-6-O-methyl-12,11-[oxycarbonyl-((3-imidazol-1-yl)- |
| | propyl)-imino]erythromycin A |
| 214. | 11,12-Dideoxy-3-O-decladinosyl-3-O-(2-nitrophenyl acetyl)-5-O-(3′-N- |
| | desmethyl-3′-N-ethyl)-6-O-methyl-12,11-[oxycarbonyl-((4-(3H)-imidazo(4,5- |
| | b)-pyridin-3-yl)-butyl)-imino]erythromycin A |
| 215. | 11,12-Dideoxy-3-O-decladinosyl-3-O-(2-nitrophenyl acetyl)-5-O-(3′-N- |
| | desmethyl-3′-N-ethyl)-6-O-methyl-12,11-[oxycarbonyl-((4-benzimidazol-1-yl)- |
| | butyl)-imino]erythromycin A |
| 216. | 11,12-Dideoxy-3-O-decladinosyl-3-O-(3-nitrophenyl acetyl)-5-O-(3′-N- |
| | desmethyl-3′-N-allyl)-6-O-methyl-12,11-[oxycarbonyl-((4-benzimidazol-1-yl)- |
| | butyl)-imino]erythromycin A |
| 217. | 11,12-Dideoxy-3-O-decladinosyl-3-O-(2-nitrophenyl acetyl)-5-O-(3′-N- |
| | desmethyl-3′-N-allyl)-6-O-methyl-12,11-[oxycarbonyl-(prop-2-en-yl)- |
| | imino]erythromycin A |
| 218. | 11,12-Dideoxy-3-O-decladinosyl-3-O-(4-pyridyl acetyl)-5-O-(3′-N-desmethyl- |
| | 3′-N-ethyl)-6-O-methyl-12,11-[oxycarbonyl-((4-imidazol-1-yl)-butyl)- |
| | imino]erythromycin A |
| 219. | 11,12-Dideoxy-3-O-decladinosyl-3-O-(2-pyridyl acetyl)-5-O-(3′-N-desmethyl- |
| | 3′-N-ethyl)-6-O-methyl-12,11-[oxycarbonyl-((4-imidazol-1-yl)-butyl)- |
| | imino]erythromycin A |
| 220. | 11,12-Dideoxy-3-O-decladinosyl-3-O-(2-pyridyl acetyl)-5-O-(3′-N-desmethyl- |
| | 3′-N-ethyl)-6-O-methyl-12,11-[oxycarbonyl-((4-benzimidazol-1-yl)-butyl)- |
| | imino]erythromycin A |
| 221. | 11,12-Dideoxy-3-O-decladinosyl-3-O-(4-pyridyl acetyl)-5-O-(3′-N-desmethyl- |
| | 3′-N-ethyl)-6-O-methyl-12,11-[oxycarbonyl-((4-benzimidazol-1-yl)-butyl)- |
| | imino]erythromycin A |
| 222. | 11,12-Dideoxy-3-O-decladinosyl-3-O-(2-pyridyl acetyl)-5-O-(3′-N-desmethyl- |
| | 3′-N-ethyl)-6-O-methyl-12,11-[oxycarbonyl-((4-(3H)-imidazol-[4,5-b]pyridin-3- |
| | yl)-butyl)-imino]erythromycin A |
| 223. | 11,12-Dideoxy-3-O-decladinosyl-3-O-(2-pyridyl acetyl)-5-O-(3′-N-desmethyl- |
| | 3′-N-ethyl)-6-O-methyl-12,11-[oxycarbonyl-((4-(1H)-imidazol-[4,5-b]pyridin-1- |
| | yl)-butyl)-imino]erythromycin A |
| 224. | 11,12-Dideoxy-3-O-decladinosyl-3-O-(2-pyridyl acetyl)-5-O-(3′-N-desmethyl- |
| | 3′-N-ethyl)-6-O-methyl-12,11-[oxycarbonyl-((4-pyrrolo-[2,3-b]pyridin-1-yl)- |
| | butyl)-imino]erythromycin A |
| 225. | 11,12-Dideoxy-3-O-decladinosyl-3-O-(2-pyridyl acetyl)-5-O-(3′-N-desmethyl- |
| | 3′-N-ethyl)-6-O-methyl-12,11-[oxycarbonyl-((4-indol-1-yl)-butyl)- |
| | imino]erythromycin A |
| 226. | 11,12-Dideoxy-3-O-decladinosyl-3-O-(2-pyridyl acetyl)-5-O-(3′-N-desmethyl- |
| | 3′-N-ethyl)-6-O-methyl-12,11-[oxycarbonyl-((4-(4-phenyl)-imidazol-1-yl)- |
| | butyl)-imino]erythromycin A |
| 227. | 11,12-Dideoxy-3-O-decladinosyl-3-O-(3-pyridyl acetyl)-5-O-(3′-N-desmethyl- |
| | 3′-N-ethyl)-6-O-methyl-12,11-[oxycarbonyl-((4-(1H)-imidazol-[4,5-b]pyridin-1- |
| | yl)-butyl)-imino]erythromycin A |
| 228. | 11,12-Dideoxy-3-O-decladinosyl-3-O-(3-pyridyl acetyl)-5-O-(3′-N-desmethyl- |
| | 3′-N-ethyl)-6-O-methyl-12,11-[oxycarbonyl-((4-indol-1-yl)-butyl)- |
| | imino]erythromycin A |
| 229. | 11,12-Dideoxy-3-O-decladinosyl-3-O-(3-pyridyl acetyl)-5-O-(3′-N-desmethyl- |
| | 3′-N-ethyl)-6-O-methyl-12,11-[oxycarbonyl-((4-benzimidazol-1-yl)-butyl)- |
| | imino]erythromycin A |
| 230. | 11,12-Dideoxy-3-O-decladinosyl-3-O-(3-pyridyl acetyl)-5-O-(3′-N-desmethyl- |
| | 3′-N-ethyl)-6-O-methyl-12,11-[oxycarbonyl-((4-pyrrolo-[2,3-b]pyridin-1-yl)- |
| | butyl)-imino]erythromycin A |
| 231. | 11,12-Dideoxy-3-O-decladinosyl-3-O-(3-pyridyl acetyl)-5-O-(3′-N-desmethyl- |
| | 3′-N-ethyl)-6-O-methyl-12,11-[oxycarbonyl-((4-(3H)-imidazol-[4,5-b]pyridin-3- |
| | yl)-butyl)-imino]erythromycin A |
| 232. | 11,12-Dideoxy-3-O-decladinosyl-3-O-(3-pyridylacetyl)-5-O-(3′-N-desmethyl-3′- |
| | N-ethyl)-6-O-methyl-12,11-[oxycarbonyl-((4-(4-phenyl)-imidazol-1-yl)-butyl)- |
| | imino]erythromycin A |
| 233. | 11,12-dideoxy-3-O-decladinosyl-3-O-(2-pyridylacetyl)-5-O-(3′-N-desmethyl-3′- |
| | N-ethyl)-6-O-methyl-12,11-[oxycarbonyl-((N¹-methyl-N¹-pyridin-4-ylmethyl)- |
| | 2-aminoethyl)-imino]erythromycin A |
| 234. | 11,12-dideoxy-3-O-decladinosyl-3-O-(2-pyridylacetyl)-5-O-(3′-N-desmethyl-3′- |
| | N-ethyl)-6-O-methyl-12,11-[oxycarbonyl-((N¹-methyl-N¹-pyridin-2-ylmethyl)- |
| | 2-aminoethyl)-imino]erythromycin A |
| 235. | 11,12-dideoxy-3-O-decladinosyl-3-O-(2-pyridylacetyl)-5-O-(3′-N-desmethyl-3′- |
| | N-ethyl)-6-O-methyl-12,11-[oxycarbonyl-((N¹-methyl-N¹-pyridin-3-ylmethyl)- |
| | 2-aminoethyl)-imino]erythromycin A |
| 236. | 11,12-dideoxy-3-O-decladinosyl-3-O-(2-pyridylacetyl)-5-O-(3′-N-desmethyl-3′- |
| | N-ethyl)-6-O-methyl-12,11-[oxycarbonyl-((N¹-methyl-N¹-quinolin-4-ylmethyl)- |
| | 2-aminoethyl)-imino]erythromycin A |
| 237. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-pyrrolo[2,3-b]pyridin-1-yl)-butyl)- |
| | imino]erythromycin A |
| 238. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-(1H)imidazo[4,5-b]pyridin-1-yl)- |
| | butyl)-imino]erythromycin A |
| 239. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-(3H)imidazo[4,5-b]pyridin-3-yl)- |
| | butyl)-imino]erythromycin A |
| 240. | 11,12-dideoxy-3-O-decladinosyl-3-O-(3-pyridylacetyl)-5-O-(3′-N-desmethyl-3′- |
| | N-ethyl)-6-O-methyl-12,11-[oxycarbonyl-((4-(2-methyl)-benzoimidazo-1-yl)- |
| | butyl)-imino]erythromycin A |
| 241. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-(4-phenyl)-imidazol-1-yl)-butyl)- |
| | imino]erythromycin A |
| 242. | 11,12-dideoxy-3-O-decladinosyl-3-O-(3-pyridylacetyl)-5-O-(3′-N-desmethyl-3′- |
| | N-ethyl)-6-O-methyl-12,11-[oxycarbonyl-((4-pyridin-2-yl)-butyl)- |
| | imino]erythromycin A |
| 243. | 11,12-dideoxy-3-O-decladinosyl-3-O-(3-pyridylacetyl)-5-O-(3′-N-desmethyl-3′- |
| | N-ethyl)-6-O-methyl-12,11-[oxycarbonyl-((4-pyridin-4-yl)-butyl)- |
| | imino]erythromycin A |
| 244. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-benzoimidazo-1-yl)-butyl)- |
| | imino]erythromycin A |
| 245. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-(4-pyridin-3-yl)-imidazol-1-yl)- |
| | butyl)-imino]erythromycin A |
| 246. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-benzotriazol-1-yl)-butyl)- |
| | imino]erythromycin A |
| 247. | 11,12-dideoxy-3-O-decladinosyl-3-O-(4-pyridylacetyl)-5-O-(3′-N-desmethyl-3′- |
| | N-ethyl)-6-O-methyl-12,11-[oxycarbonyl-((4-(4-pyridin-3-yl)-imidazol-1-yl)- |
| | butyl)-imino]erythromycin A |
| 248. | 11,12-dideoxy-3-O-decladinosyl-3-O-(3-pyridylacetyl)-5-O-(3′-N-desmethyl-3′- |
| | N-ethyl)-6-O-methyl-12,11-[oxycarbonyl-((4-(4-pyridin-3-yl)-imidazol-1-yl)- |
| | butyl)-imino]erythromycin A |
| 249. | 11,12-dideoxy-3-O-decladinosyl-3-O-(2-pyridylacetyl)-5-O-(3′-N-desmethyl-3′- |
| | N-ethyl)-6-O-methyl-12,11-[oxycarbonyl-((4-(4-pyridin-3-yl)-imidazol-1-yl)- |
| | butyl)-imino]erythromycin A |
| 250. | 11,12-dideoxy-3-O-decladinosyl-3-O-(4-pyridylacetyl)-5-O-(3′-N-desmethyl-3′- |
| | N-ethyl)-6-O-methyl-12,11-[oxycarbonyl-((4-pyrrolo[2,3-b]pyridin-1-yl)-butyl)- |
| | imino]erythromycin A |
| 251. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-(3H)imidazo[4,5-b]pyridin-3-yl)- |
| | butyl)-imino]erythromycin A |
| 252. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((9-(4-amino-butyl)9H-purin-6-yl)- |
| | imino]erythromycin A |
| 253. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-benzoimidazo-1-yl)-butyl)- |
| | imino]erythromycin A |
| 254. | 11,12-dideoxy-3-O-decladinosyl-3-O-(4-pyridylacetyl)-5-O-(3′-N-desmethyl-3′- |
| | N-ethyl)-6-O-methyl-12,11-[oxycarbonyl-((4-(4-phenyl)-imidazol-1-yl)-butyl)- |
| | imino]erythromycin A |
| 255. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-(4-phenyl)-imidazol-1-yl)-butyl)- |
| | imino]erythromycin A |
| 256. | 11,12-dideoxy-3-O-decladinosyl-3-O-(4-pyridylacetyl)-5-O-(3′-N-desmethyl-3′- |
| | N-ethyl)-6-O-methyl-12,11-[oxycarbonyl-((4-(3H)imidazo[4,5-b]pyridin-3-yl)- |
| | butyl)-imino]erythromycin A |
| 257. | 11,12-dideoxy-3-O-decladinosyl-3-O-(4-pyridylacetyl)-5-O-(3′-N-desmethyl-3′- |
| | N-ethyl)-6-O-methyl-12,11-[oxycarbonyl-((4-benzotriazol-1-yl)-butyl)- |
| | imino]erythromycin A |
| 258. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-(4-pyridin-3-yl)-imidazol-1-yl)- |
| | butyl)-imino]erythromycin A |
| 259. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-pyrrolo[2,3-b]pyridin-1-yl)-butyl)- |
| | imino]erythromycin A |
| 260. | 11,12-dideoxy-3-O-decladinosyl-3-O-(2-pyridylacetyl)-5-O-(3′-N-desmethyl-3′- |
| | N-propargyl)-6-O-methyl-12,11-[oxycarbonyl-((4-benzoimidazo-1-yl)-butyl)- |
| | imino]erythromycin A |
| 261. | 11,12-dideoxy-3-O-decladinosyl-3-O-(2-pyridylacetyl)-5-O-(3′-N-desmethyl-3′- |
| | N-ethyl)-6-O-methyl-12,11-[oxycarbonyl-((4-benzotriazol-1-yl)-butyl)- |
| | imino]erythromycin A |
| 262. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-(1H)imidazo[4,5-b]pyridin-1-yl)- |
| | butyl)-imino]erythromycin A |
| 263. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-benzotriazol-1-yl)-butyl)- |
| | imino]erythromycin A |
| 264. | 11,12-dideoxy-3-O-decladinosyl-3-O-(3-pyridylacetyl)-5-O-(3′-N-desmethyl-3′- |
| | N-ethyl)-6-O-methyl-12,11-[oxycarbonyl-((4-benzotriazol-1-yl)-butyl)- |
| | imino]erythromycin A |
| 265. | 11,12-dideoxy-3-O-decladinosyl-3-O-(2-pyridylacetyl)-5-O-(3′-N-desmethyl-3′- |
| | N-propargyl)-6-O-methyl-12,11-[oxycarbonyl-((4-pyrrolo[2,3-b]pyridin-1-yl)- |
| | butyl)-imino]erythromycin A |
| 266. | 11,12-dideoxy-3-O-decladinosyl-3-O-(2-pyridylacetyl)-5-O-(3′-N-desmethyl-3′- |
| | N-allyl)-6-O-methyl-12,11-[oxycarbonyl-((4-(4-furan-2-yl)-imidazol-1-yl)- |
| | butyl)-imino]erythromycin A |
| 267. | 11,12-dideoxy-3-O-decladinosyl-3-O-(3-pyridylacetyl)-5-O-(3′-N-desmethyl-3′- |
| | N-allyl)-6-O-methyl-12,11-[oxycarbonyl-((4-(4-thiophen-2-yl)-imidazol-1-yl)- |
| | butyl)-imino]erythromycin A |
| 268. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(3-(3[4-(6-amino-9H-purin-9- |
| | yl)propyl)hydrazo)] erythromycin A |
| 269. | 11,12-dideoxy-3-O-decladinosyl-3-O-(2-pyridyl acetyl)-5-O-(3′-N-desmethyl- |
| | 3′-N-ethyl)-6-O-methyl-12,11-[oxycarbonyl-((4-(quinolin-8-yl)-butyl)- |
| | imino)]erythromycin A |
| 270. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(quinolin-8-yl)-butyl)- |
| | imino)]erythromycin A |
| 271. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(quinolin-4-yl)-butyl)- |
| | imino)]erythromycin A |
| 272. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(1-methyl-1H-pyrrolo[2,3- |
| | b]pyridin-3-yl)-butyl)-imino)]erythromycin A |
| 273. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(6-amino-9H-purin-9-yl |
| | butylimino)] erythromycin A |
| 274. | 11,12-dideoxy-3-O-decladinosyl-3-O-(3-pyridyl acetyl)-5-O-(3′-N-desmethyl- |
| | 3′-N-ethyl)-6-O-methyl-12,11-[oxycarbonyl-(4-(6-amino-9H-purin-9-yl |
| | butylimino)] erythromycin A |
| 275. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(isoquinolin-5-yl)-butyl)- |
| | imino)]erythromycin A |
| 276. | 11,12-dideoxy-3-O-decladinosyl-3-O-(2-pyridyl acetyl)-5-O-(3′-N-desmethyl- |
| | 3′-N-ethyl)-6-O-methyl-12,11-[oxycarbonyl-((4-(isoquinolin-5-yl)-butyl)- |
| | imino)]erythromycin A |
| 277. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(4-thiophen-3-yl-1H-imidazol- |
| | 1-yl)-butyl)-imino)]erythromycin A |
| 278. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(4-thiophen-3-yl-1H-imidazol- |
| | 1-yl)-butyl)-imino)]erythromycin A hydrochloride salt |
| 279. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(6-dimethylamino-9H-purin-9-yl |
| | butylimino)]erythromycin A |
| 280. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(6-pyrrol-1-yl-purin-9-yl)- |
| | butyl)-imino)] erythromycin A |
| 281. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(6-diethylamino-9H-purin-9-yl |
| | butylimino)] erythromycin A |
| 282. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(6-ethylamino-9H-purin-9-yl |
| | butylimino)] erythromycin A |
| 283. | 11,12-dideoxy-3-O-decladinosyl-3-O-(2-pyridyl acetyl)-5-O-(3′-N-desmethyl- |
| | 3′-N-ethyl)-6-O-methyl-12,11-[oxycarbonyl-(4-(6-dimethylamino-9H-purin-9- |
| | yl butylimino)] erythromycin A |
| 284. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(6-dimethylamino-9H-purin-9-yl |
| | butylimino)]erythromycin A |
| 285. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(6-methylamino-9H-purin-9-yl |
| | butylimino)]erythromycin A |
| 286. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(6-methylamino-9H-purin-9-yl |
| | butylimino)]erythromycin A |
| 287. | 11,12-dideoxy-3-O-decladinosyl-3-O-(2-pyridyl acetyl)-5-O-(3′-N-desmethyl- |
| | 3′-N-2-fluoroethyl)-6-O-methyl-12,11-[oxycarbonyl-((4-(3H-imidazo[4,5- |
| | b]pyridin-3-yl)-butyl)-imino)]erythromycin A |
| 288. | 11,12-dideoxy-3-O-decladinosyl-3-O-(2-pyridyl acetyl)-5-O-(3′-N-desmethyl- |
| | 3′-N-2-fluoroethyl)-6-O-methyl-12,11-[oxycarbonyl-((4-(1H-imidazo[4,5- |
| | b]pyridin-1-yl)-butyl)-imino)]erythromycin A |
| 289. | 11,12-dideoxy-3-O-decladinosyl-3-O-(2-pyridyl acetyl)-5-O-(3′-N-desmethyl- |
| | 3′-N-2-fluoroethyl)-6-O-methyl-12,11-[oxycarbonyl-(4-(6-amino-9H-purin-9-yl |
| | butylimino)] erythromycin A |
| 290. | 11,12-dideoxy-3-O-decladinosyl-3-O-(3-pyridyl acetyl)-5-O-(3′-N-desmethyl- |
| | 3′-N-2-fluoroethyl)-6-O-methyl-12,11-[oxycarbonyl-(4-(6-amino-9H-purin-9-yl |
| | butylimino)] erythromycin A |
| 291. | 11,12-dideoxy-3-O-decladinosyl-3-O-(3-pyridyl acetyl)-5-O-(3′-N-desmethyl- |
| | 3′-N-2-fluoroethyl)-6-O-methyl-12,11-[oxycarbonyl-((4-(1H-imidazo[4,5- |
| | b]pyridin-1-yl)-butyl)-imino)] erythromycin A |
| 292. | 11,12-dideoxy-3-O-decladinosyl-3-O-(3-pyridyl acetyl)-5-O-(3′-N-desmethyl- |
| | 3′-N-2-fluoroethyl)-6-O-methyl-12,11-[oxycarbonyl-((4-(3H-imidazo[4,5- |
| | b]pyridin-3-yl)-butyl)-imino)] erythromycin A |
| 293. | 11,12-dideoxy-3-O-decladinosyl-3-O-(3-pyridyl acetyl)-5-O-(3′-N-desmethyl- |
| | 3′-N-ethyl)-6-O-methyl-12,11-[oxycarbonyl-(4-(6-dimethylamino-9H-purin-9- |
| | yl butylimino)] erythromycin A |
| 294. | 11,12-dideoxy-3-O-decladinosyl-3-O-(3-pyridyl acetyl)-5-O-(3′-N-desmethyl- |
| | 3′-N-ethyl)-6-O-methyl-12,11-[oxycarbonyl-(4-(6-methylamino-9H-purin-9-yl |
| | butylimino)] erythromycin A |
| 295. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-((4-(purin-9-yl)-butyl)- |
| | imino)]erythromycin A |
| 296. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(purin-9-yl)-butyl)- |
| | imino)]erythromycin A |
| 297. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(1H-imidazo[4,5-c]pyridin-1- |
| | yl)-butyl)-imino)]erythromycin A |
| 298. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-((4-(3H-imidazo[4,5-c]pyridin-3- |
| | yl)-butyl)-imino)]erythromycin A |
| 299. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(4-thiophen-2-yl-1H-imidazol- |
| | 1yl)-butyl)-imino)]erythromycin A |
| 300. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-((4-(1H-imidazol[4,5-c]pyridin-1- |
| | yl)-butyl)-imino)]erythromycin A |
| 301. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(3H-imidazol[4,5-c]pyridin-3- |
| | yl)-butyl)-imino)]erythromycin A |
| 302. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-((4-(isoquinolin-5-yl)-butyl)- |
| | imino)]erythromycin A |
| 303. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(4-oxazol-5-yl-1H-imidazol- |
| | 1yl)-butyl)-butyl)-imino)]erythromycin A |
| 304. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-((4-(4-furan-2-yl-1H-imidazol-1yl)- |
| | butyl)-imino)] erythromycin A |
| 305. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-((4-(4-furan-2-yl-1H-imidazol-1yl)- |
| | butyl)-imino)] erythromycin A hydrochloride salt |
| 306. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-((4-(4-furan-3-yl-1H-imidazol-1yl)- |
| | butyl)-imino)]erythromycin A |
| 307. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(4-furan-3-yl-1H-imidazol-1yl)- |
| | butyl)-imino)]erythromycin A |
| 308. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(thiophen-2-yl)-pyrazol-1-yl)- |
| | butylimino)] erythromycin A |
| 309. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-(furan-2-yl)-pyrazol-1-yl)- |
| | butylimino)] erythromycin A |
| 310. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(furan-2-yl)-pyrazol-1-yl)- |
| | butylimino)] erythromycin A |
| 311. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-(thiophen-2-yl)-1H-pyrazol- |
| | 1-yl)-butylimino)] erythromycin A |
| 312. | 11,12-dideoxy-3-O-decladinosyl-3-O-(2-pyridyl acetyl)-5-O-(3′-N-desmethyl- |
| | 3′-N-ethyl)-6-O-methyl-12,11-[oxycarbonyl-(4-(6-amino-9H-purin-9-yl |
| | butylimino)]erythromycin A |
| 313. | 11,12-dideoxy-3-O-decladinosyl-3-O-(3-pyridyl acetyl)-5-O-(3′-N-desmethyl- |
| | 3′-N-ethyl)-6-O-methyl-12,11-[oxycarbonyl-((4-(quinolin-3-yl)-butyl)- |
| | imino)]erythromycin A |
| 314. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(quinolin-3-yl)-butyl)- |
| | imino)]erythromycin A |
| 315. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(2-thiazolyl)-imidazol-1-yl)- |
| | butylimino)] erythromycin A |
| 316. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-(thiazol-2-yl)-imidazol-1-yl)- |
| | butylimino)] erythromycin A |
| 317. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(furan-3-yl)-pyrazol-1-yl)- |
| | butylimino)] erythromycin A |
| 318. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(thiophen-3-yl)-pyrazol-1-yl)- |
| | butylimino)] erythromycin A |
| 319. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-(thiophen-3-yl)-pyrazol-1-yl)- |
| | butylimino)] erythromycin A |
| 320. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-(furan-3-yl)-pyrazol-1-yl)- |
| | butylimino)] erythromycin A |
| 321. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((3-(4-pyridin-3-yl-1H-imidazol-1- |
| | yl)-propyl)-hydrazo)]erythromycin A |
| 322. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(3-benzoimidazol-1-yl)-propyl)- |
| | hydrazo)] erythromycin A |
| 323. | (R or S) 11,12-dideoxy-3-O-decladinosyl-3-O-(2-pyridyl acetyl)-5-O-(3′-N- |
| | desmethyl-3′-N-allyl)-6-O-methyl-12,11-[oxycarbonyl-((3-(4-pyridin-3-yl-1H- |
| | imidazol-1yl)-propyl)-hydrazo)]erythromycin A |
| 324. | (S or R) 11,12-dideoxy-3-O-decladinosyl-3-O-(2-pyridyl acetyl)-5-O-(3′-N- |
| | desmethyl-3′-N-allyl)-6-O-methyl-12,11-[oxycarbonyl-((3-(4-pyridin-3-yl-1H- |
| | imidazol-1-yl)-propyl)-hydrazo)]erythromycin A |
| 325. | 11,12-dideoxy-3-O-decladinosyl-3-O-(3-pyridyl acetyl)-5-O-(3′-N-desmethyl- |
| | 3′-N-ethyl)-6-O-methyl-12,11-[oxycarbonyl-(3-(4-pyridin-3-yl-1H-imidazol-1- |
| | yl)-propyl)-hydrazo)]erythromycin A |
| 326. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(3-(4-pyridin-3-yl-1H-imidazol- |
| | 1yl)-propyl)-hydrazo)]erythromycin A |
| 327. | 11,12-dideoxy-3-O-decladinosyl-3-O-(2-pyridyl acetyl)-5-O-(3′-N-desmethyl- |
| | 3′-N-ethyl)-6-O-methyl-12,11-[oxycarbonyl-(3-(4-pyridin-3-yl-1H-imidazol- |
| | 1yl)-propyl)-hydrazo)]erythromycin A |
| 328. | 11,12-dideoxy-3-O-decladinosyl-3-O-(2-pyridyl acetyl)-5-O-(3′-N-desmethyl- |
| | 3′-N-ethyl)-6-O-methyl-12,11-[oxycarbonyl-(3-(3-(6-amino-9H-purin-9-yl- |
| | propyl)hydrazo)]erythromycin A |
| 329. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(3-(4-(thiophen-3-yl)-1H-imidazol- |
| | 1yl)-propyl)-hydrazo)]erythromycin A |
| 330. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(3-isoquinolin-5-yl)-propyl)- |
| | hydrazo)]erythromycin A |
| 331. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(3-purin-9-yl)-propyl)- |
| | hydrazo)]erythromycin A |
| 332. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((3-purin-9-yl)-propyl)- |
| | hydrazo)]erythromycin A |
| 333. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(3-(4-(furan-2-yl)-1H-imidazol-1- |
| | yl)propyl)-hydrazo)]erythromycin A |
| 334. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(3-(3-(6-amino-9H-purin-9-yl- |
| | propyl)hydrazo)] erythromycin A |
| 335. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(3-(4-(furan-3-yl)-1H-imidazol-1- |
| | yl)propyl)-hydrazo)]erythromycin A |
| 336. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(3-(4-(furan-3-yl)-1H-imidazol-1- |
| | yl)propyl)-hydrazo)]erythromycin A |
| 337. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(3-(3-(6-amino-9H-purin-9-yl- |
| | propyl)hydrazo)]erythromycin A |
| 338. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(3-(4-(thiophen-2-yl-1H-imidazol- |
| | 1-yl)-propyl)-hydrazo)]erythromycin A |
| 339. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(3-(4-(thiophen-2-yl-1H-imidazol- |
| | 1-yl)-propyl)-hydrazo)]erythromycin A |
| 340. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(3-(4-(thiophen-3-yl-1H-imidazol- |
| | 1-yl)-propyl)-hydrazo)]erythromycin A |
| 341. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(3-(imidazo[4,5-b]pyridin-1-yl)- |
| | propyl)-hydrazo)] erythromycin A |
| 342. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(3-(imidazo[4,5-b]pyridin-3-yl)- |
| | propyl)-hydrazo)] erythromycin A |
| 343. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(3-(4-(thiazol-2-yl-1H-imidazol-1- |
| | yl)-propyl)-hydrazo)]erythromycin A |
| 344. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(3-(4-(thiazol-2-yl-1H-imidazol-1- |
| | yl)-propyl)-hydrazo)]erythromycin A |
| 345. | (R or S)11,12-dideoxy-3-O-decladinosyl-3-O-(2-pyridyl acetyl)-5-O-(3′-N- |
| | desmethyl-3′-N-allyl)-6-O-methyl-12,11-[oxycarbonyl-hydrazo]erythromycin A |
| 346. | (S or R) 11,12-dideoxy-3-O-decladinosyl-3-O-(2-pyridyl acetyl)-5-O-(3′-N- |
| | desmethyl-3′-N-allyl)-6-O-methyl-12,11-[oxycarbonyl-hydrazo]erythromycin A |
| 347. | 11,12-dideoxy-3-O-decladinosyl-3-O-(3-pyridyl acetyl)-5-O-(3′-N-desmethyl- |
| | 3′-N-ethyl)-6-O-methyl-12,11-[oxycarbonyl-hydrazo)]erythromycin A |
| 348. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((3-(4-pyridin-3-yl-1H-imidazol-1- |
| | yl)-propyl)-hydrazo)]erythromycin A, 9-(O-methyl)oxime |
| 349. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-hydrazo]erythromycin A, 9-(O- |
| | methyl)oxime |
| 350. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-((4-(4-(6-fluoro-pyridin-3-yl)-1H- |
| | imidazol-1-yl)-butyl)-imino)]erythromycin A |
| 351. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(4-(6-fluoro-pyridin-3-yl)-1H- |
| | imidazol-1-yl)-butyl)-imino)]erythromycin A |
| 352. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(4-(1H-imidazol-1-yl)phenyl)- |
| | butyl)-imino)]erythromycin A |
| 353. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-((4-(5-(3-aminophenyl)-thiazol-2- |
| | yl)-butyl)-imino)]erythromycin A |
| 354. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(2-amino-pyrimidin)-5-yl)- |
| | imidazol-1-yl)-butylimino)] erythromycin A |
| 355. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(2-aminopyridin-4-yl)- |
| | imidazol-1-yl)-butylimino)] erythromycin A |
| 356. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(6-aminopyridin-3-yl)- |
| | imidazol-1-yl)-butylimino)] erythromycin A |
| 357. | 11,12-dideoxy-3-O-decladinosyl-3-O-(3-pyridyl acetyl)-5-O-(3′-N-desmethyl- |
| | 3′-N-ethyl)-6-O-methyl-12,11-[oxycarbonyl-(4-(6-(dimethylamino)-9H-purin-9- |
| | yl)-butylimino)] erythromycin A |
| 358. | 11,12-dideoxy-3-O-decladinosyl-3-O-(2-nitrophenyl acetyl)-5-O-(3′-N- |
| | desmethyl-3′-N-allyl)-6-O-methyl-12,11-[oxycarbonyl-(3-(5-phenyl-tetrazol-1- |
| | yl)propylimino)] erythromycin A |
| 359. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-pyridin-3-yl-pyrazol-1-yl)- |
| | butylimino)] erythromycin A |
| 360. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(2-methyl-2H-tetrazol-5- |
| | yl)phenyl)butylimino)] erythromycin A |
| 361. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-(3-thienyl)-imidazol-1- |
| | yl)butylimino)] erythromycin A |
| 362. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(5-methyl-[1,3,4]-oxadiazol- |
| | 2-yl)phenyl)butylimino)] erythromycin A |
| 363. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-([2,3′]-bithiophenyl-5- |
| | yl)butylimino)] erythromycin A |
| 364. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-([2,3′]-bithiophenyl5- |
| | yl)butylimino)] erythromycin A |
| 365. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-[3,3′]-bithiophenyl-5- |
| | yl)butylimino)] erythromycin A |
| 366. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-[3,3′]-bithiophenyl-5- |
| | yl)butylimino)] erythromycin A |
| 367. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(5-pyridin-3-yl)-tetrazol-2- |
| | yl)butylimino)] erythromycin A |
| 368. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(5-(furan-2-yl)-imidazol-1- |
| | yl)butylimino)] erythromycin A |
| 369. | 11,12-dideoxy-3-O-decladinosyl-3-O-(2-methylpropanoyl)-5-O-(3′-N- |
| | desmethyl-3′-N-allyl)-6-O-methyl-12,11-[oxycarbonyl4-(4-(4-(furan-2-yl)- |
| | imidazol-1-yl)-butylimino)] erythromycin A |
| 370. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(4-methoxy-phenyl)- |
| | imidazol-1-yl)-butylimino)] erythromycin A |
| 371. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-(4-methoxy-phenyl)- |
| | imidazol-1-yl)-butylimino)] erythromycin A |
| 372. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-(2-methoxy-phenyl)- |
| | imidazol-1-yl)-butylimino)] erythromycin A |
| 373. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(2-methoxy-phenyl)-1H- |
| | imidazol-1-yl)-butylimino)] erythromycin A |
| 374. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-phenyl-thiophen-2-yl)- |
| | butylimino)] erythromycin A |
| 375. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-phenyl-thiophen-2-yl)- |
| | butylimino)] erythromycin A |
| 376. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-furan-2-yl-thiophen-2- |
| | yl)butylimino)] erythromycin A |
| 377. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-furan-2-yl-thiophen-2-yl)- |
| | butylimino)] erythromycin A |
| 378. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(5-(4-methoxyphenyl)-tetrazol- |
| | 2-yl)-butylimino)] erythromycin A |
| 379. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(5-(4-methoxyphenyl)-tetrazol- |
| | 2-yl)-butylimino)] erythromycin A |
| 380. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(5-phenyl-tetrazol-2-yl)- |
| | butylimino)] erythromycin A |
| 381. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(5-pyridin-4-yl-tetrazol-2-yl)- |
| | butylimino)] erythromycin A |
| 382. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(5-pyridin-4-yl-tetrazol-2-yl)- |
| | butylimino)] erythromycin A |
| 383. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(5-phenyl-tetrazol-2-yl)- |
| | butylimino)] erythromycin A |
| 384. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(6-chloropyridin-3-yl)- |
| | imidazol-1-yl)butylimino)] erythromycin A |
| 385. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-3((3-(4-(imidazol-1-yl)-pyrazol-1- |
| | yl)-propyl)-hydrazo)] erythromycin A |
| 386. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(3-(3-(4-pyrazol-1-yl]-imidazol-1- |
| | yl)-propyl)-hydrazo)] erythromycin A |
| 387. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-(3-aminophenyl)-imidazol-1- |
| | yl)-butylimino)] erythromycin A |
| 388. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-3-((3-([1,4′]-bipyrazol-1′-yl)- |
| | propyl)-hydrazo)] erythromycin A |
| 389. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(3-aminophenyl)-imidazol-1- |
| | yl)-butylimino)] erythromycin A |
| 390. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-pyrimidin-5-yl-imidazol-1- |
| | yl)butylimino)] erythromycin A |
| 391. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(3-(3-(4-pyrimidin-2-yl-imidazol-1- |
| | yl)-propyl)-hydrazo] erythromycin A |
| 392. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-pyrimidin-5-yl-imidazol-1- |
| | yl)butylimino)] erythromycin A |
| 393. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(5-(furan-2-yl)-tetrazol-2- |
| | yl]butylimino)] erythromycin A |
| 394. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(5-(thiophen-2-yl)-tetrazol-2- |
| | yl])-butylimino)] erythromycin A |
| 395. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(5-(furan-2-yl)-tetrazol-1-yl]- |
| | butylimino)] erythromycin A |
| 396. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(5-(furan-2-yl)-tetrazol-1-yl]- |
| | butylimino)] erythromycin A |
| 397. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(5-(furan-2-yl)-tetrazol-2- |
| | yl]butylimino)] erythromycin A |
| 398. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(5-pyridin-3-yl-tetrazol-2-yl)- |
| | butylimino)] erythromycin A |
| 399. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(5-pyridin-2-yl-tetrazol-2-yl)- |
| | butylimino)] erythromycin A |
| 400. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(5-pyridin-2-yl-tetrazol-2-yl)- |
| | butylimino)] erythromycin A |
| 401. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(5-thiophen-2-yl-tetrazol-1-yl)- |
| | butylimino)] erythromycin A |
| 402. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(5-thiophen-2-yl-tetrazol-1-yl)- |
| | butylimino)] erythromycin A |
| 403. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(5-(thiophen-2-yl-tetrazol-2-yl)- |
| | butylimino)] erythromycin A |
| 404. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(1H-benzimidazol-2-yl)- |
| | butylimino)] erythromycin A |
| 405. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(1H-imidazol[4,5-b]pyridin-2- |
| | yl)-butylimino)] erythromycin A |
| 406. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(1H-imidazol[4,5-b]pyridin-2-yl)- |
| | butylimino)] erythromycin A |
| 407. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-tetrazol-1-yl)-imidazol-1-yl)- |
| | butylimino)] erythromycin A |
| 408. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-tetrazol-1-yl)-imidazol-1-yl)- |
| | butylimino)] erythromycin A |
| 409. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(1H-benzimidazol-2-yl)- |
| | butylimino)] erythromycin A |
| 410. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(3-({3-[4-(6-fluoro-pyridin-3-yl- |
| | imidazol-1-yl] propyl}hydrazo] erythromycin A |
| 411. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(3-({3-[4-(6-fluoro-pyridin-3-yl- |
| | imidazol-1-yl] propyl}hydrazo] erythromycin A |
| 412. | 11,12-dideoxy-3-O-decladinosyl-3-O-(2-methyl propanoyl)-5-O-(3′-N- |
| | desmethyl-3′-N-allyl)-6-O-methyl-12,11-[oxycarbonyl-(4-(benzimidazol-1-yl)- |
| | butylimino)] erythromycin A |
| 413. | 11,12-dideoxy-3-O-decladinosyl-3-O-(2-methyl propanoyl)-5-O-(3′-N- |
| | desmethyl-3′-N-allyl)-6-O-methyl-12,11-[oxycarbonyl-(4-(4-(3-pyridyl)- |
| | imidazol-1-yl)-butylimino)] erythromycin A |
| 414. | 11,12-dideoxy-3-O-decladinosyl-3-O-(2-methyl propanoyl)-5-O-(3′-N- |
| | desmethyl-3′-N-allyl)-6-O-methyl-12,11-[oxycarbonyl-(4-(4-imidazol[4,5- |
| | b]pyridin-1-yl)-butylimino)] erythromycin A |
| 415. | 11,12-dideoxy-3-O-decladinosyl-3-O-(2-methyl propanoyl)-5-O-(3′-N- |
| | desmethyl-3′-N-allyl)-6-O-methyl-12,11-[oxycarbonyl-(4-(6-amino-9H-purin-9- |
| | yl)butylimino)] erythromycin A |
| 416. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-([1,4′]-bipyrazol-1′- |
| | yl)butylimino] erythromycin A |
| 417. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-[4-(pyrazol-1-yl)-imidazol-1- |
| | yl)butylimino] erythromycin A |
| 418. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-([1,4′]-biimidazol-1′-yl) |
| | butylimino)] erythromycin A |
| 419. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-([1,4′]-biimidazol-1′- |
| | yl)butylimino)] erythromycin A |
| 420. | 11,12-dideoxy-3-O-decladinosyl-3-O-(2-methylpropanoyl)-5-O-(3′-N- |
| | desmethyl-3′-N-allyl)-6-O-methyl-12,11-[oxycarbonyl-(4-(imidazo[4,5- |
| | b]pyridin-3-yl)butylimino)] erythromycin A |
| 421. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-pyrazol-1-yl)-imidazol-1- |
| | yl)butylimino)] erythromycin A |
| 422. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(1H-imidazo[4,5-c]pyridin-2- |
| | yl)butylimino)] erythromycin A |
| 423. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-(1H-imidazo[4,5-c]pyridin-2- |
| | yl)butylimino)] erythromycin A |
| 424. | 11,12-dideoxy-3-O-decladinosyl-3-O-(2-methylpropanoyl)-5-O-(3′-N- |
| | desmethyl-3′-N-allyl)-6-O-methyl-12,11-[oxycarbonyl-(4-(4-(6-fluoro-pyridin- |
| | 3-yl)-imidazol-1-yl)butylimino)] erythromycin A |
| 425. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-phenyl-thiazol-2- |
| | yl)butylimino)] erythromycin A |
| 426. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-phenyl-thiazol-2- |
| | yl)butylimino)] erythromycin A |
| 427. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(3-((3-(4-pyrimidin-2-yl)-imidazol- |
| | 2-yl)propyl)-hydrazo)] erythromycin A |
| 428. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-3-((3-(4-pyrimidin-5-yl)-imidazol- |
| | 2-yl)propyl)-hydrazo)] erythromycin A |
| 429. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-[4-(1H-[1,2,4]-triazol-1- |
| | yl)phenyl]butylimino)] erythromycin A |
| 430. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-[4-(1H-[1,2,4]-triazol-1- |
| | yl)phenyl]butylimino)] erythromycin A |
| 431. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-pyrimidin-5-yl- |
| | phenyl)butylimino)] erythromycin A |
| 432. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(3-(3-(4-pyrimidin-5-yl-imidazol-1- |
| | yl)propyl)hydrazo)] erythromycin A |
| 433. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(3-({3-[4-(imidazol-1-yl)-pyrazol- |
| | 1-yl]propyl}hydrazo)] erythromycin A |
| 434. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(3-{[3-([1,4′]-biimidazol-1′- |
| | yl)propyl}hydrazo)] erythromycin A |
| 435. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(3-(3-(4-pyrazol-1-yl)-imidazol-1- |
| | yl)-propyl)-hydrazo] erythromycin A |
| 436. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-3-((3-(1H-imidazo[4,5-b]pyridine- |
| | 1-yl)-propyl)-hydrazo)] erythromycin A |
| 437. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-(oxazol-5-yl)-imidazol-1- |
| | yl)butylimino)] erythromycin A |
| 438. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-imidazol-1-yl)-pyrazol-1- |
| | yl]butylimino)] erythromycin A |
| 439. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-imidazol-1-yl)-pyrazol-1- |
| | yl]butylimino)] erythromycin A |
| 440. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-(2H-tetrazol-5-yl)- |
| | phenyl)butylimino)] erythromycin A |
| 441. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-[4-(2H-tetrazol-5-yl)- |
| | phenyl]butylimino)] erythromycin A |
| 442. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-(4-(4-(6-chloropyridin-3-yl)- |
| | imidazol-1-yl]butylimino)] erythromycin A |
| 443. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-3-((3-([1,4′]-bipyrazol-1-yl)-)- |
| | propyl)-hydrazo)] erythromycin A |
| 444. | 11,12-dideoxy-3-O-decladinosyl-3-O-(2-nitrophenyl acetyl)-5-O-(3′-N- |
| | desmethyl-3′-N-allyl)-6-O-methyl-12,11-[oxycarbonyl-(4-benzimidazol-1- |
| | yl)butylimino)] erythromycin A |
| 445. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-(4-(4-(6-fluoro-pyridin-3-yl)- |
| | imidazol-1-yl)-butylimino)] erythromycin A |
| 446. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-(4-(4-(2-amino-pyrimidin)-5-yl)- |
| | pyrazol-1-yl l)-butyl)-imino)] erythromycin A |
| 447. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-((4-(4-tetrazol-1-yl-imidazol-1-yl)- |
| | butyl)-imino)] erythromycin A |
| 448. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-(4-(4-(3-(N-acetyl-amino)-phenyl)- |
| | imidazol-1-yl)-butyl)-imino)] erythromycin A |
| 449. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-(4-(4-(4-fluoro-phenyl)-imidazol-1-yl)- |
| | butyl)-imino)] erythromycin A |
| 450. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-((4-(4-(3-(N,N-dimethyl-amino)- |
| | phenyl)-imidazol-1-yl)-butyl)-imino)] erythromycin A |
| 451. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-(4-(4-(4-methoxy-phenyl)-imidazol-1- |
| | yl)-butyl)-imino)] erythromycin A |
| 452. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-(4-(4-(2-amino-pyridin)-5-yl)-imidazol- |
| | 1-yll)-butyl)-imino)] erythromycin A |
| 453. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-(4-(4-(N-(benzthiazol-2-yl)-benzamido)- |
| | butyl)-imino)] erythromycin A |
| 454. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-((4-(4-furan-3-yl-imidazol-1-yl)-butyl)- |
| | imino)] erythromycin A |
| 455. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-((4-(4-furan-2-yl-imidazol-1-yl)-butyl)- |
| | imino)] erythromycin A |
| 456. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-(4-(4-(3-(N-isopropyl-amino)-phenyl)- |
| | imidazol-1-yl)-butyl)-imino)] erythromycin A |
| 457. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-(4-(4-(N-(thiazol-2-yl)-nicotinamido)- |
| | butyl)-imino)] erythromycin A |
| 458. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-((4-(6-imidazol-1-yl-pyridin-3-yl)- |
| | butyl)-imino)] erythromycin A |
| 459. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-(4-(4-(N-(thiazol-2-yl)-benzamido)- |
| | butyl)-imino)] erythromycin A |
| 460. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-((4-(2-pyrrol-1-yl-thiazol-5-yl)-butyl)- |
| | imino)] erythromycin A |
| 461. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-((4-(4-(3-(N,N-dicyclopropyl-amino)- |
| | phenyl)-imidazol-1-yl)-butyl)-imino)] erythromycin A |
| 462. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-((4-(4-(2-pyrrol-1-yl-thiazol-5-yl)- |
| | imidazol-1-yl)-butyl)-imino)] erythromycin A |
| 463. | 5-O-(3′-N-desmethyl-3′-N-ethyl)desosaminyl-11,12-dideoxy-3-O-decladinosyl- |
| | 6-O-methyl-3-oxo-12,11-[oxycarbonyl-((4-(4-thiaophen-2-yl-pyridin-3-yl)- |
| | butyl)-imino)] erythromycin A |
| 464. | 11,12-dideoxy-3-O-decladinosyl-6-O-methyl-3-oxo-12,11-[oxycarbonyl-(4-(4- |
| | (4-methyl-3-aminophenyl)-imidazol-1-yl]-butylimino] erythromycin A |
| 465. | 2-Methyl-11,12-dideoxy-3-O-decladinosyl-6-O-methyl-3-oxo-12,11- |
| | [oxycarbonyl-4-(4-(3-amino-4-methylphenyl)-imidazol-1-yl)-butylimino] |
| | erythromycin A |
| 466. | 11,12-dideoxy-3-O-decladinosyl-6-O-methyl-3-oxo-12,11-[oxycarbonyl-(4-(N- |
| | (2-thiazolyl)-nicotinamido)-butylimino] erythromycin A |
| 467. | 2-Methyl-11,12-dideoxy-3-O-decladinosyl-6-O-methyl-3-oxo-12,11- |
| | [oxycarbonyl-(4-(4-Imidazo[4,5-b]pyridin-3-yl)-butylimino] erythromycin A |
| 468. | 2-Methyl-11,12-dideoxy-3-O-decladinosyl-6-O-methyl-3-oxo-12,11- |
| | [oxycarbonyl-4-(4-(4-fluorophenyl)-imidazol-1-yl)-butylimino] erythromycin A |
| 469. | 2-α-Fluoro-5-O-(3′-N-desmethyl-3′-N-ethyl)-11,12-dideoxy-3-O-decladinosyl- |
| | 6-O-methyl-3-oxo-12,11-[oxycarbonyl-((3-phenyl-allyl)-hydrazo)]erythromycin A |
| 470. | 2-α-Fluoro-5-O-(3′-N-desmethyl-3′-N-allyl)-11,12-dideoxy-3-O-decladinosyl-6- |
| | O-methyl-3-oxo-12,11-[oxycarbonyl-((3-(4-thiophen-3-yl-imidazol-1- |
| | yl)propyl)-hydrazo)] erythromycin A |
| 471. | 11,12-dideoxy-3-O-decladinosyl-3-O-(3-pyridyl acetyl)-5-O-(3′-N-desmethyl- |
| | 3′-N-allyl)-6-O-methyl-12,11-[oxycarbonyl-imino)]erythromycin A |
| 472. | 11,12-dideoxy-3-O-decladinosyl-3-O-(2-pyridyl acetyl)-5-O-(3′-N-desmethyl- |
| | 3′-N-allyl)-6-O-methyl-12,11-[oxycarbonyl-imino)]erythromycin A |

3. The use according to claim 1-2, wherein the medicament further comprises one or more carriers, excipients, diluents or mixture thereof.

4. The use of macrolide derivatives for the manufacture of a medicament for treating or preventing acne vulgaris and associated inflammatory conditions as claimed in claim 1-2, wherein the medicament is a combination pharmaceutical composition comprising at least one other therapeutic agent.

5. The use according to claim 4, wherein the combination pharmaceutical composition is a single composition comprising:
a. one or more 14-membered macrolide derivatives listed in claim 1 or 2;
b. one or more therapeutic agents selected from alcohol, benzoyl peroxide, clindamycin, tretinoin, vitamin E, vitamin A and its derivatives, tetracycline, isotretinoin, vitamin C, vitamin D, chaparral, dandelion root, licoric root, Echinacea, kelp, cayenine, sassafras, elder flowers, pantothenic acid, para amino benzoic acid, biotin, cholin, inositol, folic acid, calcium, magnesium, potassium, vitamin B₆, zinc, carotenoid, azelaic acid or mixture thereof, and
c. one or more pharmaceutically acceptable carriers, excipients, diluents or mixture thereof.

6. The use according to claim 4, wherein the combination pharmaceutical composition comprises:
a. a first pharmaceutical composition comprising one or more 14-membered macrolide derivatives included in claim 1 or 2 and one or more pharmaceutically acceptable carriers, excipients, diluents or mixture thereof;
b. a second pharmaceutical composition comprising one or more therapeutic agents selected from alcohol, benzoyl peroxide, clindamycin, tretinoin, vitamin E, vitamin A and its derivatives, tetracycline, isotretinoin, vitamin C, vitamin D, chaparral, dandelion root, licoric root, Echinacea, kelp, cayenine, sassafras, elder flowers, pantothenic acid, para amino benzoic acid, biotin, cholin, inositol, folic acid, calcium, magnesium, potassium, vitamin B₆, zinc, carotenoid, azelaic acid or mixture thereof, and one or more pharmaceutically acceptable carriers, excipients, diluents or mixture thereof.

7. The use according to claim 4, wherein combination pharmaceutical compositions are administered separately, sequentially or simultaneously.
